(19) United States
(12) Patent Application Publication
(10) Pub. No.: US 2026/0335247 A1
(43) Pub. Date: Oct. 29, 2026

(54) EXOSOME-MEDIATED TARGETED DRUG DELIVERY TO TREAT CANCER

(71) Applicant: The Johns Hopkins University, Baltimore, MD (US)

(72) Inventor: Ranjan J. Perera, Orlando, FL (US)

(21) Appl. No.: 19/655,973

(22) Filed: Apr. 23, 2026

Related U.S. Application Data
(60) Provisional application No. 63/793,352, filed on Apr. 23, 2025.

(51) Int. Cl. A61K 9/50; A61K 31/165; A61K 31/167; A61K 31/404; A61K 31/4402; A61K 31/454; A61K 31/4745; A61K 31/502; A61K 31/5025; A61K 31/519; A61K 31/5377; A61K 31/55; A61K 31/7068; A61K 31/7105; A61P 35/00

(52) U.S. Cl. CPC A61K 9/5068; A61K 31/165; A61K 31/167; A61K 31/404; A61K 31/4402; A61K 31/454; A61K 31/4745; A61K 31/502; A61K 31/5025; A61K 31/519; A61K 31/5377; A61K 31/55; A61K 31/7068; A61K 31/7105; A61P 35/00

(57) ABSTRACT

The present invention relates to the field of cancer. More specifically, the present invention provides methods and compositions useful for detecting and treating prostate cancer and medulloblastoma. In one embodiment, the present invention provides a composition comprising an exosome isolated from a B7H3 chimeric antigen receptor (CAR) T-cell loaded with small interfering ribonucleic acids (siRNAs) to tetratricopeptide 3 (TTC3) and/or epithelial cell adhesion molecule (EpCAM). In another embodiments, the present invention comprises a composition comprising an exosome isolated from a B7H3 chimeric antigen receptor (CAR) T-cell loaded with small interfering ribonucleic acids (siRNAs) to glutathione peroxidase 4 (GPX4) and/or acyl-CoA synthetase long-chain family member 4 (ACSL4). In yet another embodiment, the exosome is further loaded with microRNA-211 (miR-211).

### CROSS-REFERENCE TO RELATED APPLICATIONS

This application claims the benefit of U.S. Provisional Application No. 63/793,352, filed Apr. 23, 2025, which is incorporated herein by reference in its entirety.

### FIELD OF THE INVENTION

The present invention relates to the field of cancer. More specifically, the present invention provides methods and compositions useful for detecting and treating prostate cancer and medulloblastoma.

### REFERENCE TO AN ELECTRONIC SEQUENCE LISTING

The text of the computer readable sequence listing filed herewith, titled “P18400-02”, created Apr. 22, 2026, having a file size of 81,484 bytes, is hereby incorporated by reference in its entirety.

### BACKGROUND OF THE INVENTION

Despite significant advances in therapy, improvements in prostate cancer (PCa)-related mortality have been marginal at best; mortality has even increased among some selected American population subgroups [1]. According to 2024 National Cancer Institute estimates, there will be 300,000 new PCa diagnoses, of which 35,250 are predicted to be fatal [2]. Although many men are still treated with prostate surgery [3], most men with PCa have indolent disease for which conservative therapy or an active surveillance approach would be more appropriate and result in less treatment-related morbidity [4]. However, maximizing the detection of treatable PCa that risks the individual and limiting the detection of indolent disease to minimize unnecessary procedures remain challenging. Although still widely used, prostate-specific antigen (PSA) testing has poor cancer specificity, and it does not differentiate indolent from aggressive disease, resulting in large numbers of unnecessary biopsies and overtreatment [5, 6]. While Gleason grading is important for guiding subsequent management, separating high-risk from low-risk PCa remains problematic. It is conceivable that important molecular events, regardless of the overall tumor grade, might identify individuals with a worse prognosis and serve as new targets for therapy.

### SUMMARY OF THE INVENTION

As described herein, in one aspect, the present inventor has developed a novel therapeutic approach for inducing tumor immunogenicity, activating immunosurveillance anti-tumor responses, and reducing tumor growth using B3-H7-CAR-T-Exosomes enriched with small interfering ribonucleic acids (siRNAs) to tetratricopeptide 3 (TTC3), epithelial cell adhesion molecule (EpCAM) and/or prostate cancer antigen 3 (PCA3).

The results described herein show that CAR-T cell-derived exosomes (CAR-Exo) can effectively induce anti-tumor immune responses with low immunotoxicity and deliver tumor suppressor agents to reduce PCa tumor growth. Anti-B7H3-CAR-Exos harboring si-TTC3, si-EpCAM and/or PCA3 are expected to induce efficient immunotherapeutic reactions in patients with PCa without associated systemic toxicities. The present inventor will test the hypothesis using CAR-Exos in a humanized mouse model (HuCD34-NCG) and the (C57BL/6-Tg (TRAMP)8247Ng/J×FVB/NJ) F1/J prostate cancer model. Adding siRNAs (si-TTC3, si-EpCAM and/or PCA3) and small molecules into micro-vesicles (exosomes) to target tumors is novel and will generate important therapeutic value for men with prostate cancer in the United States and worldwide.

In another aspect, the present invention provides similar compositions for the treatment of medulloblastoma (MB). In particular embodiments, exosomes derived from B3-H7 CAR-T cells are loaded with siRNA to glutathione peroxidase 4 (GPX4) and/or acyl-CoA synthetase long-chain family member 4 (ACSL4). The exosomes can further be loaded with microRNA 211 (miR-211). The exosomes can also be loaded with H2.0-like homeobox transcription factor (HLX), MYC and/or insulin-like growth factor 2 mRNA-binding protein 3 (IGF2BP3).

Accordingly, in one embodiment, a composition comprises an exosome isolated from a B7H3 chimeric antigen receptor (CAR) T-cell loaded with small interfering ribonucleic acids (siRNAs) to tetratricopeptide 3 (TTC3) and/or epithelial cell adhesion molecule (EpCAM). In some embodiments, the exosome is further loaded with siRNA to prostate cancer antigen 3 (PCA3). In other embodiments, the exosome is further loaded with a small molecule cancer therapeutic. In another embodiment, the composition further comprises a small molecular cancer therapeutic. The small molecule cancer therapeutic can comprise a polyadenosine diphosphate-ribose polymerase (PARP) inhibitor. In some embodiments, the PARP inhibitor comprises rucaparib, olaparib, talazoparib or niraparib. In particular embodiments, the present invention provides a method for treating prostate cancer in a subject comprising the step of administering a composition described herein.

The present invention also provides a composition comprising an extracellular vesicle isolated from a CAR T-cell loaded with a ribonucleic acid interference (RNAi) molecule. In specific embodiments, the CAR comprises B7H3. In particular embodiments, the extracellular vesicle comprises an exosome. In certain embodiments, the RNAi molecule comprises siRNA. In some embodiments, the siRNA targets TTC3 and/or EpCAM. In a more specific embodiment, the siRNA further targets PCA3.

The extracellular vesicle can further be loaded with a small molecule cancer therapeutic. In other embodiments, the composition further comprises a small molecule cancer therapeutic. The small molecule cancer therapeutic can comprise a PARP inhibitor including, but not limited to, rucaparib, olaparib, talazoparib and niraparib. In particular embodiments, the present invention provides a method for treating prostate cancer in a subject comprising the step of administering a composition described herein.

The present invention also provides a composition comprising an exosome isolated from a B7H3 chimeric antigen receptor (CAR) T-cell loaded with small interfering ribonucleic acids (siRNAs) to glutathione peroxidase 4 (GPX4) and/or acyl-CoA synthetase long-chain family member 4 (ACSL4). In further embodiments, the exosome is loaded with microRNA-211 (miR-211). In some embodiments, the exosome is further loaded with siRNAs to H2.0-like homeobox transcription factor (HLX), MYC, and/or insulin-like growth factor 2 mRNA-binding protein 3 (IGF2BP3). In other embodiments, the exosome is further loaded with a small molecule cancer therapeutic. Alternatively, the composition further comprises a small molecular cancer therapeutic. In particular embodiments, the present invention provides a method for treating prostate cancer in a subject comprising the step of administering a composition described herein.

In other embodiments, the siRNA targets GPX4, ACSL4, HLX, MYC and/or IGF2BP3. In some embodiments, the extracellular vesicle is further loaded with miR-211. The extracellular vesicle can further be loaded with a small molecule cancer therapeutic. Alternatively, the composition further comprises a small molecule cancer therapeutic. The composition can include, but is not limited to, pemetrexed, gemcitabine, vismodegib, sonidegib, panobinostat, romidepsin, belinostat, CI-994 (tacedinaline), silmitasertib, palbociclib, sapanisertib, or a combination of palbociclib and sapanisertib. In some embodiments, the present invention provides a method for treating medullobastoma in a subject comprising the step of administering a composition described herein.

### BRIEF DESCRIPTION OF THE FIGURES

FIG. 1A-1C. Expression of B7-H3 in PCa using sequencing data. (FIG. 1A) Expression of 7-H3 in PCa tumors by PCa DB. (FIG. 1B) Link between B7-H3 and the overall survival of PCa patients. (FIG. 1C) Expression of B7-H3 in PC3 cells.

FIG. 2A-2G. B7-H3 (mbA)-CAR-T cell-derived exosomes expressing B7-H3 (mbA) on the surface. (FIG. 2A-2B) Expression of B7-H3 (mbA) on CAR-T cells compared to T cells. (FIG. 2C) Expression of B7-H3 (mbA) on CAR-T-derived exosomes. (FIG. 2D-2F) Characterization of B7-H3(mbA) CAR-Exos including concentration, size, and protein markers. (FIG. 2G) Exo-Brite specific marker of exosomes.

FIG. 3A-3D. B7-H3 (mbA)-CAR-T-Exos loaded with si-TTC3 and/or si-EpCAM and efficiently knocked down target genes. (FIG. 3A) Expression of EpCAM in CAR-Exos/si-EpCAM/si-EpCAM+si-TTC3 treated PC3 cells. (FIG. 3B) Expression of TTC3 in CAR-Exos/si-TTC3/si-TTC3+si-EpCAM-treated PC3 cells. (FIG. 3C) Western blot results showed the same knockdown efficiency of CAR-Exos targets. (FIG. 3D) Proliferation of tumor cells is inhibited by B7-H3-CAR-Exos loaded with si-TTC3 and/or si-EpCAM.

FIG. 4. B7-H3 (mbA)-CAR-T-Exos can efficiently induce naïve T cell activation by increasing levels of IFN-7.

FIG. 5A-5C. EpCAM-CAR T exosomes efficiently delivered si-TTC3-Alexa fluor 647 to the tumor tissue. (FIG. 5A-5B) Tail vein injection of CAR-T-derived exosomes loaded with si-TTC3 labeled with Alexa fluor 647 showed promising and specific delivery of si-TTC3 to the tumor tissue. (FIG. 5C) Although some CAR-T exosomes significantly accumulated in the tumor, there was some exosome accumulation in other organs.

FIG. 6A-6C. B7-H3 (CD276) is significantly overexpressed in MB tumors, particularly in G3-MB. (FIG. 6A) RNA-Seq of 1505 MB patients (GSE124814). (FIG. 6B) qPCR validation of B7-H3 expression in MBs cell lines. (FIG. 6C) detection of B7-H3 on the surface of G3-MB cells (D425 cells). Unpublished results. ***p<0.0001, unpaired t-test.

FIG. 7A-7B. GPX4 is overexpressed in G3-MB tumor tissue and cell lines. (FIG. 7A) The expression of GPX4 in MB patients. (FIG. 7B) The expression of GPX4 in MB cell lines. ***p<0.0001, unpaired t-test. results.

FIG. 8A-8F. Generation of anti-B7-H3 (CD276)-CAR T exosomes. (FIG. 8A-8C) B7-H3 (mbA) was conjugated with GFP in transduced CAR-T cells and CAR-Exosomes harvested. (FIG. 8D-8E) Characterization of the quality (size and concentration) of isolated exosomes. (FIG. 8F) Protein marker confirmation of isolated exosomes.

FIG. 9A-9E. Anti-B7-H3 (CD276)-CAR T exosomes target D425 cells and deliver therapeutic siRNA. (FIG. 9A) B7-H3 (mbA) CAR-Exos discriminated D425 cells and penetrated efficiently. (FIG. 9B-9D) B7-H3-CAR-Exos delivered si-ACSL4 and/or si-GPX4 and significantly knocked down the expression of target genes in D425 cells. (FIG. 9E) Quantifying protein levels in all groups showed a significant knockdown of the target genes by CAR-T-derived exosomes. *p<0.05, **p<0.01, students' t-test.

FIG. 10. Anti-B7-H3 (CD276)-CAR T Exo/si-GPX4 induces ferroptosis in D425 cells. The results show that CAR-Exos enriched with si-GPX4 significantly increase ferroptosis (middle panel) of D425 cells. B7-H3-CAR-Exo+si-NC (negative control), B7-H3-CAR-Exo+si-GPX4 (si-GPX4 knockdown), RSL3 (positive control).

FIG. 11. B7-H3 (mbA)-CAR T exosomes promote T cell activation. The levels of IFN-7 were significantly increased compared to non-activated and positive controls. B7-H3(mbA)-CAR-Exos were added to the culture of naïve T cells for 72 h, and then the levels of IFN-7 were assessed by intracellular staining. Panels left to right are negative control, positive control (anti-CD3/CD28), and B7-H3(mbA)-CAR-T-Exos.

FIG. 12. CAR T exosomes efficiently pass the blood-brain barrier and accumulate in the tumor tissue. CAR-Exos labeled with Cy5.5 and injected by tail vein injection in NOD/SCID mice showed promising delivery to the brain tumor with high efficiency to pass BBB and target tumor tissue.

FIG. 13. The generation of B7-H3-CAR-T-Exos-miR-211 for targeting G3-MB

FIG. 14A-14M. T cell-Exo-miR-211 successfully delivered miR-211 to D425 cell lines and significantly downregulated the expression of ACSL4. (FIG. 14A-14B) Viability and sorting of CD8⁺ T cells. (FIG. 14C) Growth and expansion of CD8⁺ T cells in serum-free RPMI. (FIG. 14D-14G) Characterization of Exos using protein assay and NTA. (FIG. 14H) Exos delivered EGFP and TR-siRNA to D425 cells and enhanced fluorescent expression. (FIG. 14I-14M) T cell-Exo-miR-211 significantly increased levels of miR-211 in D425 cells and downregulated the expression of ACSL4.

FIG. 15. Expression levels of EPHA2 in normal tissues. The protein expression of EPHA2 in normal tissues is obviously very low.

FIG. 16A-16E. Clinical Database of MB patients.

FIG. 17. The expression of EPHA2 protein in the MB cell lines.

FIG. 18. The generation of EPHA2(mAb)-CAR-T and CTL-CAR-T cells.

FIG. 19A-19B. Efficient GPX4 knockdown by CAR-T-derived exosomes loaded with si-GPX4. Quantitative RT-PCR analysis of GPX4 expression following treatment with CAR-T-derived exosomes (B7-H3 CAR-T Exos) loaded with si-GPX4. FIG. 19A: Bar graph shows GPX4 mRNA levels normalized to ACTB and expressed as fold change relative to si-NC control. Control groups (Mock, si-NC, B7-H3 CAR-T Exos, and B7-H3 CAR-T Exos-si-NC) show no significant change in GPX4 expression. In contrast, treatment with si-GPX4 alone or B7-H3 CAR-T Exos-si-GPX4 results in robust suppression of GPX4 expression (~82-86% knockdown). The combination of B7-H3 CAR-T Exos-si-GPX4 with the ferroptosis inducer RSL3 maintains strong knockdown efficiency. FIG. 19B: table presents the corresponding Ct values for ACTB (reference gene) and GPX4 across all treatment groups, confirming consistent normalization and effective suppression of the target gene. Data are presented as mean±SEM from independent experiments. These results demonstrate that CAR-T exosome-mediated delivery of si-GPX4 achieves efficient gene silencing, supporting its potential to induce ferroptosis in tumor cells.

FIG. 20. B7-H3 CAR-T-derived exosomes loaded with si-GPX4 enhance lipid peroxidation in D425 medulloblastoma cells. Lipid peroxidation was assessed by flow cytometry using the BODIPY-C11 probe in D425 cells under the indicated treatment conditions. Representative scatter plots (top panel) show BODIPY-C11 fluorescence (Y585-PE-A) with gated populations (red boxes) indicating oxidized lipid species. Quantification reveals a progressive increase in lipid peroxidation from Mock (83.74%) and CAR-T exosomes alone (94.82%) to si-NC (89.24%) and si-GPX4 treatment (95.30%), with the highest levels observed in cells treated with B7-H3 CAR-T Exos-si-GPX4 in combination with RSL3.

Histograms (bottom panel) display fluorescence intensity shifts of BODIPY-C11, with rightward shifts (green population) indicating increased lipid oxidation. The proportion of oxidized (V1R) versus reduced (V1L) populations shows a marked increase in lipid peroxidation in the si-GPX4 and combination-treated groups compared with controls.

These results indicate that GPX4 silencing via CAR-T exosome delivery, particularly when combined with the ferroptosis inducer RSL3, significantly enhances lipid peroxidation, consistent with induction of ferroptotic cell death.

FIG. 21. Quantification of lipid peroxidation demonstrates enhanced ferroptosis following GPX4 targeting and combination therapy. Flow cytometry-based quantification of lipid peroxidation in D425 medulloblastoma cells under the indicated treatment conditions. The percentage of cells undergoing lipid peroxidation (green) versus non-lipid peroxidation (red) is shown. Control groups (Mock and si-NC) exhibit low basal lipid peroxidation, whereas GPX4 silencing (si-GPX4) and pharmacologic induction with RSL3 significantly increase lipid peroxidation.

Treatment with B7-H3(mAb) CAR-T-derived exosomes alone shows minimal induction of lipid peroxidation; however, delivery of si-GPX4 via CAR-T exosomes markedly enhances lipid peroxidation. The combination of B7-H3(mAb) CAR-T Exos-si-GPX4 with RSL3 produces the highest levels of lipid peroxidation, indicating a synergistic effect in promoting ferroptosis.

Data are presented as mean±SEM from independent experiments, with individual data points shown. Statistical significance is indicated as ***p<0.001 compared to si-NC or relevant control groups. These results support the efficacy of CAR-T exosome-mediated GPX4 targeting, particularly when combined with RSL3, in inducing ferroptotic cell death in tumor cells.

FIG. 22. CAR-T exosome-mediated GPX4 silencing reshapes the tumor immune microenvironment. Flow cytometry-based profiling of tumor-infiltrating leukocytes in tumors treated with B7-H3(mAb) CAR-T-derived exosomes carrying si-GPX4 compared with si-NC controls. Cellular composition is shown as a percentage of total cells for individual tumors (si-NC-1, si-NC-2 vs. si-GPX4-1, si-GPX4-2). Populations analyzed include total tumor-associated macrophages (TAMs; black), CD14⁺F4/80\hat{ }high TAMs (red), CD14⁺Ly6C\hat{ }high TAMs (green), CD14⁻CD49b⁺ NK cells (purple), and CD11c⁺ dendritic cells (orange).

GPX4 silencing via CAR-T exosomes results in a marked reduction in total TAMs and immunosuppressive macrophage subsets, accompanied by a significant increase in NK cell infiltration. Dendritic cell populations remain relatively unchanged.

Data are presented as mean±SEM with individual data points shown. Statistical significance is indicated as p<0.01 ( ) and p<0.001 ( ) compared with si-NC controls. These findings suggest that induction of ferroptosis through GPX4 targeting promotes a more immune-reactive tumor microenvironment.

FIG. 23. CAR-T-derived exosomes efficiently deliver si-GPX4 into D425 medulloblastoma cells. Representative fluorescence microscopy images showing uptake and intracellular localization of B7-H3(mAb) CAR-T-derived exosomes loaded with si-GPX4 in D425 cells. Nuclei are stained with Hoechst (blue), CAR-T exosomes are labeled with GFP (green), and si-GPX4 is conjugated to AF647 (red).

In cells treated with CAR-T exosomes (top row), strong GFP signal indicates efficient exosome uptake, and punctate AF647 fluorescence demonstrates successful intracellular delivery of si-GPX4. The merged images show co-localization of exosomes and siRNA within tumor cells. In contrast, control D425 cells without exosome treatment (bottom row) show minimal to no AF647 signal, confirming the specificity of siRNA delivery via CAR-T exomes.

Scale bars: 20 m. These results demonstrate effective cellular delivery of therapeutic siRNA by CAR-T exosome-based nanosystems.

FIG. 24. CAR-T-derived exosomes loaded with si-EpCAM downregulate EpCAM and TTC3 expression in PC3 prostate cancer cells. Quantitative RT-PCR analysis of EpCAM and TTC3 mRNA expression in PC3 cells following treatment with B7-H3(mAb) CAR-T-derived exosomes (CAR-T-E) loaded with siRNAs. Cells (200,000 per condition) were treated with CAR-T exosomes (125 ng/μL) with or without siRNA for 48 hours. Gene expression levels were normalized to ACTB. Left panel: EpCAM expression. Treatment with B7-H3 CAR-T exosomes carrying si-EpCAM significantly reduces EpCAM mRNA levels compared to CAR-T exosomes alone and si-NC controls. Co-treatment conditions (e.g., si-TTC3 or combination groups) show no additional significant reduction (ns), indicating specificity of si-EpCAM-mediated knockdown. Right panel: TTC3 expression. Delivery of si-EpCAM via CAR-T exosomes results in a significant decrease in TTC3 mRNA levels, suggesting downstream regulation. Combination treatments further reduce TTC3 expression compared to controls.

FIG. 25. CAR-T-derived exosomes loaded with si-HLX efficiently knock down HLX expression in D425 medulloblastoma cells. D425 cells (300,000 per condition) were treated with B7-H3(mAb) CAR-T-derived exosomes (150 g) loaded with si-HLX (2.5 μL; stock 10 mM) for 48 hours. HLX mRNA expression was quantified by qRT-PCR and normalized to ACTB. Left panel: Relative HLX expression in D425 cells. Treatment with CAR-T exosomes carrying si-HLX results in a significant reduction in HLX expression compared to Mock and si-NC controls (***p<0.001), demonstrating effective target gene silencing. Middle panel: Fold change in HLX expression relative to CAR-E-si-NC controls. Replicate samples (HLX1 and HLX2) show consistent knockdown (~70-80% reduction), while Mock and si-NC groups remain unchanged. Right panel: Corresponding Ct values for ACTB (reference gene) and HLX across all samples, confirming consistent normalization and efficient HLX suppression in si-HLX-treated groups. Data are presented as mean±SEM with individual data points shown. These results demonstrate that CAR-T exosome-mediated delivery of si-HLX enables robust and reproducible gene knockdown in medulloblastoma cells.

### DETAILED DESCRIPTION OF THE INVENTION

It is understood that the present invention is not limited to the particular methods and components, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present invention.

### I. DEFINITIONS

“Nucleic acid” is used in its broadest sense and comprises ribonucleic acids (RNA) and deoxyribonucleic acids (DNA) from all possible sources, in all lengths and configurations, such as double-stranded, single-stranded, circular, linear or branched. All sub-units and sub-types are also comprised, such as oligomers, plasmids, viral and bacterial nucleic acids, as well as genomic and non-genomic DNA and RNA from animal and plant cells or other eukaryotes or prokaryotes, messenger RNA (mRNA) in processed and unprocessed form, transfer RNA (tRNA), heterogeneous nuclear RNA (hnRNA), ribosomal RNA (rRNA), mitochondrial RNA (mtRNA), nRNA (nuclear RNA), siRNA (short interfering RNA), small nuclear RNA (snRNA), small nucleolar RNA (snoRNA), small Cajal Body specific RNA (scaRNA), micro RNA (miRNA), doubled-stranded RNA (dsRNA), ribozyme, riboswitch, viral RNA, double-stranded DNA (dsDNA), single-stranded DNA (ssDNA), plasmid DNA, cosmid DNA, chromosomal DNA, viral DNA, mitochondrial DNA (mtDNA), nuclear DNA (nDNA), small nuclear DNA (snDNA), signal recognition particle RNA (SRP RNA), antisense RNA (asRNA), Y RNA, telomerase RNA, or the like.

The terms “nucleic acid,” “polynucleotide,” “polynucleic acid,” and “oligonucleotide” and their grammatical equivalents can be used interchangeably and can refer to a deoxyribonucleotide or ribonucleotide polymer, in linear or circular conformation, and in either single- or double-stranded form. For the purposes of the present disclosure, these terms should not be construed as limiting with respect to length. The terms can also encompass analogues of natural nucleotides, as well as nucleotides that are modified in the base, sugar and/or phosphate moieties (e.g., phosphorothioate backbones). Modifications of the terms can also encompass demethylation, addition of CpG methylation, removal of bacterial methylation, and/or addition of mammalian methylation. In general, an analogue of a particular nucleotide can have the same base-pairing specificity, i.e., an analogue of A can base-pair with T.

The term “sequence” and its grammatical equivalents as used herein can refer to a nucleotide sequence, which can be DNA or RNA; can be linear, circular or branched; and can be either single-stranded or double stranded. A sequence can be mutated. A sequence can be of any length, for example, between 2 and 1,000,000 or more nucleotides in length (or any integer value there between or there above), e.g., between about 100 and about 10,000 nucleotides or between about 200 and about 500 nucleotides.

“Peptide”, “polypeptide” and “protein” are used interchangeably to refer to amino acid sequences i.e., two or more amino acids linked by a peptide bond.

“Extracellular vesicles” refer to membrane-derived microvesicles, which includes a range of vesicles, including exosomes, microparticles and shed microvesicles secreted by many cell types under both normal physiological and pathological conditions. The methods and compositions described herein can be applied to microvesicles of various sizes; for example, 30 to 200 nm, for example, 30 to 800 nm, for example, up to 2 um.

As used herein, the terms “exosome” and “microvesicle” are used interchangeably herein with the terms “microvesicle,” “liposome,” “exosome,” “exosome-like particle,” “exosome-like vesicle,” “nano-vector,” “archeosome,” “lactosome,” “extracellular vesicle,” “argosome,” “apoptotic body,” “epididimosome,” “exosome-like vesicle,” “microparticle,” “promininosome,” “prostasome,” “dexosome,” “texosome,” and “oncosome,” and grammatical variations of each of the foregoing.

“Purified” when used in reference to an exosome/microvesicle/extracellular vesicle refers to the fact that it is removed from the majority of other cellular components from which it was generated or in which it is typically present in nature.

The term “exosome” as used herein refers to any extracellular vesicle derived from any bodily fluid from a human or an animal (e.g., blood), any extracellular vesicle derived from human or animal cell lines, cell cultures, and primary cultures not limited to autologous exosomes, universal donor exosomes, allogenic exosomes, and modified exosomes. In certain examples, the exosome is made to meet pharmaceutical and cGMP standards.

The term “CAR T-cell exosome” refers to an exosome isolated/derived from a CAR T-cell.

The term “cargo” as used herein refers to any type of molecule or any type of RNA including small interfering RNA (siRNA), miRNA, mRNA, tRNA, rRNA, RNAi, regulating RNA, gRNA, long interference RNA, non-coding and coding RNA; any type of DNA including DNA fragments, DNA plasmids, interference DNA (DNAi); including any type of nucleic acid including antisense oligonucleotides (ASO); any genetic material; any genetic construct; any nucleic acid construct; any plasmid or vector; any protein including recombinant endogenous protein, enzyme, antibody, wnt signaling proteins; any lipid; any therapeutic molecule or diagnostic molecule; any cellular component; any virus including retrovirus, AdV, AAV of any variety and strain, and DNA viruses; any gene editing technology including clustered regularly interspaced short palindromic repeats (CRISPR), CRISPR/CAS9 system, any endonucleases for base editing, a Zinc finger, a single base editor, Transcription activator-like effector nucleases (TALENs), any meganuclease; any synthetic molecular conjugate; or combination thereof loaded into an exosome. Typically, such cargo is naturally not present in the exosome. In certain examples, the cargo is made to meet pharmaceutical and cGMP standards.

“siRNA” is an agent which functions to inhibit expression of a target gene by RNA interference (RNAi). siRNA forms a double stranded RNA and has the ability to reduce or inhibit expression of a gene or target gene when the siRNA is present or expressed in the same cell as the target gene.

“Target nucleic acid” or “target polynucleotide” or “target RNA” refers to a nucleic acid molecule that has a function which is desired to be inhibited. In particular embodiments, a target comprises TTC3, EpCAM and/or PCA3. In other embodiments, a target comprises GPX4, ACSL4, HLX, MYC and/or IGF2BP3.

“Transfection” refers to the introduction of nucleic acid into a cell or extracellular vesicle. Examples of methods of transfection include, but are not limited to, electroporation, calcium phosphate, lipofection, and viral infection utilizing a viral vector. An siRNA can be introduced into a cell or EV in a non-expressible form. An siRNA can be introduced into a cell or EV in an expressible form (e.g., within an expression vector).

“Patient”, “subject” and “individual” are used interchangeably herein, and refer to an animal, particularly a human, to whom treatment including prophylactic treatment is provided. This includes human and non-human animals.

“Treat” or “treatment” refers to a therapeutic treatment wherein the object is to eliminate or lessen symptoms. Beneficial or desired clinical results include, but are not limited to, elimination of symptoms, alleviation of symptoms, diminishment of extent of a condition, stabilization (i.e., not worsening) of a condition's state, and delaying or slowing of progression of a condition.

A “therapeutic effect” may occur if there is a change in the condition being treated. There is a “change” in the condition being treated if there is at least 10% improvement, preferably at least 25%, more preferably at least 50%, even more preferably at least 75%, and most preferably 100%. The change can be based on improvements in the severity of the treated condition in an individual, or on a difference in the frequency of improved conditions in populations of individuals with and without treatment with the therapeutic compositions with which the compositions of the present invention are administered in combination. Similarly, a method of the present disclosure may comprise administering to a subject an amount of microvesicles (e.g., exosomes loaded with siRNA) that is “therapeutically effective”. The term “therapeutically effective” should be understood to have a definition corresponding to ‘having a therapeutic effect’.

The term “cancer” and its grammatical equivalents as used herein can refer to a hyperproliferation of cells whose unique trait—loss of normal controls—results in unregulated growth, lack of differentiation, local tissue invasion, and metastasis. With respect to the inventive methods, the cancer can be any cancer, including any of acute lymphocytic cancer, acute myeloid leukemia, alveolar rhabdomyosarcoma, bladder cancer, bone cancer, brain cancer, breast cancer, rectal cancer, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer, esophageal cancer, cervical cancer, fibrosarcoma, gastrointestinal carcinoid tumor, Hodgkin's lymphoma, hypopharynx cancer, kidney cancer, larynx cancer, leukemia, liquid tumors, liver cancer, lung cancer, lymphoma, malignant mesothelioma, mastocytoma, melanoma, multiple myeloma, nasopharynx cancer, non-Hodgkin lymphoma, ovarian cancer, pancreatic cancer, peritoneum, omentum, and mesentery cancer, pharynx cancer, prostate cancer, rectal cancer, renal cancer, skin cancer, small intestine cancer, soft tissue cancer, solid tumors, stomach cancer, testicular cancer, thyroid cancer, ureter cancer, and/or urinary bladder cancer. As used herein, the term “tumor” refers to an abnormal growth of cells or tissues, e.g., of malignant type or benign type.

“Tumor” refers to all neoplastic cell growth and proliferation, whether malignant or benign, and all pre-cancerous and cancerous cells and tissues.

“Inhibiting cancer or tumor progression” means inhibiting the development, growth, proliferation, or spreading of a tumor, including, but not limited to: inhibition of growth of cells in a tumor; inhibition of tumor growth; reduction in the number of tumor cells; reduction in tumor size; inhibition of tumor cell infiltration into adjacent peripheral organs and/or tissues; inhibition of metastasis; increased length of survival of a patient following treatment; and/or decreased mortality of a patient at a given time point following treatment.

“Inhibiting cancer or tumor cell growth or proliferation” means decreasing a cancer or tumor cell's growth or proliferation by at least 10%, 20%, 30%, 40%, 50%, 60%, 70%, 80%, 90%, 95%, or 100%, and includes inducing cell death in a cell or cells within a tumor.

The term “cancer neo-antigen” or “neo-antigen” or “neo-epitope” and its grammatical equivalents as used herein can refer to antigens that are not encoded in a normal, non-mutated host genome. A “neo-antigen” can in some instances represent either oncogenic viral proteins or abnormal proteins that arise as a consequence of somatic mutations. For example, aneo-antigen can arise by the disruption of cellular mechanisms through the activity of viral proteins. Another example can be an exposure of a carcinogenic compound, which in some cases can lead to a somatic mutation. This somatic mutation can ultimately lead to the formation of a tumor/cancer.

The term “cytotoxicity” as used in this specification, refers to an unintended or undesirable alteration in the normal state of a cell. The normal state of a cell may refer to a state that is manifested or exists prior to the cell's exposure to a cytotoxic composition, agent and/or condition. Generally, a cell that is in a normal state is one that is in homeostasis. An unintended or undesirable alteration in the normal state of a cell can be manifested in the form of, for example, cell death (e.g., programmed cell death), a decrease in replicative potential, a decrease in cellular integrity such as membrane integrity, a decrease in metabolic activity, a decrease in developmental capability, or any of the cytotoxic effects disclosed in the present application.

The term “T cell” and its grammatical equivalents as used herein can refer to a T cell from any origin. For example, a T cell can be a primary T cell, e.g., an autologous T cell, a cell line, etc. The T cell can also be human or non-human.

The term “TIL” or tumor infiltrating lymphocyte and its grammatical equivalents as used herein can refer to a cell isolated from a tumor. For example, a TIL can be a cell that has migrated to a tumor. A TIL can also be a cell that has infiltrated a tumor. A TIL can be any cell found within a tumor. For example, a TIL can be a T cell, B cell, monocyte, natural killer cell, or any combination thereof. A TIL can be a mixed population of cells. A population of TILs can comprise cells of different phenotypes, cells of different degrees of differentiation, cells of different lineages, or any combination thereof.

“Chimeric antigen receptor” or “CAR” or “CARs” as used herein refers to engineered receptors, which graft an antigen specificity onto cells (for example T cells such as naive T cells, central memory T cells, effector memory T cells or combination thereof). CARs are also known as artificial T-cell receptors, chimeric T-cell receptors or chimeric immunoreceptor.

### II. EXTRACELLULAR VESICLES SUCH AS EXOSOMES

Compositions and methods of the invention are directed to assaying one or more vesicles. Vesicles include without limitation the following types or species: extracellular vesicles (EVs), microvesicle, exosome, nanovesicle, dexosome, bleb, blebby, prostasome, microparticle, intralumenal vesicle, membrane fragment, intralumenal endosomal vesicle, endosomal-like vesicle, exocytosis vehicle, endosome vesicle, endosomal vesicle, apoptotic body, multivesicular body, secretory vesicle, phopholipid vesicle, liposomal vesicle, argosome, texasome, secresome, tolerosome, melanosome, oncosome, or exocytosed vehicle. Unless otherwise specified, methods that make use of a species of vesicle can be applied to other types of vesicles. Throughout this specification, references to an exosome can refer to other types of vesicles/extracellular vesicles.

In some embodiments, an EV such as an exosome is about 20 nm to about 200 nm in diameter. In some embodiments, an exosome is about 30 nm to about 190 nm or about 25 nm to about 180 nm in size. In some embodiments, an exosome is about 30 nm to about 170 nm in size. In some embodiments, an exosome is about 40 nm to about 160 nm in size. In some embodiments, an exosome is about 50 nm to about 150 or about 60 to about 140 nm, about 70 to about 130, about 80 to about 120, or about 90 to about 110 nm in diameter. In some embodiments, an exosome is about 20, 25, 30, 35, 50, 75, 100, 110, 125, or 150 nm in diameter. In some embodiments, an average exosome size in an exosomal composition or plurality of exosomes isolated or derived from a CAR T-cell is about 20, about 25, about 30, about 35, about 50, about 75, about 100, about 110, about 125, or about 150 nm; or about 20 to about 200, about 25 to about 250, about 30 to about 180, about 40 to about 170, about 50 to about 160, about 50 nm to about 150, about 60 to about 140 nm, about 70 to about 130, about 80 to about 120, or about 90 to about 110 nm in average diameter.

### A. Methods of Isolating Vesicles

Methods for isolating EVs such as exosomes from CAR T-cells are known in the art. See, e.g., “Method For Preparing Chimeric Antigen Receptor (Car)-Carrying Exosomes Derived From Immune Cells, And Use Of Car-Carrying Exosomes,” U.S. patent application Ser. No. 17/043,670 (Publication No. 2021/0030801) (Pharchoice Therapeutics, Inc. (Shanghai, CN)). “Cd19 Chimeric Antigen Receptor-Exosome Targets And Induces Cytotoxicity In Cd19 Positive B-Cell Malignancies,” WIPO Patent Application No. PCT/US2021/061700 (Publication No. WO/2022/120107) (Feinstein Institutes For Medical Research (US)). “Tailored Hypoimmunenanovesicular Delivery Systems For Cancer Tumors, Hereditary And Infectious Diseases,” U.S. patent application Ser. No. 18/571,007 (Publication No. 20240307317)

For example, in some embodiments, the CAR T-cell-derived vesicles are isolated using size exclusion chromatography, PEG-precipitation of the vesicles, filtration, or immunosorbent capture. In some embodiments, isolating the vesicles comprises using an agarose-based gel. Size exclusion chromatography, PEG-precipitation, filtration, and immunosorbent capture techniques are known in the art.

In some embodiments, a void volume fraction is isolated and comprises the vesicles of interest. Further, in some embodiments, the CAR T-cell-derived vesicles can be further isolated after chromatographic separation by centrifugation techniques (of one or more chromatography fractions), as is generally known in the art. In some embodiments, for example, density gradient centrifugation can be used to further isolate the vesicles. Still further, in some embodiments, it can be desirable to further separate the CAR T-cell-derived isolated vesicles from vesicles of other origin.

In some embodiments, CAR T-cell-derived vesicles are isolated using affinity selection. For example, CAR T-cell-derived vesicles can be isolated based on their affinity for particular binding agents. For example, a binding agent can be an antibody or an aptamer. Thus, binding agents can be used in affinity selection to select particular ligands, molecules, substances, or the like based on the extent to which they bind with a particular binding agent. In some embodiments, affinity selection comprises separating the CAR T-cell-derived vesicles from non-CAR T-cell-derived EVs by immunosorbent capture using an anti-cancer antigen antibody as the binding agent.

In some embodiments, EVs are isolated from cellular preparations by methods comprising one or more of filtration, centrifugation, antigen-based capture and the like. In some embodiments, a population of cells grown in culture are collected and pooled. In some embodiments, monolayers of cells are pooled. In some embodiments, cells grown in suspension are used. In some embodiments, the pooled population is subject to one or more rounds of centrifugation e.g., ultracentrifugation and/or density centrifugation to separate the EV fraction from cells and cellular debris. In some embodiments, centrifugation need not be performed to harvest EVs. In some embodiments, size exclusion filtration is used in conjunction with, or in place of centrifugation, in order to collect a particular size (e.g., diameter) of EV. In some embodiments, filtration need not be used. In some embodiments, EVs are captured by affinity chromatography using agents that bind to unique markers on or in the EVs (e.g., transmembrane proteins)). In such instances, the unique markers can be used to selectively enrich a particular EV population, such as those derived from cancer cells.

In one aspect, a method of isolating EVs comprises centrifuging a cell medium at low speed. For example, 200 mL, of cellular medium can be centrifuged at 300 g for 10 min. The method can further comprise removing a cell pellet after the centrifugation at low speed. The method can further comprise centrifuging the supernatant of the low speed centrifugation step at a medium speed. For example, the supernatant can be centrifuged at 2,000 g for 10 min. The method can further comprise removing a pellet of cell debris and apoptotic bodies that result from the second centrifugation step. The method can further comprise centrifuging the supernatant of the second centrifugation step at high speed. For example, the supernatant can be centrifuged at 10,000 g for 30 min. The method can further comprise removing a pellet containing resulting from the high speed centrifugation step. The method can further comprise filtering the supernatant of the high speed centrifugation step with a membrane. For example, the supernatant of the high speed centrifugation step can be filtered with a Centricon Plus 70-100 KD (10 nm pore size approx.) centrifugal filter at 3500 g for 15 min The method can further comprise collecting a retentate of the filtering step that is enriched in EVs, such as exosomes. The retentate can be resuspended in a volume of buffer. For example, the volume of the retentate can be resuspended in 500 μL of PBS. In some embodiments, the filtrate of the filtering step can be discarded. The volume of the filtration residue was made to 500 μL using PBS.

### B. Loading EVs Such as Exosomes

Methods for loading exosomes are known in the art. See, e.g., U.S. patent application Ser. No. 16/194,230 (Publication No. US2019/0151456) (“Compositions of Engineered Exosome and Methods of Loading Luminal Exosome Payloads”); U.S. patent application Ser. No. 16/239,052 (Publication No. 2020/0215201) (“Exosome Vessels for Delivery of Molecular Cargo”); Zeng et al., 12(10) CELLS 1416 (2023) (“Current Strategies for Exosome Cargo Loading and Targeted Delivery”); U.S. Pat. No. 10,758,486 (Engineered Exosomes for the Delivery of Bioactive Cargo Using Transmembrane VSV-G”); Canadian Patent No. 2997811 (“Exosome Packaging of Nucleic Acids”); and European Patent No. 3706796 (“Exosomes Comprising RNA Therapeutics”).

Thus, methods for loading exosome with cargo such as siRNA include, but are not limited to, electroporation, transfection, lipofection, microinjection, and calcium phosphate mediated transfection.

In certain embodiments, exosomes loaded with cargo are considered mature exosomes and are inspected for cGMP compliance, purity and stability for quality assurance and quality check. Next, mature exosomes that have passed the quality check undergo an expansion process if needed. Next, the mature exosomes are diluted and premix into saline/vehicle (depending on the characteristics of the load) for a ready to administer tube/cartridge. Finally, the suspension is frozen and stored or shipped to a site for use in clinical or preclinical studies and to patients for self-injection of approved-clinical grade mature exosomes.

### III. RNA INTERFERENCE COMPOSITIONS FOR TARGETING TTC3, EPCAM AND/OR PCA3 MRNA, AS WELL AS GPX4, ACSL4, HLX, MYC AND/OR IGF2BP3

In one aspect of the present invention, the expression of TTC3, EpCAM and/or PCA3, as well as GPX4, ACSL4, HLX, MYC and/or IGF2BP3, may be inhibited by the use of RNA interference techniques (RNAi). RNAi is a remarkably efficient process whereby double-stranded RNA (dsRNA) induces the sequence-specific degradation of homologous mRNA in animals and plant cells. See Hutvagner and Zamore, 12 CURR. OPIN. GENET. DEV. 225-32 (2002); Hammond et al., 2 NATURE REV. GEN. 110-19 (2001); Sharp, 15 GENES DEV. 485-90 (2001). RNAi can be triggered, for example, by nucleotide (nt) duplexes of small interfering RNA (siRNA) (Chiu et al., 10 MOL. CELL. 549-61 (2002); Elbashir et al., 411 Nature 494-98 (2001)), micro-RNAs (miRNA), functional small-hairpin RNA (shRNA), or other dsRNAs which are expressed in-vivo using DNA templates with RNA polymerase III promoters. See, e.g., Zeng et al., 9 MOL. CELL. 1327-33 (2002); Paddison et al., 16 GENES DEV. 948-58 (2002); Lee et al., 20 NATURE BIOTECHNOL. 500-05 (2002); Paul et al., 20 NATURE BIOTECHNOL. 505-08 (2002); Tuschl, 20 NATURE BIOTECHNOL. 440-48 (2002); Yu et al., 99(9) PROC. NATL. ACAD. SCI. USA, 6047-52 (2002); McManus et al., 8 RNA 842-50 (2002); Sui et al., 99(6) PROC. NATL. ACAD. SCI. USA 5515-20 (2002).

As used herein, a TTC3, EpCAM and/or PCA3, as well as GPX4, ACSL4, HLX, MYC and/or IGF2BP3, inhibitory nucleic acid sequence can be a siRNA sequence or a miRNA sequence. An approximately 21-25 nucleotide siRNA or miRNA sequence can, for example, be produced from an expression vector by transcription of a short-hairpin RNA (shRNA) sequence, a 60-80 nucleotide precursor sequence, which is processed by the cellular RNAi machinery to produce either an siRNA or miRNA sequence. Alternatively, an approximately 21-25 nucleotide siRNA or miRNA sequence can, for example, be synthesized chemically. Chemical synthesis of siRNA or miRNA sequences is commercially available from such corporations as Dharmacon, Inc. (Lafayette, Colo.), Qiagen (Valencia, Calif.), and Ambion, Inc. (Austin, Tex.). In certain embodiments, an siRNA sequence binds a unique sequence within the TTC3, EpCAM and/or PCA3 mRNA with exact complementarity and results in the degradation of the mRNA molecule. An siRNA sequence can bind anywhere within the mRNA molecule. In some embodiments, an miRNA sequence binds a unique sequence within the mRNA with exact or less than exact complementarity and results in the translational repression of the mRNA molecule. An miRNA sequence can bind anywhere within the mRNA molecule, but preferably binds within the 3′UTR of the mRNA molecule. Methods of delivering siRNA or miRNA molecules are known in the art. See, e.g., Oh and Park, Adv. Drug Deliv. Rev. 61(10):850-62 (2009); Gondi and Rao, J. Cell. Physiol. 220(2):285-91 (2009); and Whitehead et al., Nat. Rev. Drug Discov. 8(2)129-38 (2009).

### A. Small Interfering RNA

In particular embodiments, the present invention features “small interfering RNA molecules” (“siRNA molecules” or “siRNA”), methods of making siRNA molecules and methods for using siRNA molecules (e.g., therapeutic methods). The siRNAs of this invention encompass any siRNAs that can modulate the selective degradation of TTC3, EpCAM and/or PCA3 mRNA, as well as GPX4, ACSL4, HLX, MYC and/or IGF2BP3.

In a specific embodiment, the siRNA of the present invention may comprise double-stranded small interfering RNA molecules (ds-siRNA). A ds-siRNA molecule of the present invention may be a duplex made up of a sense strand and a complementary antisense strand, the antisense strand being sufficiently complementary to a target mRNA to mediate RNAi. The siRNA molecule may comprise about 10 to about 50 or more nucleotides. More specifically, the siRNA molecule may comprise about 16 to about 30, e.g., 16, 17, 18, 19, 20, 21, 22, 23, 24, 25, 26, 27, 28, 29, or 30 nucleotides in each strand. The strands may be aligned such that there are at least 1, 2, or 3 bases at the end of the strands which do not align (e.g., for which no complementary bases occur in the opposing strand) such that an overhang of 1, 2 or 3 residues occurs at one or both ends of the duplex when strands are annealed.

In an alternative embodiment, the siRNA of the present invention may comprise single-stranded small interfering RNA molecules (ss-siRNA). Similar to the ds-siRNA molecules, the ss-siRNA molecule may comprise about 10 to about 50 or more nucleotides. More specifically, the ss-siRNA molecule may comprise about 15 to about 45 or more nucleotides. Alternatively, the ss-siRNA molecule may comprise about 19 to about 40 nucleotides. The ss-siRNA molecules of the present invention comprise a sequence that is “sufficiently complementary” to a target mRNA sequence to direct target-specific RNA interference (RNAi), as defined herein, e.g., the ss-siRNA has a sequence sufficient to trigger the destruction of the target mRNA by the RNAi machinery or process. In one embodiment, the ss-siRNA molecule can be designed such that every residue is complementary to a residue in the target molecule. Alternatively, substitutions can be made within the molecule to increase stability and/or enhance processing activity of the molecule. Substitutions can be made within the strand or can be made to residues at the ends of the strand. In a specific embodiment, the 5′-terminus may be phosphorylated (e.g., comprises a phosphate, diphosphate, or triphosphate group). In another embodiment, the 3′ end of an siRNA may be a hydroxyl group in order to facilitate RNAi, as there is no requirement for a 3′ hydroxyl group when the active agent is a ss-siRNA molecule. In other instances, the 3′ end (e.g., C3 of the 3′ sugar) of ss-siRNA molecule may lack a hydroxyl group (e.g., ss-siRNA molecules lacking a 3′ hydroxyl or C3 hydroxyl on the 3′ sugar (e.g., ribose or deoxyribose).

In another aspect, the siRNA molecules of the present invention may be modified to improve stability under in vitro and/or in vivo conditions, including, for example, in serum and in growth medium for cell cultures. In order to enhance the stability, the 3′-residues may be stabilized against degradation, e.g., they may be selected such that they consist of purine nucleotides, particularly adenosine or guanosine nucleotides. Alternatively, substitution of pyrimidine nucleotides by modified analogues, e.g., substitution of uridine by 2′-deoxythymidine is tolerated and does not affect the efficiency of RNA interference. For example, the absence of a 2′ hydroxyl may significantly enhance the nuclease resistance of the siRNAs in tissue culture medium.

Furthermore, the siRNAs of the present invention may include modifications to the sugar-phosphate backbone or nucleosides. These modifications can be tailored to promote selective genetic inhibition, while avoiding a general panic response reported to be generated by siRNA in some cells. In addition, modifications can be introduced in the bases to protect siRNAs from the action of one or more endogenous enzymes.

In an embodiment of the present invention, the siRNA molecule may contain at least one modified nucleotide analogue. The nucleotide analogues may be located at positions where the target-specific activity, e.g., the RNAi mediating activity is not substantially effected, e.g., in a region at the 5′-end and/or the 3′-end of the RNA molecule. Particularly, the ends may be stabilized by incorporating modified nucleotide analogues. Examples of nucleotide analogues include sugar- and/or backbone-modified ribonucleotides (e.g., include modifications to the phosphate-sugar backbone). For example, the phosphodiester linkages of natural RNA may be modified to include at least one of a nitrogen or sulfur heteroatom. In backbone-modified ribonucleotides, the phosphoester group connecting to adjacent ribonucleotides may be replaced by a modified group, e.g., a phosphothioate group. In sugar-modified ribonucleotides, the 2′ OH-group may be replaced by a group selected from H, OR, R, halo, SH, SR, NH₂, NHR, NR₂ or ON, wherein R is C₁-C₆ alkyl, alkenyl or alkynyl and halo is F, Cl, Br or I.

Nucleobase-modified ribonucleotides may also be utilized, e.g., ribonucleotides containing at least one non-naturally occurring nucleobase instead of a naturally occurring nucleobase. Bases may be modified to block the activity of adenosine deaminase. Exemplary modified nucleobases include, but are not limited to, uridine and/or cytidine modified at the 5-position, e.g., 5-(2-amino)propyl uridine, 5-bromo uridine; adenosine and/or guanosines modified at the 8 position, e.g., 8-bromo guanosine; deaza nucleotides, e.g., 7-deaza-adenosine; O- and N-alkylated nucleotides, e.g., N6-methyl adenosine are suitable. It should be noted that the above modifications may be combined.

Derivatives of siRNAs may also be utilized herein. For example, cross-linking can be employed to alter the pharmacokinetics of the composition, e.g., to increase half-life in the body. Thus, the present invention includes siRNA derivatives that include siRNA having two complementary strands of nucleic acid, such that the two strands are crosslinked. The present invention also includes siRNA derivatives having a non-nucleic acid moiety conjugated to its 3′ terminus (e.g., a peptide), organic compositions (e.g., a dye), or the like. Modifying siRNA derivatives in this way may improve cellular uptake or enhance cellular targeting activities of the resulting siRNA derivative as compared to the corresponding siRNA, are useful for tracing the siRNA derivative in the cell, or improve the stability of the siRNA derivative compared to the corresponding siRNA.

The siRNAs of the present invention can be enzymatically produced or totally or partially synthesized. Moreover, the siRNAs can be synthesized in vivo or in vitro. For siRNAs that are biologically synthesized, an endogenous or a cloned exogenous RNA polymerase may be used for transcription in vivo, and a cloned RNA polymerase can be used in vitro. siRNAs that are chemically or enzymatically synthesized are preferably purified prior to the introduction into the cell.

Although one hundred percent (100%) sequence identity between the siRNA and the target region is preferred in particular embodiments, it is not required to practice the invention. siRNA molecules that contain some degree of modification in the sequence can also be adequately used for the purpose of this invention. Such modifications may include, but are not limited to, mutations, deletions or insertions, whether spontaneously occurring or intentionally introduced.

Moreover, not all positions of a siRNA contribute equally to target recognition. In certain embodiments, for example, mismatches in the center of the siRNA may be critical and could essentially abolish target RNA cleavage. In other embodiments, the 3′ nucleotides of the siRNA do not contribute significantly to specificity of the target recognition. In particular, residues 3′ of the siRNA sequence which is complementary to the target RNA (e.g., the guide sequence) may not be critical for target RNA cleavage.

Sequence identity may be determined by sequence comparison and alignment algorithms known to those of ordinary skill in the art. To determine the percent identity of two nucleic acid sequences (or of two amino acid sequences), the sequences are aligned for optimal comparison purposes (e.g., gaps can be introduced in the first sequence or second sequence for optimal alignment). The nucleotides (or amino acid residues) at corresponding nucleotide (or amino acid) positions are then compared. When a position in the first sequence is occupied by the same residue as the corresponding position in the second sequence, then the molecules are identical at that position. The percent identity between the two sequences is a function of the number of identical positions shared by the sequences (e.g., % homology=# of identical positions/total # of positions×100), optionally penalizing the score for the number of gaps introduced and/or length of gaps introduced.

The comparison of sequences and determination of percent identity between two sequences can be accomplished using a mathematical algorithm. In one embodiment, the alignment generated over a certain portion of the sequence aligned having sufficient identity but not over portions having low degree of identity (e.g., a local alignment). A non-limiting example of a local alignment algorithm utilized for the comparison of sequences is the algorithm of Karlin and Altschul, 87 PROC. NATL. ACAD. SCI. USA 2264-68 (1990), and as modified as in Karlin and Altschul 90 PROC. NATL. ACAD. SCI. USA 5873-77 (1993). Such an algorithm is incorporated into the BLAST programs (version 2.0) of Altschul, et al., 215 J. MOL. BIOL. 403-10 (1990).

In another embodiment, the alignment may be optimized by introducing appropriate gaps and determining percent identity over the length of the aligned sequences (e.g., a gapped alignment). To obtain gapped alignments for comparison purposes, Gapped BLAST can be utilized as described in Altschul et al., 25(17) NUCLEIC ACIDS RES. 3389-3402 (1997). In another embodiment, the alignment may be optimized by introducing appropriate gaps and determining percent identity over the entire length of the sequences aligned (e.g., a global alignment). A non-limiting example of a mathematical algorithm utilized for the global comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

In particular embodiments, greater than 90% sequence identity, e.g., 91%, 92%, 93%, 94%, 95%, 96%, 97%, 98%, 99% or even 100% sequence identity, between the siRNA and the portion of the target gene may be used. Alternatively, the siRNA may be defined functionally as a nucleotide sequence (or oligonucleotide sequence) that is capable of hybridizing with a portion of the target gene transcript (e.g., 400 mM NaCl, 40 mM PIPES pH 6.4, 1 mM EDTA, 50 °C or 70 °C hybridization for 12-16 hours; followed by washing). Additional hybridization conditions include, but are not limited to, hybridization at 70 °C in 1×SSC or 50 °C in 1×SSC, 50% formamide followed by washing at 70 °C in 0.3×SSC or hybridization at 70 °C in 4×SSC or 50 °C in 4×SSC, 50% formamide followed by washing at 67 °C in 1×SSC. The hybridization temperature for hybrids anticipated to be less than 50 base pairs in length can be about 5-10 °C less than the melting temperature (Tm) of the hybrid, where Tm is determined according to the following equations. For hybrids less than 18 base pairs in length, Tm(°C)=2(# of A+T bases)+4(# of G+C bases). For hybrids between 18 and 49 base pairs in length, Tm(°C)=81.5+16.6(log 10[Na⁺])+0.41(% G+C)−(600/N), where N is the number of bases in the hybrid, and [Na⁺] is the concentration of sodium ions in the hybridization buffer ([Na⁺] for 1×SSC=0.165 M). Additional examples of stringency conditions for polynucleotide hybridization are provided in Sambrook, J., E. F. Fritsch, and T. Maniatis, 1989, Molecular Cloning: A Laboratory Manual, Cold Spring Harbor Laboratory Press, Cold Spring Harbor, N.Y., chapters 9 and 11, and Current Protocols in Molecular Biology, 1995, F. M. Ausubel et al., eds., John Wiley & Sons, Inc., sections 2.10 and 6.3-6.4, incorporated herein by reference. The length of the identical nucleotide sequences may be at least about 10, 12, 15, 17, 20, 22, 25, 27, 30, 32, 35, 37, 40, 42, 45, 47 50 or more bases.

B. Other Compositions for Targeting TTC3, EpCAM and/or PCA3 DNA or mRNA, as well as GPX4, ACSL4, HLX, MYC and/or IGF2BP3 DNA or mRNA

Antisense molecules can act in various stages of transcription, splicing and translation to block the expression of a target gene. Without being limited by theory, antisense molecules can inhibit the expression of a target gene by inhibiting transcription initiation by forming a triple strand, inhibiting transcription initiation by forming a hybrid at an RNA polymerase binding site, impeding transcription by hybridizing with an RNA molecule being synthesized, repressing splicing by hybridizing at the junction of an exon and an intron or at the spliceosome formation site, blocking the translocation of an mRNA from nucleus to cytoplasm by hybridization, repressing translation by hybridizing at the translation initiation factor binding site or ribosome biding site, inhibiting peptide chain elongation by hybridizing with the coding region or polysome binding site of an mRNA, or repressing gene expression by hybridizing at the sites of interaction between nucleic acids and proteins. An example of an antisense oligonucleotide of the present invention is a cDNA that, when introduced into a cell, transcribes into an RNA molecule having a sequence complementary to at least part of the TTC3, EpCAM and/or PCA3 mRNA, as well as GPX4, ACSL4, HLX, MYC and/or IGF2BP3mRNA.

Thus, as used herein, in other embodiments, a TTC3, EpCAM and/or PCA3, as well as GPX4, ACSL4, HLX, MYC and/or IGF2BP3, inhibitory nucleic acid sequence can be an antisense nucleic acid sequence. Antisense nucleic acid sequences can, for example, be transcribed from an expression vector to produce an RNA which is complementary to at least a unique portion of the mRNA and/or the endogenous gene which encodes target protein. Hybridization of an antisense nucleic acid molecule under specific cellular conditions results in inhibition of the target protein expression by inhibiting transcription and/or translation.

Furthermore, antisense oligonucleotides of the present invention include oligonucleotides having modified sugar-phosphodiester backbones or other sugar linkages, which can provide stability against endonuclease attacks. The present invention also encompasses antisense oligonucleotides that are covalently attached to an organic or other moiety that increase their affinity for a target nucleic acid sequence. For example, intercalating agents, alkylating agents, and metal complexes can be also attached to the antisense oligonucleotides of the present invention to modify their binding specificities.

In other embodiments, the present invention provides ribozymes as a tool to inhibit TTC3, EpCAM and/or PCA3 expression, as well as GPX4, ACSL4, HLX, MYC and/or IGF2BP3 expression. Ribozymes are enzymatic RNA molecules capable of catalyzing the specific cleavage of RNA. The characteristics of ribozymes are well-known in the art. See, e.g., Rossi, 4 CURRENT BIOLOGY 469-71 (1994). Without being limited by theory, the mechanism of ribozyme action involves sequence specific hybridization of the ribozyme molecule to complementary target RNA, followed by an endonucleolytic cleavage. In particular embodiments, the ribozyme molecules include one or more sequences complementary to the target gene mRNA, and include the well-known catalytic sequence responsible for mRNA cleavage. See U.S. Pat. No. 5,093,246. Using the known sequence of the target mRNA, a restriction enzyme-like ribozyme can be prepared using standard techniques.

The expression of the TTC3, EpCAM and/or PCA3 genes, as well as GPX4, ACSL4, HLX, MYC and/or IGF2BP3 genes, can also be inhibited by using triple helix formation. Nucleic acid molecules to be used in triple helix formation for the inhibition of transcription can be single stranded and composed of deoxynucleotides. The base composition of these oligonucleotides must be designed to promote triple helix formation via Hoogsteen base paring rules, which generally require sizeable stretches of either purines or pyrimidines to be present on one strand of a duplex. Nucleotide sequences may be pyrimidine-based, which will result in TAT and CGC⁺ triplets across the three associated strands of the resulting triple helix. The pyrimidine-rich molecules provide base complementarity to a purine-rich region of a single strand of the duplex in a parallel orientation to that strand. In addition, nucleic acid molecules that are purine-rich, e.g., containing a stretch of G residues, may be chosen. These molecules will form a triple helix with a DNA duplex that is rich in GC pairs, in which the majority of the purine residues are located on a single strand of the targeted duplex, resulting in GGC triplets across the three strands in the triplex.

Alternatively, the potential sequences that can be targeted for triple helix formation may be increased by creating a so-called “switchback” nucleic acid molecule. Switchback molecules are synthesized in an alternating 5′-3′,3′-5′ manner, such that they base pair first with one strand of a duplex and then the other, eliminating the necessity for a sizeable stretch of either purines or pyrimidines to be present on one strand of a duplex.

The expression of TTC3, EpCAM and/or PCA3, as well as GPX4, ACSL4, HLX, MYC and/or IGF2BP3, may be also inhibited by what is referred to as “co-repression.” Co-repression refers to the phenomenon in which, when a gene having an identical or similar to the target sequence is introduced to a cell, expression of both introduced and endogenous genes becomes repressed. This phenomenon, although first observed in plant system, has been observed in certain animal systems as well. The sequence of the gene to be introduced does not have to be identical to the target sequence, but sufficient homology allows the co-repression to occur. The determination of the extent of homology depends on individual cases, and is within the ordinary skill in the art.

It would be readily apparent to one of ordinary skill in the art that other methods of gene expression inhibition that selectively target a TTC3, EpCAM and/or PCA3 DNA or mRNA, as well as GPX4, ACSL4, HLX, MYC and/or IGF2BP3 DNA or mRNA, can also be used in connection with this invention without departing from the spirit of the invention. In a specific embodiment, using techniques known to those of ordinary skill in the art, the present invention contemplates affecting the promoter region of the target to effectively switch off transcription.

### C. Design and Production of the RNAi Compositions

One or more of the following guidelines may be used in designing the sequence of siRNA and other nucleic acids designed to bind to a target mRNA, e.g., shRNA, stRNA, antisense oligonucleotides, ribozymes, and the like, that are advantageously used in accordance with the present invention.

Beginning with the AUG start codon of the TTC3, EpCAM and/or PCA3 genes, as well as GPX4, ACSL4, HLX, MYC and/or IGF2BP3 genes, each AA dinucleotide sequence and the 3′ adjacent 16 or more nucleotides are potential siRNA targets. In a specific embodiment, the siRNA is specific for a target region that differs by at least one base pair between the wild type and mutant allele or between splice variants. In dsRNAi, the first strand is complementary to this sequence, and the other strand identical or substantially identical to the first strand. siRNAs with lower G/C content (35-55%) may be more active than those with G/C content higher than 55%. Thus in one embodiment, the invention includes nucleic acid molecules having 35-55% G/C content. In addition, the strands of the siRNA can be paired in such a way as to have a 3′ overhang of 1 to 4, e.g., 2, nucleotides. Thus in another embodiment, the nucleic acid molecules may have a 3′ overhang of 2 nucleotides, such as TT. The overhanging nucleotides may be either RNA or DNA. In one embodiment, it may be desirable to choose a target region wherein the mismatch is a purine:purine mismatch.

Using any method known in the art, compare the potential targets to the appropriate genome database (human, mouse, rat, etc.) and eliminate from consideration any target sequences with significant homology to other coding sequences. One such method for such sequence homology searches is known as BLAST, which is available at National Center for Biotechnology Information website (http://www.ncbi.nih.gov). Select one or more sequences that meet the criteria for evaluation.

Another method includes selecting in the sequence of the target mRNA, a region located from about 50 to about 100 nt 3′ from the start codon. In this region, search for the following sequences: AA(N19)TT or AA(N21), where N=any nucleotide. The GC content of the selected sequence should be from about 30% to about 70%, preferably about 50%. To maximize the specificity of the RNAi, it may be desirable to use the selected sequence in a search for related sequences in the genome of interest; sequences absent from other genes are preferred. The secondary structure of the target mRNA may be determined or predicted, and it may be preferable to select a region of the mRNA that has little or no secondary structure, but it should be noted that secondary structure seems to have little impact on RNAi. When possible, sequences that bind transcription and/or translation factors should be avoided, as they might competitively inhibit the binding of a siRNA, sbRNA or stRNA (as well as other antisense oligonucleotides) to the mRNA. Further general information about the design and use of siRNA may be found in “The siRNA User Guide,” available at The Max-Planck-Institut fur Biophysikalishe Chemie website (http://www.mpibpc.mpg.de).

Negative control siRNAs should have the same nucleotide composition as the selected siRNA, but without significant sequence complementarity to the appropriate genome. Such negative controls may be designed by randomly scrambling the nucleotide sequence of the selected siRNA; a homology search can be performed to ensure that the negative control lacks homology to any other gene in the appropriate genome.

D. Delivery of TTC3, EpCAM and/or PCA3 RNA, as well as GPX4, ACSL4, HLX, MYC and/or IGF2BP3, Targeting Compositions

In several embodiments, the RNA Targeting Compositions are encapsulated within a microvesicle such as a CAR-T exosome. However, in alternative embodiments, delivery of the compositions of the present invention (e.g., siRNAs, antisense oligonucleotides, or other compositions described herein) into a patient can either be direct, e.g., the patient is directly exposed to the compositions of the present invention or compound-carrying vector, or indirect, e.g., cells are first transformed with the compositions of this invention in vitro, then transplanted into the patient for cell replacement therapy. These two approaches are known as in vivo and ex vivo therapy, respectively.

In the case of in vivo therapy, the compositions of the present invention are directly administered in vivo, where they are expressed to produce the encoded product. This can be accomplished by any of numerous methods known in the art, e.g., by constructing them as part of an appropriate nucleic acid expression vector and administering them so that they become intracellular, by infection using a defective or attenuated retroviral or other viral vector, by direct injection of naked DNA, by coating with lipids or cell-surface receptors or transfecting agents, encapsulation in liposomes, nanoparticles, microparticles, or microcapsules, by administering them in linkage to a peptide which is known to enter the cell or nucleus, or by administering them in linkage to a ligand subject to receptor-mediated endocytosis which can be used to target cell types specifically expressing the receptors. Further, the compositions of the present invention can be targeted in vivo for cell specific uptake and expression, by targeting a specific receptor. See, e.g., WO93/14188, WO 93/20221, WO 92/22635, WO92/20316, and WO 92/06180.

Ex vivo therapy involves transferring the compositions of the present invention to cells in tissue culture by methods well-known in the art such as electroporation, transfection, lipofection, microinjection, calcium phosphate mediated transfection, cell fusion, chromosome-mediated gene transfer, microcell-mediated gene transfer, spheroplast fusion, and infection with a viral vector containing the nucleic acid sequences. These techniques should provide for the stable transfer of the compositions of this invention to the cell, so that they are expressible by the cell and preferably heritable and expressible by its cell progeny. In particular embodiments, the method of transfer includes the transfer of a selectable marker to the cells. The cells are then placed under selection to isolate those cells that have taken up and are expressing the transferred compositions. The resulting recombinant cells can be delivered to a patient by various methods known in the art. Examples of the delivery methods include, but are not limited to, subcutaneous injection, skin graft, and intravenous injection.

E. Examples of siRNA

Examples of EpCAM siRNA include, but are not limited to, sense, 5′-CAATGCCAGTGTACTTCAGTT-3′ (SEQ ID NO:1) and antisense, 5′-AACTGAAGTACACTGGCATTG-3′ (SEQ ID NO:2) (See Qi et al., 47(5) TURK. J. BIOCHEM. 620-25 (2022) (“Epcam Is Critical For Tumor Proliferation And Oxaliplatin Chemoresistance In Epcam^{high}/CD44⁺ Colorectal Cancer Stem Cells”); and 5′-UGCCAGUGUACUUCAGUUG-3′ (SEQ ID NO:3) (See Massoner et al., 111(5) BR. J. CANCER 955-64 (2014) (“Epcam Is Overexpressed In Local And Metastatic Prostate Cancer, Suppressed By Chemotherapy And Modulated By MET-Associated Mirna-200c/205”). The mRNA sequence of EpCAM is shown in SEQ ID NO:4. The coding sequence is shown in nucleotides 196-1140 of SEQ ID NO:4. The amino acid sequence of EPCAM is shown in SEQ ID NO:5.

Examples of PCA3 siRNA include, but are not limited to, 5′-GCAGAAGCCAGAAUUUGAAUUCCCT-3′ (SEQ ID NO:6), 5′-CUAGCACACAGCAUGAUCAUUACGG-3′ (SEQ ID NO:7), and 5′-CCACAAUAUGCAUAAAUCUAACUCC-3′ (SEQ ID NO:8) (See Ferreira et al., 12 BMC CANCER 507 (2012) (“PCA3 noncoding RNA is involved in the control of prostate-cancer cell survival and modulates androgen receptor signaling”) and Ozgur et al., 37(7) ANTICANCER RES. 3631-37 (2017) (“PCA3 Silencing Sensitizes Prostate Cancer Cells to Enzalutamide-mediated Androgen Receptor Blockade”)) (See also IDT Technologies, Coralville, IA, USA). The RNA sequence of PCA3 is shown in SEQ ID NO:9 (NCBI Reference Sequence: NR_132312.2).

Examples of TTC3 siRNA include, but are not limited to, 5′-GCACCUAUAUUUACUACUUTT-3′ (SEQ ID NO:10), and 5′-AAGUAGUAAAUAUAGGUGCTG-3′ (SEQ ID NO: 11). The mRNA sequence of TTC3 is shown in SEQ ID NO:12. The coding sequence is shown in nucleotides 123-6200 of SEQ ID NO:12. The amino acid sequence of TTC3 is shown in SEQ ID NO:13.

Examples of ACSL4 siRNA include, but are not limited to, 5′-GGAUAUUCUUCUCCGCUUAUU-3′ (SEQ ID NO:14), and 5′-UAAGCGGAGAAGAAUAUCCAA-3″ (SEQ ID NO:15). The mRNA sequence of ACSL4 is shown in SEQ ID NO:16. The coding sequence is shown in nucleotides 318-2330 of SEQ ID NO:16. The amino acid sequence of ACSL4 is shown in SEQ ID NO:17.

Examples of GPX4 siRNA include, but are not limited to, 5′-AAUUCGAUAUGUUCAGCAAUU-3′ (SEQ ID NO:18) and 5′-UUGCUGAACAUAUCGAAUUUG-3′ (SEQ ID NO:19. GPX4 siRNA can further include 5′-GGUGCUUCGUCUGAAUCAU-3′ (SEQ ID NO:20), 5′-AUGAUUCAGACGAAGCACC-3′ (SEQ ID NO:21), 5′-GCGCAGAUCAAAGCCUUUG-3′ (SEQ ID NO:22), and 5-′CAAAGGCUUUGAUCUGCGC-3′ (SEQ ID NO:23). The mRNA sequence of GPX4 is shown in SEQ ID NO:24. The coding sequence is shown in nucleotides 51-734 of SEQ ID NO:24. The amino acid sequence of GPX4 is also shown in SEQ ID NO:25.

Examples of HLX siRNA include, but are not limited to, 5′-CCCUUAAACUCGAACCCAAUU-3′ (SEQ ID NO: 26) (including a modified version 5′-rCrCrCrUrUrArArArCrUrCrGrArArCrCrCrArArUrU-3′) and 5′-UUGGGUUCGAGUUUAAGGGAC-3′ (SEQ ID NO: 27) (including a modified version 5′-rUrUrGrGrGrUrUrCrGrArGrUrUrUrArArGrGrGrArC-3′). An HLX siRNA can also comprise; 5′-UGAAUUUCUUGGGUUCGAGTT-3′ (SEQ ID NO:28) or 5′-AAACCUUUUCUCCAGGCCUTT-3′ (SEQ ID NO:29). See Rajaraman et al., 176(1) Am. J. Pathol. 278-87 (2010). In further embodiments, an HLX siRNA can comprise 5′-UGAAUUUCUUGGGUUCGAGTT-3′ (SEQ ID NO:30). See Rajamaran et al., 83(4) Biol. Reproduction 676-83 (2010). The mRNA sequence of HLX (NCBI Reference Sequence: NM_021958.4) is shown in SEQ ID NO:31. The coding sequence is shown in nucleotides 416-1882 of SEQ ID NO:31. The amino acid sequence of HLX is shown in SEQ ID NO:32.

Examples of MYC siRNA include 5′-GCTTGTACCTGCAGGATCT-3′ (targeting exon 2) (SEQ ID NO:33) and 5′-CGTCCAAGCAGAGGAGCAA-3′ (targeting exon 3) (SEQ ID NO:34). See Reyes-Gonzalez et al., 14(10) MOL. CANCER THER. 2260-69 (2015). A MYC siRNA can also comprise 5′-GCCACAGCAUACAUCCUGUUU-3′ (SEQ ID NO: 35) or 5′-ACAGGAUGUAUGCUGUGGCUU-3′ (SEQ ID NO:36). See Zhang et al., 14 CELL. MOL. Biol. Letters 305-18 (2009). The mRNA sequence of MYC (NCBI Reference Sequence: NM_002467.6) is shown in SEQ ID NO:37. The coding sequence is shown in nucleotides 364-1728 of SEQ ID NO:37. The amino acid sequence of MYC is shown in SEQ ID NO:38.

Examples of IGF2BP3 siRNA include 5′-CGGUGAAUGAACUUCAGAATT-3′ (SEQ ID NO:39) and 5′-GCUGCUGAGAAGUCGAUUATT-3′ (SEQ ID NO:40) (see Huang et al., 81(17) CELL. MOL. LIFE SCI. 1-14 (2024)), as well as 5′-GCAAAGGATTCGGAAACTT-3′ (SEQ ID NO:41), 5′-GGTGAAACTTGAAGCTCAT-3′ (SEQ ID NO:42) and 5′-CCAGACACCTGATGAGAAT-3′ (SEQ ID NO:43) (see Huang et al., 24(23) J. CELL. MOL. MED. 13949-13960 (2020)). An IGF2BP3 siRNA can also comprise 5′-GCAGGAATTGACGCTGTATAA-3′ (SEQ ID NO:44), 5′-CGGTGAATGAACTTCAGAATT-3′ (SEQ ID NO:45) or 5′-TGTTGTAGTCTCACAGTATAA-3′ (SEQ ID NO:46) (see Ge et al., 15 CELL DEATH DIS. 192 (2024)). The mRNA sequence of IGF2BP3 (NCBI Reference Sequence: NM_006547.3) is shown in SEQ ID NO:47. The coding sequence is shown in nucleotides 382-2121 of SEQ ID NO:47. The amino acid sequence of IGF2BP3 is shown in SEQ ID NO:48.

The sequence listing for SEQ ID NOS:3, 6-8, 10-11, 14-15, 18-23, 26-30, 35-36 and 39-40 all contain thymines where indicated as uracil in the above text. The software used for generating the sequences would only accept thymine as an input nucleotide. It is understood that one of ordinary skill in the art would understand the explicit text written above or the sequences in the sequence listing, or a combination thereof, may be used as siRNA.

### IV. CHIMERIC ANTIGEN RECEPTORS (CARS)

In one aspect, the present invention provides exosomes isolated from CAR T-cells. In general, a CAR comprises at least one antigen binding domain, at least one transmembrane domain, and at least one intracellular domain. More specifically, a chimeric antigen receptor (CAR) is an artificially constructed hybrid protein or polypeptide containing the antigen binding domains of an antibody (e.g., single chain variable fragment (ScFv)) linked to T-cell signaling domains via the transmembrane domain. Characteristics of CARs include their ability to redirect T-cell specificity and reactivity toward a selected target in a non-MHC-restricted manner, and exploiting the antigen-binding properties of monoclonal antibodies. The non-MHC-restricted antigen recognition gives T cells expressing CARs the ability to recognize antigen independent of antigen processing, thus bypassing a major mechanism of tumor escape. Moreover, when expressed in T-cells, CARs advantageously do not dimerize with endogenous T cell receptor (TCR) alpha and beta chains.

In certain embodiments, the intracellular T cell signaling domains of the CARs can include, for example, a T cell receptor signaling domain, a T cell costimulatory signaling domain, or both. The T cell receptor signaling domain refers to a portion of the CAR comprising the intracellular domain of a T cell receptor. The costimulatory signaling domain refers to a portion of the CAR comprising the intracellular domain of a costimulatory molecule, which is a cell surface molecule other than an antigen receptor or their ligands that are required for an efficient response of lymphocytes to antigen.

### A. Extracellular Domain

In one embodiment, a CAR comprises a target-specific binding element otherwise referred to as an antigen binding domain or moiety. The choice of domain depends upon the type and number of ligands that define the surface of a target cell. For example, the antigen binding domain may be chosen to recognize a ligand that acts as a cell surface marker on target cells associated with a particular disease state.

In one embodiment, the CAR can be engineered to target a tumor antigen of interest by way of engineering a desired antigen binding domain that specifically binds to an antigen on a tumor cell. Tumor antigens are proteins that are produced by tumor cells that elicit an immune response, particularly T-cell mediated immune responses. The selection of the antigen binding domain will depend on the particular type of cancer to be treated. Tumor antigens include, for example, an immune checkpoint marker (e. g., B7-H3), a glioma-associated antigen, carcinoembryonic antigen (CEA), 3-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1 a, p53, prostein, PSMA, Her2/neu, survivin and telomerase, prostate-carcinoma tumor antigen-I (PCTA-1), MAGE, ELF2M, neutrophil elastase, ephrinB2, CD20, CD22, RORI, insulin growth factor (IGF)-I, IGF-11, IGF-I receptor and CD19. The tumor antigens disclosed herein are merely included by way of example. The list is not intended to be exclusive and further examples will be readily apparent to those of skill in the art.

The type of tumor antigen may also be a tumor-specific antigen (TSA) or a tumor-associated antigen (TAA). A TSA is unique to tumor cells and does not occur on other cells in the body. A TAA is not unique to a tumor cell and instead is also expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during fetal development when the immune system is immature and unable to respond or they may be antigens that are normally present at extremely low levels on normal cells but which are expressed at much higher levels on tumor cells.

Non-limiting examples of TSAs or TAAs include the following: Differentiation antigens such as MART-I/MelanA (MART-I), gplOO (Pmel I7), tyrosinase, TRP-I, TRP-2 and tumor-specific multi-lineage antigens such as MAGE-I, MAGE-3, BAGE, GAGE-I, GAGE-2, pI5; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-I 80, MAGE-4, MAGE-5, MAGE-6, RAGE, NY-ESO, pI85erbB2, pI80erbB-3, c-met, nm-23HI, PSA, TAG-72, CA I9-9, CA 72-4, CAM 17.I, NuMa, K-ras, beta-Catenin, CDK4, Mum-I, p 15, p 16, 43-9F, 5T4, 79ITgp72, alpha-fetoprotein, beta-HCG, BCA225, BTAA, CA 125, CA I5-3\CA 27.29\BCAA, CA 195, CA 242, CA-50, CAM43, CD68\PI, C0-029, FGF-5, G250, Ga733\EpCAM, HTgp-I75, M344, MA-50, MG7-Ag, MOVI8, NB/70K, NY-CO-I, RCASI, SDCCAGI6, TA-90\Mac-2 binding protein\cyclophilin C-associated protein, TAAL6, TAG72, TLP, and TPS.

In one embodiment, the antigen binding domain portion of the CAR targets an antigen that includes but is not limited to B7-H3, CDI9, CD20, CD22, RORI, CD33, CD38, CDI23, CDI38, BCMA, c-Met, PSMA, Glycolipid F77, EGFRvIII, GD-2, FGFR4, TSLPR, NY-ESO-I TCR, MAGE A3 TCR, and the like.

### B. Transmembrane Domain

With respect to the transmembrane domain, a CAR comprises one or more transmembrane domains fused to the extracellular antigen binding domain of the CAR. The transmembrane domain may be derived either from a natural or from a synthetic source.

Where the source is natural, the domain may be derived from any membrane-bound or transmembrane protein.

Transmembrane regions of particular use in the CARs described herein may be derived from (i.e., comprise at least the transmembrane region(s) of), but are not limited to, the alpha, beta or zeta chain of the T-cell receptor, CD2S, CD3 epsilon, CD45, CD4, CDS, CD9, CD16, CD22, mesothelin, CD33, CD37, CD64, CDSO, CDS3, CDS6, CD134, CD137, CD154, TNFRSF16, or TNFRSF19.

Alternatively the transmembrane domain may be synthetic, in which case it will comprise predominantly hydrophobic residues such as leucine and valine. Preferably a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the cytoplasmic signaling domain of the CAR. A glycine-serine doublet provides a particularly suitable linker.

In one embodiment, the transmembrane domain that naturally is associated with one of the domains in the CAR is used in addition to the transmembrane domains described herein.

In some instances, the transmembrane domain can be selected or by amino acid substitution to avoid binding of such domains to the transmembrane domains of the same or different surface membrane proteins to minimize interactions with other members of the receptor complex.

### C. Spacer (Hinge, H) Domain

In certain CAR embodiments, a spacer domain can be arranged between the extracellular domain and the transmembrane domain, or between the intracellular domain and the transmembrane domain. The spacer domain means any oligopeptide or polypeptide that serves to link the transmembrane domain with the extracellular domain and/or the transmembrane domain with the intracellular domain. The spacer domain comprises up to 300 amino acids, preferably 10 to 100 amino acids, and most preferably 25 to 50 amino acids.

In several embodiments, the linker can include a spacer element which, when present, increases the size of the linker such that the distance between the effector molecule or the detectable marker and the antibody or antigen binding fragment is increased. Exemplary spacers are known to the person of ordinary skill, and include those listed in U.S. Pat. Nos. 7,964,566; 7,495,295; 6,554,569; 6,323,315; 6,239,104; 6,034,065; 5,750,555; 5,665,560; 5,663,149; 5,635,453; 5,599,902; 5,554,725; 5,530,097; 5,521,254; 5,504,191; 5,410,024; 5,135,036; 5,076,973, 4,956,955; 4,975,744; 4,579,275; 4,516,444; and 4,45,414, as well as U.S. Pat. Pub. Nos. 20110212055 and 20110070245, each of which is incorporated by reference herein in its entirety.

In particular embodiments, the spacer domain comprises a sequence that promotes binding of a CAR with an antigen and enhances signaling into a cell. Examples of an amino acid that is expected to promote the binding include cysteine, a charged amino acid, and serine and threonine in a potential glycosylation site, and these amino acids can be used as an amino acid constituting the spacer domain.

In other embodiments, a spacer domain can comprise elements of Immunoglobulin (lg) constant domains including sequences that link immunoglobulin domains that comprise an immunoglobulin protein.

In further CAR embodiments, a signal peptide sequence can be linked to the N-terminus. The signal peptide sequence exists at the N-terminus of many secretory proteins and membrane proteins, and has a length of about 15 to about 30 amino acids. Because many of the protein molecules mentioned above as the intracellular domain have signal peptide sequences, the signal peptides can be used as a signal peptide for the CAR.

### D. Intracellular Domain

The cytoplasmic domain or otherwise the intracellular signaling domain of the CAR is responsible for activation of at least one of the normal effector functions of the immune cell in which the CAR has been placed. The term “effector function” refers to a specialized function of a cell. Effector function of a T cell, for example, may be cytolytic activity or helper activity including the secretion of cytokines. Thus the term “intracellular signaling domain” refers to the portion of a protein which transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

Examples of intracellular signaling domains for use in the CAR include the cytoplasmic sequences of the T cell receptor (TCR) and co-receptors that act in concert to initiate signal transduction following antigen receptor engagement, as well as any derivative or variant of these sequences and any synthetic sequence that has the same functional capability.

It is known that signals generated through the TCR alone are insufficient for full activation of the T cell and that a secondary or co-stimulatory signal is also required. Thus, T cell activation can be said to be mediated by two distinct classes of cytoplasmic signaling sequence: those that initiate antigen-dependent primary activation through the TCR (primary cytoplasmic signaling sequences) and those that act in an antigen-independent manner to provide a secondary or co-stimulatory signal (secondary cytoplasmic signaling sequences).

Primary cytoplasmic signaling sequences regulate primary activation of the TCR complex either in a stimulatory way, or in an inhibitory way. Primary cytoplasmic signaling sequences that act in a stimulatory manner may contain signaling motifs which are known as immunoreceptor tyrosine-based activation motifs or ITAMs.

### E. Additional Description of CARs

Also expressly included within the scope of the invention are functional portions of CARs. The term “functional portion” when used in reference to a CAR refers to any part or fragment of one or more of a CAR, which part or fragment retains the biological activity of the CAR of which it is a part (the parent CAR). Functional portions encompass, for example, those parts of a CAR that retain the ability to recognize target cells, or detect, treat, or prevent a disease, to a similar extent, the same extent, or to a higher extent, as the parent CAR. In reference to the parent CAR, the functional portion can comprise, for instance, about 10%, 25%, 30%, 50%, 68%, 80%, 90%, 95%, or more, of the parent CAR

The functional portion can comprise additional amino acids at the amino or carboxy terminus of the portion, or at both termini, which additional amino acids are not found in the amino acid sequence of the parent CAR Desirably, the additional amino acids do not interfere with the biological function of the functional portion, e.g., recognize target cells, detect cancer, treat or prevent cancer, etc. More desirably, the additional amino acids enhance the biological activity, as compared to the biological activity of the parent CAR.

Included in the scope of the disclosure are functional variants of the CARs disclosed herein. The term “functional variant” as used herein refers to a CAR, polypeptide, or protein having substantial or significant sequence identity or similarity to a parent CAR, which functional variant retains the biological activity of the CAR of which it is a variant. Functional variants encompass, for example, those variants of the CAR described herein (the parent CAR) that retain the ability to recognize target cells to a similar extent, the same extent, or to a higher extent, as the parent CAR In reference to the parent CAR, the functional variant can, for instance, be at least about 30%, 50%, 75%, 80%, 90%, 98% or more identical in amino acid sequence to the parent CAR.

A functional variant can, for example, comprise the amino acid sequence of the parent CAR with at least one conservative amino acid substitution. Alternatively, or additionally, the functional variants can comprise the amino acid sequence of the parent CAR with at least one non-conservative amino acid substitution. In this case, it is preferable for the non-conservative amino acid substitution to not interfere with or inhibit the biological activity of the functional variant. The non-conservative amino acid substitution may enhance the biological activity of the functional variant, such that the biological activity of the functional variant is increased as compared to the parent CAR.

Amino acid substitutions of the CARs are preferably conservative ammo acid substitutions. Conservative amino acid substitutions are known in the art, and include amino acid substitutions in which one amino acid having certain physical and/or chemical properties is exchanged for another amino acid that has the same or similar chemical or physical properties. For instance, the conservative amino acid substitution can be an acidic/negatively charged polar amino acid substituted for another acidic/negatively charged polar amino acid (e.g., Asp or Glu), an amino acid with a nonpolar side chain substituted for another amino acid with a nonpolar side chain (e.g., Ala, Gly, Val, He, Leu, Met, Phe, Pro, Trp, Cys, Val, etc.), a basic/positively charged polar amino acid substituted for another basic/positively charged polar amino acid (e.g., Lys, His, Arg, etc.), an uncharged amino acid with a polar side chain substituted for another uncharged amino acid with a polar side chain (e.g., Asn, Gin, Ser, Thr, Tyr, etc.), an amino acid with a beta-branched side-chain substituted for another amino acid with a beta-branched side-chain (e.g., He, Thr, and Val), an amino acid with an aromatic side-chain substituted for another amino acid with an aromatic side chain (e.g., His, Phe, Trp, and Tyr), etc.

The CARs (including functional portions and functional variants) can be of any length, i.e., can comprise any number of amino acids, provided that the CARs (or functional portions or functional variants thereof) retain their biological activity, e.g., the ability to specifically bind to antigen, detect diseased cells in a mammal, or treat or prevent disease in a mammal, etc. For example, the CAR can be about 50 to about 5000 amino acids long, such as 50, 70, 75, 100, 125, 150, 175, 200, 300, 400, 500, 600, 700, 800, 900, 1000 or more amino acids in length.

The CARs (including functional portions and functional variants of the invention) can comprise synthetic amino acids in place of one or more naturally-occurring amino acids. Such synthetic amino acids are known in the art.

The CARs (including functional portions and functional variants) can be glycosylated, amidated, carboxylated, phosphorylated, esterified, N-acylated, cyclized via, e.g., a disulfide bridge, or converted into an acid addition salt and/or optionally dimerized or polymerized, or conjugated.

The CARs (including functional portions and functional variants thereof) can be obtained by methods known in the art. The CARs may be made by any suitable method of making polypeptides or proteins. Suitable methods of de nova synthesizing polypeptides and proteins are described in references, such as Chan et al., Fmoc Solid Phase Peptide Synthesis, Oxford University Press, Oxford, United Kingdom, 2000; Peptide and Protein Drug Analysis, ed. Reid, R., Marcel Dekker, Inc., 2000; Epitope Mapping, ed. Westwood et al., Oxford University Press, Oxford, United Kingdom, 2001; and U.S. Pat. No. 5,449,752. Also, polypeptides and proteins can be recombinantly produced using the nucleic acids described herein using standard recombinant methods. See, for instance, Sambrook et al., Molecular Cloning: A Laboratory Manual, 3rd ed., Cold Spring Harbor Press, Cold Spring Harbor, NY 2001; and Ausubel et al., Current Protocols in Molecular Biology, Greene Publishing Associates and John Wiley & Sons, NY, 1994. Further, some of the CARs (including functional portions and functional variants thereof) can be isolated and/or purified from a source, such as a plant, a bacterium, an insect, a mammal, e.g., a rat, a human, etc. Methods of isolation and purification are well-known in the art. Alternatively, the CARs described herein (including functional portions and functional variants thereof) can be commercially synthesized by companies. In this respect, the CARs can be synthetic, recombinant, isolated, and/or purified.

### V. THERAPEUTIC METHODS

Provided are methods of treating a disease or disorder in a subject, the method comprising administration to the subject a composition described herein. In one embodiment, the mature exosomes are administered to a subject. The subject may be the same subject from which the body fluid was collected. The method of administering the exosomes includes, but is not limited to: intravenous, intra-arterial, intrathecal, intraventricular, subcutaneous, subdermal, oral, rectal, intraperitoneal, transdermal, intraosseous injection, intraosseous infusion, or a combination thereof. In one embodiment the mature exosomes are administered in vivo.

Some embodiments provided herein describe methods of treating cancer, wherein the method comprises treating a patient with any one of the compositions described herein. Some embodiments provided herein describe methods of treating cancer, wherein the method comprises treating a patient with extracellular vesicles containing one of the siRNAs described herein (e.g., TTC, EpCAM and/or PCA, as well as GPX4 and/or ACSL4). Some embodiments provided herein describe methods of treating cancer, wherein the method comprises treating a patient with cancer cell EVs obtained from the patient, wherein the cancer cell EVs obtained from the patient are contacted with or contain one of the compositions described herein.

In some embodiments, the methods for treating cancer provided herein reduce the tumor size (e.g., volume or diameter) in a subject by at least about 5%, 10%, 15%, 20%, 25%, 30%, 35%, 40%, 45%, 50%, 55%, 60%, 65%, 80%, 85%, 90%, 95%, 99%, or 100%, relative to tumor size (e.g., volume or diameter) prior to administration of a composition as assessed by methods well known in the art, e.g., CT Scan, MRI, DCE-MRI, or PET Scan. In particular embodiments, the methods for treating cancer provided herein reduce the tumor volume or tumor size (e.g., diameter) in a subject by an amount in the range of about 5% to 20%, 10% to 20%, 10% to 30%, 15% to 40%, 15% to 50%, 20% to 30%, 20% to 40%, 20% to 50%, 30% to 60%, 30% to 70%, 30% to 80%, 30% to 90%, 30% to 95%, 30% to 99%, 30% to 100%, or any range in between, relative to tumor size (e.g., diameter) in a subject prior to administration of a composition as assessed by methods well known in the art, e.g., CT Scan, MRI, DCE-MRI, or PET Scan.

In some embodiments, the present invention provides compositions and methods for reducing the amount of a target nucleic acid or tumor progression associated with the target nucleic acid in an extracellular vesicle (e.g., an exosome) derived from a cancer cell. Thus, a composition of the present invention can have anti-tumor activity. In some embodiments, a pharmaceutical composition is administered to an animal having at least one cancer cell. In some embodiments, a pharmaceutical composition is administered to an animal having at least one symptom associated with cancer. In some embodiments, such administration results in amelioration of at least one symptom. In some embodiments, the administration of a composition delays the onset of cancer. In some embodiments, the administration of a composition slows the proliferation of cancer cells. In some embodiments, the administration of a composition slows the proliferation of tumor cells. In some embodiments, the administration of a composition prevents the growth of cancer. In some embodiments, the administration of a composition prevents the formation of tumors. In some embodiments, the administration of a composition causes tumor mass to decrease.

In some embodiments, the methods for treating cancer provided herein inhibit, reduce, diminish, arrest, or stabilize a tumor associated with the cancer. In specific embodiments, the methods for treating cancer provided herein cause the regression of a tumor, tumor blood flow, tumor metabolism, or peritumor edema, and/or one or more symptoms associated with the cancer. In other embodiments, the methods for treating cancer provided herein maintain the size of the tumor so that it does not increase, or so that it increases by less than the increase of a tumor after administration of a standard therapy as measured by conventional methods available to one of skill in the art, such as digital rectal exam, ultrasound (e.g., transrectal ultrasound), CT Scan, MRI, dynamic contrast-enhanced MRI, or PET Scan. In specific embodiments, the methods for treating cancer provided herein decrease tumor size. In some embodiments, the methods for treating cancer provided herein reduce the formation of a tumor. In some embodiments, the methods for treating cancer provided herein eradicate, remove, or control primary, regional and/or metastatic tumors associated with the cancer. In some embodiments, the methods for treating cancer provided herein decrease the number or size of metastases associated with the cancer.

In some embodiments, the compositions and methods described herein treat cancers such as prostate, lung, breast, brain, prostate, spleen, pancreatic, cervical, ovarian, head and neck, esophageal, liver, skin, kidney, leukemia, bone, testicular, colon, or bladder cancer. In some embodiments, the cancer is pancreatic cancer, colon cancer, breast cancer, T-cell leukemias, or lymphomas. In some embodiments, the cancer is leukemia, lymphoma, or multiple myeloma.

Solid tumor cancers that can be treated by the methods provided herein include, but are not limited to, sarcomas, carcinomas, and lymphomas. In specific embodiments, cancers that can be treated in accordance with the methods described include, but are not limited to, cancer of the breast, liver, neuroblastoma, head, neck, eye, mouth, throat, esophagus, esophagus, chest, bone, lung, kidney, colon, rectum or other gastrointestinal tract organs, stomach, spleen, skeletal muscle, subcutaneous tissue, prostate, breast, ovaries, testicles or other reproductive organs, skin, thyroid, blood, lymph nodes, kidney, liver, pancreas, and brain or central nervous system. In some embodiments, the solid tumors that can be treated by the methods provided herein include, but are not limited to, sarcomas, carcinomas, and lymphomas.

Also, provided herein are combination therapies for the treatment of cancer which involve the administration of a composition in combination with one or more additional therapies to a subject in need thereof. In a specific embodiment, presented herein are combination therapies for the treatment of cancer which involve the administration of an effective amount of a composition in combination with an effective amount of another therapy to a subject in need thereof.

Thus, in one aspect, the present invention provides a prostate cancer therapy or therapeutic intervention practically applied/administered with a composition of the present invention. In particular embodiments, therapeutic intervention comprises prostatectomy, radiation therapy, cryotherapy (also referred to as cryosurgery or cryoablation), hormone therapy, chemotherapy, immunotherapy and combinations thereof.

Prostatectomy includes radical prostatectomy (open (radical retropubic prostatectomy or radical perineal prostatectomy) or lateral (laparoscopic radical prostatectomy including robotic-assisted), and transurethral resection of the prostate (TURP).

Radiation therapy includes external beam radiation (three-dimensional conformal radiation therapy (3D-CRT), intensity modulated radiation therapy (IMRT), stereotactic body radiation therapy (SBRT), proton beam radiation therapy) and brachytherapy (internal radiation) (permanent (low dose rate or LDR) brachytherapy or temporary (high dose rate or HDR) brachytherapy).

Hormone therapy (androgen suppression therapy) includes orchiectomy (surgical castration), luteinizing hormone-release hormone (LHRH) agonists (e.g., leuprolide, goserelin, triptorelin, histrelin), LHRH antagonists (e.g., degarelix), treatment to lower androgen levels from the adrenal glands (e.g., abiraterone, ketoconazole), anti-androgens (e.g., flutamide, bicalutamide, nilutamide, enzalutamide, apalutamide), and estrogens.

Chemotherapy includes treatment with compounds including, but not limited to, docetaxel, cabazitaxel, mitoxantrone, and estramustine.

Immunotherapy includes, but is not limited to, a cancer vaccine (e.g., sipuleucel-T), as well as immune checkpoint inhibitors (e.g., PD-1 inhibitors including pembrolizumab). Illustrative immune checkpoint inhibitors include Tremelimumab (CTLA-4 blocking antibody), anti-OX40, PD-L1 monoclonal Antibody (Anti-B7-H1; MEDI4736), MK-3475 (PD-1 blocker), Nivolumab (anti-PD1 antibody), CT-011 (anti-PD1 antibody), BY55 monoclonal antibody, AMP224 (anti-PDL1 antibody), BMS-936559 (anti-PDL1 antibody), MPLDL3280A (anti-PDL1 antibody), MSB0010718C (anti-PDL1 antibody) and Yervoy/ipilimumab (anti-CTLA-4 checkpoint inhibitor).

A prostate therapeutic intervention can comprise a targeted therapy including poly(ADP)-ribose polymerase (PARP) inhibitor (e.g., niraparib (zejula), olaparib (lynparza), talazoparib (talzenna) and rucaparib (rubraca)).

For example, Rucaparib (Rubraca) can be used to treat metastatic, castration-resistant prostate cancer (mCRPC) that has grown after treatment with taxane chemotherapy (such as docetaxel or cabazitaxel) and hormone therapy, and when the cancer cells have a change in one of the BRCA genes.

In some embodiments, Olaparib (Lynparza) can be used to treat mCRPC, either: (1) along with the hormone therapy drug abiraterone (and a steroid drug such as prednisone), if the cancer cells have changes in one of the BRCA genes; or (2) by itself, if the cancer has grown after getting a hormone therapy drug such as enzalutamide or abiraterone, and the cancer cells have a change in one of the BRCA genes (or other DNA repair genes known as homologous recombinant repair (HRR) genes). HRR genes include: BRCA1, BRCA2, ATM, ATR, BARD1, BRIP1, CDK12, CHEK1, CHEK2, FANCA, FANCL, MLH1, MRE11A, NBN, PALB2, RAD51B, RAD51C, RAD51D, and RAD54L.

Talazoparib (Talzenna) can be used along with the hormone therapy drug enzalutamide to treat mCRPC, if the cancer cells have a change (mutation) in a homologous recombination repair (HRR) gene.

Niraparib plus abiraterone (Akeega) can be used (along with the steroid drug prednisone) to treat mCRPC if the cancer cells have a change in one of the BRCA genes.

Other therapeutic interventions for prostate cancer include an androgen receptor (AR)-targeted therapy (e.g., enzalutamide, ARN-509, ODM-201, EPI-001, hydrazinobenzoylcurcumin (HBC), aberaterone, geleterone, and seviteronel), an antimicrotubule agent, an alkylating agent and an anthracenedione.

In particular embodiments, a therapeutic intervention for prostate cancer can include the administration of drugs including, but not limited to, Abiraterone Acetate, Apalutamide, Bicalutamide, Cabazitaxel, Casodex (Bicalutamide), Darolutamide, Degarelix, Docetaxel, Eligard (Leuprolide Acetate), Enzalutamide, Erleada (Apalutamide), Firmagon (Degarelix), Flutamide, Goserelin Acetate, Jevtana (Cabazitaxel), Leuprolide Acetate, Lupron (Leuprolide Acetate), Lupron Depot (Leuprolide Acetate), Lynparza (Olaparib), Mitoxantrone Hydrochloride, Nilandron (Nilutamide), Nilutamide, Nubeqa (Darolutamide), Olaparib, Provenge (Sipuleucel-T), Radium 223 Dichloride, Rubraca (Rucaparib Camsylate), Rucaparib Camsylate, Sipuleucel-T, Taxotere (Docetaxel), Xofigo (Radium 223 Dichloride), Xtandi (Enzalutamide), Zoladex (Goserelin Acetate), Zytiga (Abiraterone Acetate).

In some embodiments, a therapeutic intervention for medulloblastoma can include, but are not limited to, pemetrexed, gemcitabine (including combination with pemetrexed), vismodegib, sonidegib, panobinostat, romidepsin, belinostat, CI-994 (tacedinaline), silmitasertib, palbociclib, sapanisertib, as well as a combination of palbociclib and sapanisertib (e.g., delivered in nanoparticles or extracellular vesicles such as exosomes).

Treatment can include maximal safe surgical resection, radiotherapy and chemotherapy (e.g., cisplatin, cyclophosphamide, vincristine, lomustine, in various dosing regimens; standard dosing is typically 9 cycles, high dose is typically 4 cycles). Combination treatment can be used including, but not limited to, pemetrexed and gemcitabine. Surgery may be needed to treat hydrocephalus (fluid build-up in the skull) and to remove the tumor. Treatment can further include (alone or in combination) endoscopic third ventirculostomy (ETV) or ventriculo-peritoneal shunt (VP shunt).

Drugs already FDA-approved for brain cancers include Afinitor (Everolimus), Afinitor Disperz (Everolimus), Alymsys (Bevacizumab), Avastin (Bevacizumab), Belzutifan, Bevacizumab, BiCNU (Carmustine), Carmustine, Carmustine Implant, Danyelza (Naxitamab-gqgk), Dabrafenib Mesylate, Eflornithine Hydrochloride, Everolimus, Gliadel Wafer (Carmustine Implant), Iwilfin (Eflornithine Hydrochloride), Lomustine, Mekinist (Trametinib Dimethyl Sulfoxide), Mvasi (Bevacizumab), Ojemda (Tovorafenib), Tafinlar (Dabrafenib Mesylate), Temodar (Temozolomide), Temozolomide, Tovorafenib, Trametinib Dimethyl Sulfoxide, Voranigo (Vorasidenib Citrate), Welireg (Belzutifan) and Zirabev (Bevacizumab), as well as a combination of PCV (procarbazine hydrochloride, lomustine and vincristine sulfate). Such drugs could be used individually or in combination for the treatment of medulloblastoma.

It is understood that the EVs such as exosomes of the present invention may be loaded with other cargo such as the drugs recited above. Exosomes may be loaded with both siRNA and small molecules or a composition can comprise exosomes loaded with siRNA and exosomes loaded with small molecules, antibodies, and the like.

Without further elaboration, it is believed that one skilled in the art, using the preceding description, can utilize the present invention to the fullest extent. The following examples are illustrative only, and not limiting of the remainder of the disclosure in any way whatsoever.

### EXAMPLES

The following examples are put forth so as to provide those of ordinary skill in the art with a complete disclosure and description of how the compounds, compositions, articles, devices, and/or methods described and claimed herein are made and evaluated, and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Example 1: Exosome-Mediated Targeted Drug Delivery to Treat Prostate Cancer

PCa is considered a “cold tumor” with a poor response to conventional immunotherapies such as immune checkpoint inhibitors, vaccines, and CAR-T cells. As further described herein, CAR-T cell-derived exosomes (CAR-Exo) show significant anti-tumor immune responses with low immunotoxicity [7]. Additionally, CAR-Exos can deliver anti-tumor agents to tumor cells that overexpress B7-H3. Hence, anti-B7-H3-CAR-Exos will be loaded with si-TTC3, si-EpCAM and/or PCA3 to specifically target si-TTC3, si-EpCAM and/or PCA3 in tumor tissue, which will inhibit PCa growth and tolerance as well as induce anti-tumor immune responses. The present inventor postulated that combining anti-B7-H3-CAR-Exo and si-TTC3, si-EpCAM and/or PCA3 will provide a powerful, synergistic targeted immunotherapy against PCa that can downregulate tumor growth and enhance specific anti-tumor immune responses.

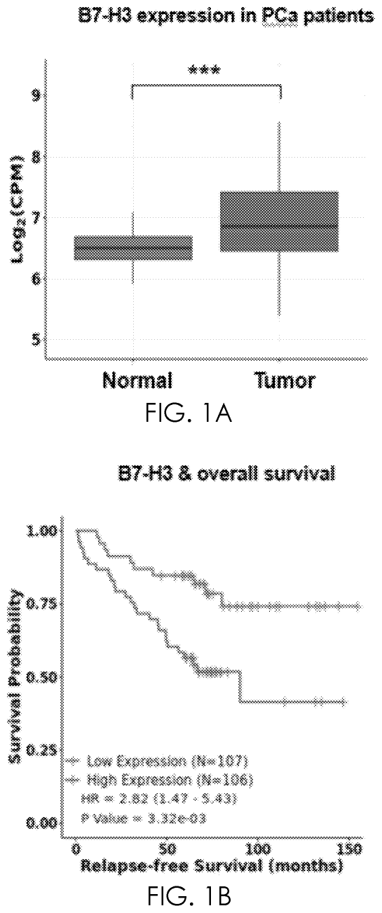

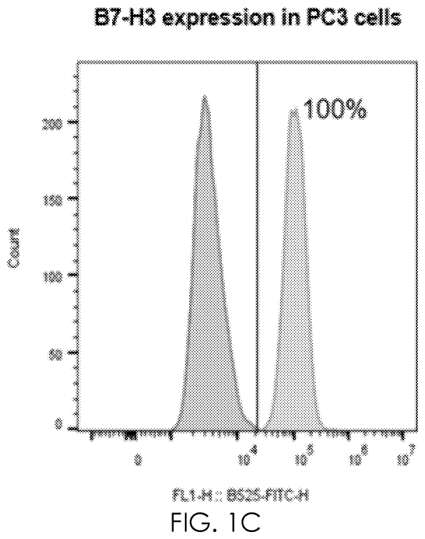

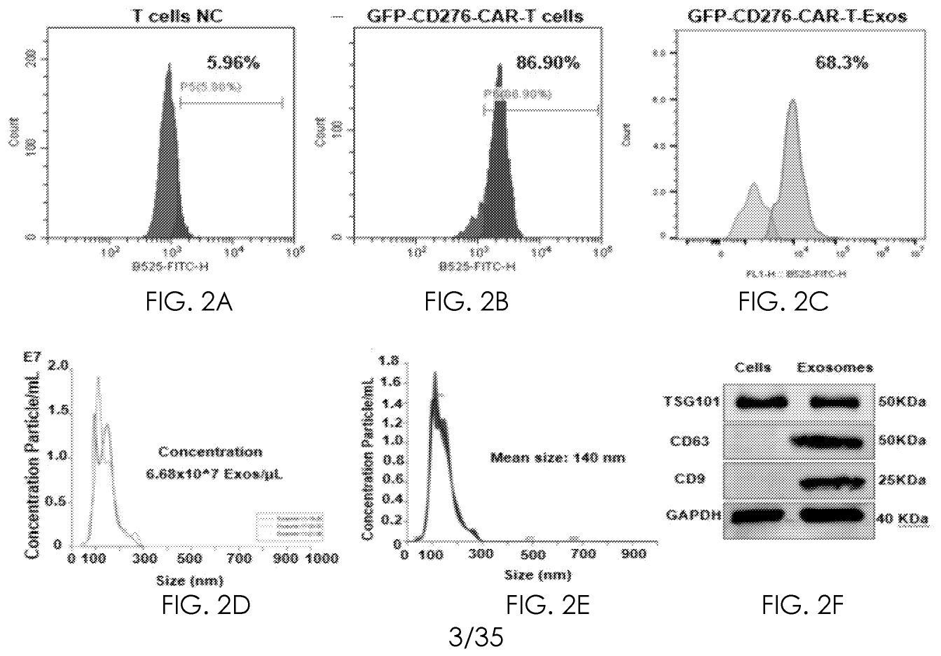

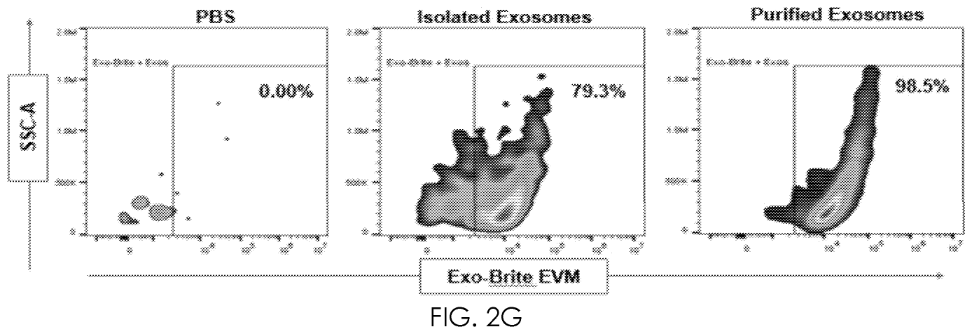

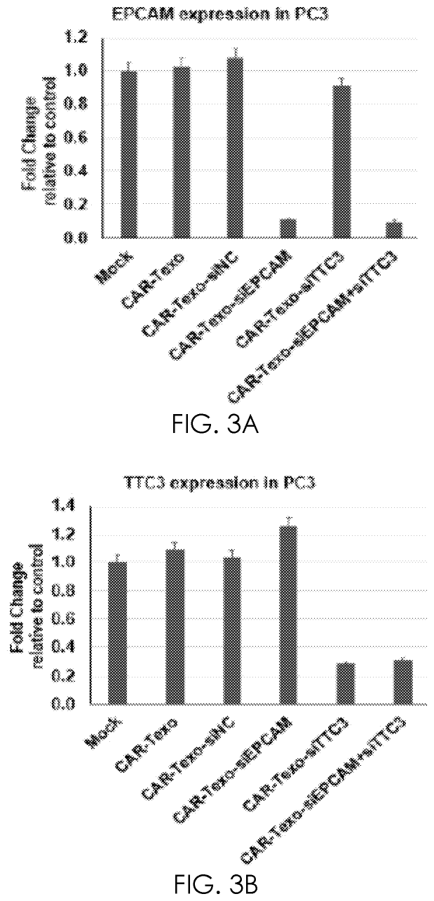

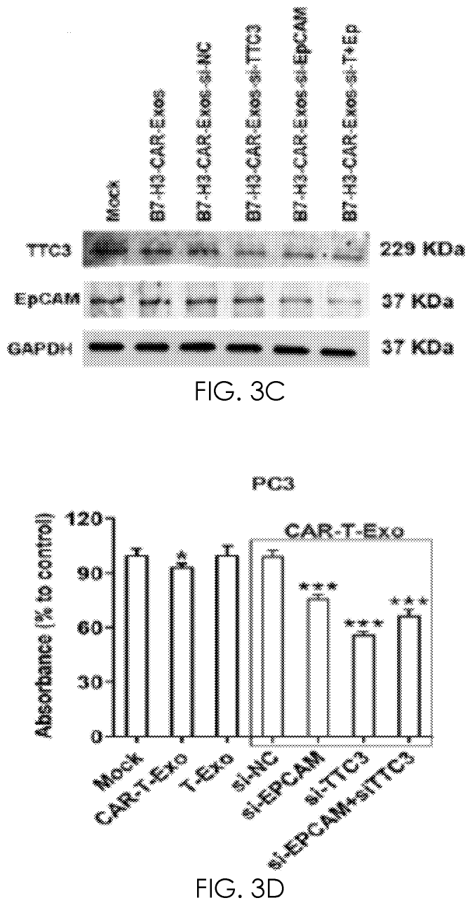

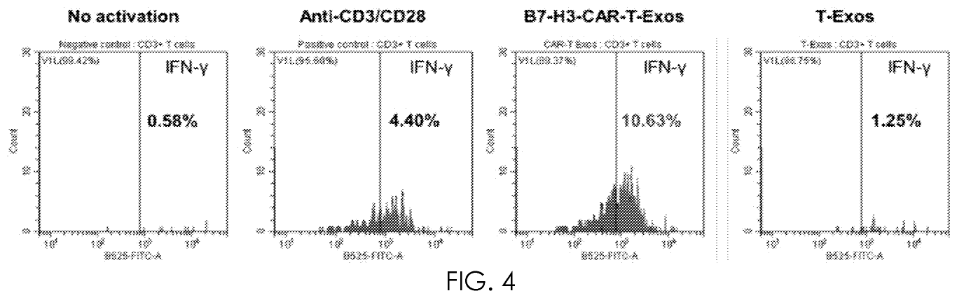

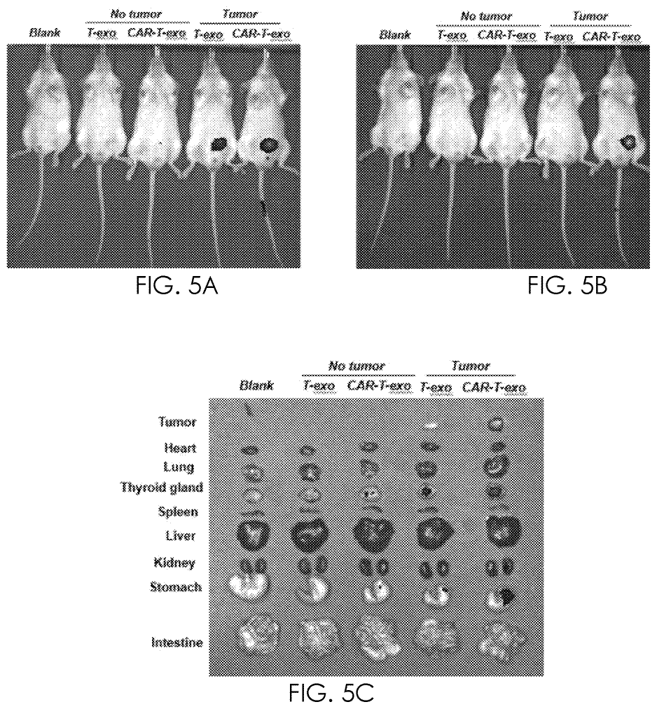

The present inventor has shown that the B7-H3, an immune checkpoint marker, is significantly overexpressed on the prostate cancer cell line PC3 surface and patient samples (FIG. 1). B7-H3 has recently been reported as a promising target in PCa, especially for CAR-T cell therapy, due to its association with metastasis and poor outcomes. Therefore, we developed a novel alternative immunotherapeutic approach targeting B7-H3 as a surface marker in PCa tumors. By generating B7-H3-CAR-T cells (FIG. 2A-2B), we successfully isolated B7-H3 (mbA) CAR-expressing exosomes (FIG. 2C), confirmed by quality and size (FIG. 2D-2G). B7-H3(mbA) CAR-Exos loaded with si-TTC3 and/or si-EpCAM significantly inhibited the expression of target genes (TTC3, EpCAM) in PC3 cells (FIG. 3A-3C). Furthermore, B7-H3(mbA) CAR-T exosomes loaded with si-TTC3 and/or si-EpCAM significantly inhibited tumor cell proliferation (FIG. 3D). Adding B7-H3(mbA) CAR-Exos to naïve T cell cultures significantly increased IFN-7, indicating activation of T cells compared to negative control (FIG. 4). Interestingly, tail vein injection of B7-H3(mbA) CAR-Exos loaded with si-TTC3+Alexa fluor 647 showed promising accumulation of exosomes in tumor tissues (FIG. 5). The present inventor strongly believe that delivering si-TTC3 and si-EpCAM as cargoes within B7-H3-CAR-Exo will further amplify the anti-tumor effects by inhibiting tumor growth and inducing specific anti-tumor immunosurveillance.

The present inventor tested the hypothesis using a humanized mouse model (HuCD34-NCG) bearing xenografts and a spontaneous prostate cancer model (C57BL/6-Tg (TRAMP)8247Ng/J×FVB/NJ) F1/J). These two models were enrolled to investigate the potential therapeutic efficiency of B7-H3 (mbA)-CAR-Exo+si-TTC3 and si-Ep-CAM in PCa.

### Materials and Methods

Construction of the B7-H3-CAR lentiviral vector. Briefly, the pCDCAR1 cloning vector was used to construct the lentivirus vector that sequentially contains a mouse anti-human B7-H3(mbA) single-chain variable fragment sequence (scFv), CD8 hinge and transmembrane domains, and the intracellular signaling domains of 4-1BB and CD3ζ intracellular domain. B7-H3-CAR was connected to GFP by a p2A peptide to generate membrane-bound GFP (mGFP). B7-H3(mbA)-CAR-GFP was inserted into a plasmid pCDCAR1 to produce a lentiviral vector by transducing 293T cells. The particles were aliquoted and stored at −80 °C.

Generating anti-B7-H3(mbA)-GFP-CAR-T cells and harvesting ant-B7-H3(mbA)-CAR-T exosomes. Human CD3⁺ T cells were sorted from PBMCs obtained from healthy donors using negative selection kits from Stemcell. Then, CD3⁺ cells were tested by flow cytometry to confirm purity. CD3⁺ T cells were cultured and expanded in ImmunoCult-XF T cell Expansion Medium from Stemcell supplemented with soluble anti-CD3/anti-CD28 activator and IL-2 [8]. After 3 days of CD3⁺ T cell activation, cells were transduced with B7-H3(mbA)-CAR lentivirus. The expression of B7-H3(mbA)-CAR was assessed by flow cytometry. Then, B7-H3(mbA)-CAR exosomes were harvested from the medium of CAR-T cells using differential ultracentrifugation (SW32Ti Rotor, 5000 rpm for 10 min; 10,000 rpm for 10 min; 25,000 rpm for 120 min) and size exclusion chromatography for purification. The isolated B7-H3(mbA)-CAR exosomes were quantified and characterized using nanoparticle tracking analysis (NTA), transmission electron microscopy (TEM), flow cytometry, and western blotting. B7-H3(mbA)-CAR exosomes were stored at −80 °C for further experiments.

Loading si-TTC3 and si-EpCAM into B7-H3(mbA)-CAR exosomes. si-TTC3, si-EpCAM, or si-NC was loaded into B7-H3(mbA)-CAR exosomes using electroporation with some modifications. Briefly, 2.5 μL of si-RNA was added to 150-200 μg of B7-H3(mbA)-CAR exosomes and incubated at RT for 30 min. Then, an equal volume of electroporation buffer was added to the mixture and subjected to the gene pulser at 125 V and 50 ρF for 10 ms. After that, electroporated exosomes were recovered by incubation in a water bath at 37 °C for 30 min. si-RNA residue and buffer was removed using special chromatography columns. Isolated exosomes were ready for experimental use.

Testing the capability of B7-H3 (mbA)-CAR-T cells to induce tumor toxicity and cell death with/without si-TTC3 and/or si-EpCAM in vitro. Different PCa cell lines were used: (a) 10,000 PCa cells expressing GFP were cultured in a 48-well plate for 24 hours. Then, the effects of 100-150 μg of B7-H3(mbA)-CAR-Exos+si-TTC3/si-EpCAM will be compared with CAR-Exos only and si-NC control. Live cell imaging was performed using intravital microscopy to visualize tumor cell death rates at 12, 24, 36, 48, 60, and 72 hours. (b) The same experiment was used to check GFP⁺ cells after 24, 48, and 72 hours by flow cytometry by gating FITC-positive cells. Differences in GFP levels were represented by cell death, with statistical analysis calculated based on negative controls.

Testing T cell differentiation and proliferation responses to B7-H3(mbA)-CAR-Exos+/−si-TTC3/si-EpCAM. Human primary T cells was isolated from healthy donor PBMCs to test T cell differentiation and proliferation responses to CAR-T-Exos. T cells were stained with CFSE following a published protocol [9]. Next, 100,000 stained T cells was plated into 96-well plates, and 100-150 μg B7-H3(mbA)-CAR-Exos+/−si-TTC3/si-EpCAM will be injected into the cell culture and compared with soluble anti-CD3/CD28 activator as a positive control. Cells were incubated for 5-6 days at 37 °C and 5% CO₂. T cells were collected, washed, and stained with a viability dye, anti-CD4, anti-CD8, and anti-IFN-γ. By flow cytometry, T cell subsets will be analyzed, and T cell differentiation and proliferation in response to B7-H3(mbA)-CAR-Exos+/−si-TTC3/si-EpCAM treatment will be explored. Activation levels were be assessed by determining IFN-7 levels.

Testing activation of immune cells and reduced exhaustion in response to B7-H3(mbA)-CAR-Exos+/−si-TTC3/si-EpCAM. Human PBMCs from healthy donors were used to test immune responses to CAR-Exos in terms of activation and exhaustion markers. 100-200,000 PBMCs will be plated into 48 wells. 100-150 μg of B7-H3(mbA)-CAR-Exos+/−si-TTC3/si-EpCAM was added to the culture. Cells were incubated for 3-5 days, and then cells were stained with viability dye, anti-CD3, anti-CD4, anti-CD8, anti-PD1, anti-TIM3, anti-LAG3, and anti-CTLA4 30 min on ice in the dark and washed twice with FACS buffer. After that, intracellular staining for IFN-γ, IL-17, and FOXP3 was performed according to fixation instructions and the permeabilization kit. Finally, cells were washed twice and tested by flow cytometry.

Investigating the in vivo anti-tumor effects of B7-H3(mbA)-CAR-T-Exos+si-TTC3/si-EpCAM in PCa mouse model. To investigate specific anti-tumor immune responses induced by B7-H3(mbA)-Exos, we used a humanized mouse model (HuCD34-NCG) to evaluate the potential immunotherapeutic effect of our system including systemic immune responses, risks of cytokine release syndrome (CRS), and safe and effective dosing levels of B7-H3(mbA)-CAR-T-Exos. Then, the spontaneous PCa mouse model C57BL/6-Tg (TRAMP)8247Ng/J×FVB/NJ) F1/J were enrolled to investigate potential tumor inhibition effects of B7-H3(mbA)-CAR-T-Exos+si-TTC3/si-EpCAM. Sixty mice were enrolled in each model. Mice were divided into six groups (10 mice/group): normal healthy mice, PCa treated with PBS, PCa mice treated with B7-H3 (mbA)-CAR-T-Exos+si-TTC3/si-EpCAM, PCa mice treated with B7-H3(mbA)-CAR-T-Exos+si-NC, PCa mice treated with control-CAR-T-Exos+siTTC3/si-EpCAM, and PCa mice treated with control-CAR-T-Exos+si-NC. The approach was as follows: (1) 500 μg CAR-Exos loaded with 15 μg si-RNA/injection was injected into mice by tail vein injection twice a week (3-day interval). The therapeutic course will be 6-8 weeks until the tumor size in untreated mice reaches the endpoint (20% of body weight). Then, all mice were sacrificed to collect the organs including the brain, liver, kidney, heart, lymph nodes, and blood. (2) Tumor samples were harvested and tumor size and volume was measured in all groups using a Peira tumor scanner. (3) The tumor samples was divided into different sections: (a) total RNA and protein was collected to check the expression of target genes using qRT-PCR and western blotting; (b) part of tumor samples were frozen for spatial transcriptomic analysis to assess single cell populations and signals activated by CAR-Exo-siRNA therapy using a published protocol [10]; (c) part of frozen tumors will be subjected to immunofluorescence staining to visualize immune cell populations in the tumor microenvironment, allowing us to explore immune responses to the CAR-Exo-siRNA therapy; (d) histopathological studies were performed to score responses to treatment, with tumor proliferation and differentiation markers such as Ki67 and P501S quantified; (e) immune cells were isolated from tumor samples using a CD45⁺ selection kit and then CD8⁺/CD4⁺ selection kits to validate transcriptomics data. (4) Blood samples were collected from all mice, and the plasma levels of pro-inflammatory cytokines associated with CRS [11] like IL-6, soluble IL-6R, IFN-γ, IL-2, soluble IL-2Rα, IL-6, granulocyte-macrophage colony-stimulating factor (GM-CSF), and IL-32 were measured using ELISA.

For the spontaneous mice model (C57BL/6-Tg (TRAMP)8247Ng/J×FVB/NJ) F1/J). Mouse primary T cells were sorted using a CD3⁺ T cell selection kit. These mouse T cells were transduced by mouse B7-H3(mbA)-CAR vector to generate mmu-B7-H3(mbA)-CAR-T cells. These CAR-T cells were used to harvest and purify mmu-B7-H3(mbA)-CAR-T-Exos, which were loaded with mmu-siTTC3 to be prepared and frozen at −80 °C. These mice initiate tumors spontaneously after 12 weeks of age. At 13 weeks of age, an MRI scan was performed to detect tumor formation. Once cancers were diagnosed, the therapeutic protocol for the humanized mouse model was performed as described above. The experiments and further analyses were same as described for the humanized mouse model.

### REFERENCES

1. Li, J., D. A. Siegel, and J. B. King, Stage-specific incidence rates and trends of prostate cancer by age, race, and ethnicity, United States, 2004-2014. Ann Epidemiol, 2018. 28(5): p. 328-330.
2. Siegel, R. L., A. N. Giaquinto, and A. Jemal, Cancer statistics, 2024. CA Cancer J Clin, 2024. 74(1): p. 12-49.
3. Lu-Yao, G. L., et al., An assessment of radical prostatectomy. Time trends, geographic variation, and outcomes. The Prostate Patient Outcomes Research Team. JAMA, 1993. 269(20): p. 2633-6.
4. Etzioni, R., et al., Asymptomatic incidence and duration of prostate cancer. Am J Epidemiol, 1998. 148(8): p. 775-85.
5. Prensner, J. R., et al., Beyond PSA: the next generation of prostate cancer biomarkers. Sci Transl Med, 2012. 4(127): p. 127rv3.
6. Wilt, T. J., et al., Radical prostatectomy versus observation for localized prostate cancer. New England Journal of Medicine, 2012. 367(3): p. 203-213.
7. Fu, W., et al., CAR exosomes derived from effector CAR-T cells have potent antitumour effects and low toxicity. Nature Communications, 2019. 10(1): p. 4355.
8. MacPherson, S., et al., Clinically relevant T cell expansion media activate distinct metabolic programs uncoupled from cellular function. Mol Ther Methods Clin Dev, 2022. 24: p. 380-393.
9. Quah, B. J., H. S. Warren, and C. R. Parish, Monitoring lymphocyte proliferation in vitro and in vivo with the intracellular fluorescent dye carboxyfluorescein diacetate succinimidyl ester. Nat Protoc, 2007. 2(9): p. 2049-56.
10. Fang, Y., et al., Improvements and challenges of tissue preparation for spatial transcriptome analysis of skull base tumors. Heliyon, 2023. 9(3): p. e14133.
11. Wang, Z. and W. Han, Biomarkers of cytokine release syndrome and neurotoxicity related to CAR-T cell therapy. Biomark Res, 2018. 6(1): p. 4.

### Example 2: Develop a Novel Immunotherapeutic Approach Based on Cd276 (B7-H3)-Car-T Cell-Derived Exosomes Comprising SI-GPX4, SI-ACSL4 and/or MIR-211

Some CAR-T cell therapies for solid tumors produce serious adverse events, but CAR-T cell-derived exosomes (CAR-Exo) can effectively induce anti-tumor immune responses with low immunotoxicity. Therefore, we hypothesize that combining CAR-Exo with si-GPX4, si-ACSL4 and/or miR-211 should provide a novel and enhanced immunotherapeutic response in MBs without the associated toxicities. We will test CAR-Exo in a humanized mouse model (HuCD34-NCG) and the SHH mouse model B6; 129T2-Ptch1tm1Bjw/WreyJ.

### Background

Noncoding (nc)RNAs and their related pathways are increasingly recognized as potential therapeutic targets in many cancers¹⁻³. However, little is known about their therapeutic potential in medulloblastoma (MB), a poor-prognosis central nervous system (CNS) tumor that predominantly affects children and always requires aggressive therapy³⁻¹⁰. Effective clinical translation of ncRNAs for CNS tumors involves not only establishing their mechanism of action but also developing authentic models of human disease to test new agents and delivery strategies that circumvent the blood-brain barrier. Our group was the first to identify a vital role for microRNA-211 (miR-211) in melanoma“−” In this Example, we extend the therapeutic opportunity of miR-211-regulated genes, acyl-CoA synthetase long-chain family member 4 (ACSL4) and glutathione peroxidase 4 (GPX4), which we have established as fatty acid and ferroptosis modulators in MB. Our preliminary results now show that miR-211: (i) is significantly downregulated in MBs from patients, MB cell lines, and MB patient-derived xenografts (PDXs) compared with normal cerebellum but their target genes ACSL4 and GPX4 are upregulated; and (ii) tumors growing from MB cells either overexpressing miR-211 or with GPX4 knock-down injected into mouse cerebella are smaller than those derived from parental cells. Therefore, we postulate that ACSL4 and GPX4 are key therapeutic targets in selected MB patients. Based on our preliminary results, we hypothesize that ACSL4 and GPX4 modify fatty acid oxidation, thereby inducing ferroptosis. Thus, the miR-211-ACSL4-GPX4 axis is an excellent candidate pathway to modulate therapeutically in medulloblastoma.

### Develop A Novel Immunotherapeutic Approach Based On CD276 (B7-H3)-CAR-T Cell-Derived Exosomes

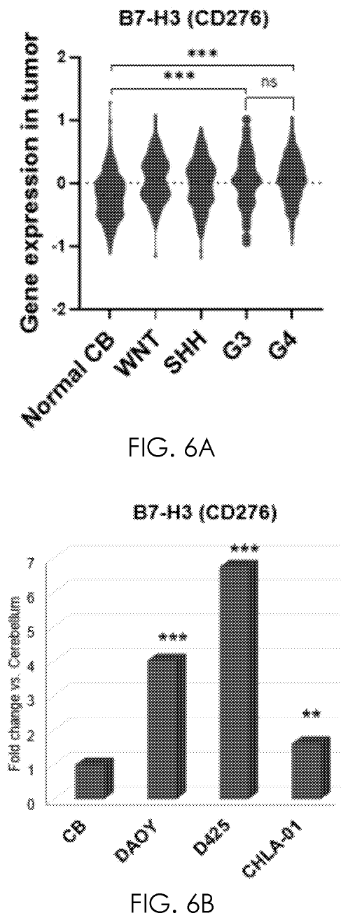

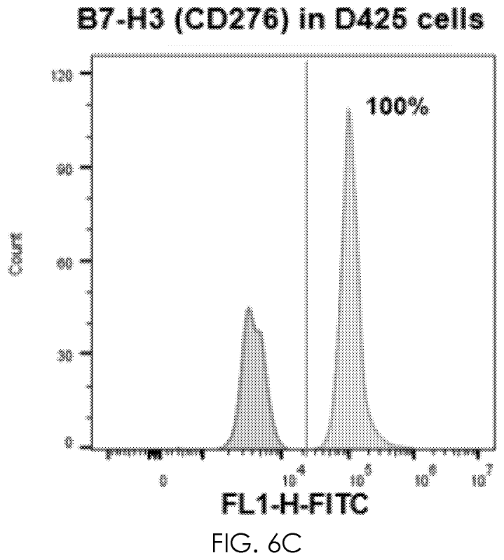

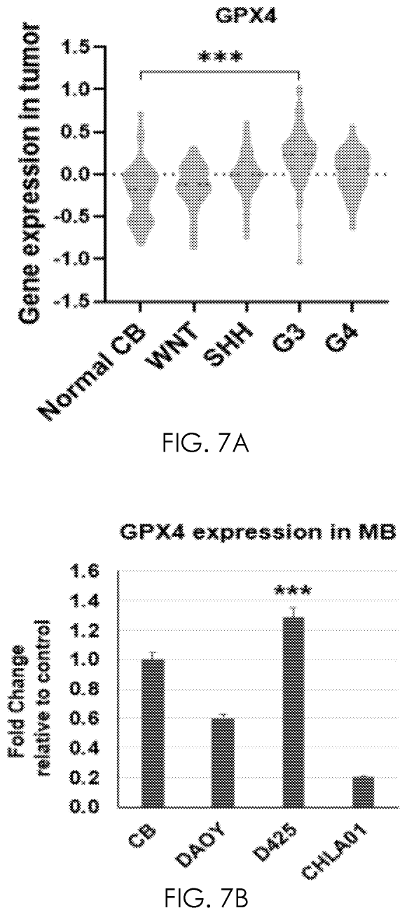

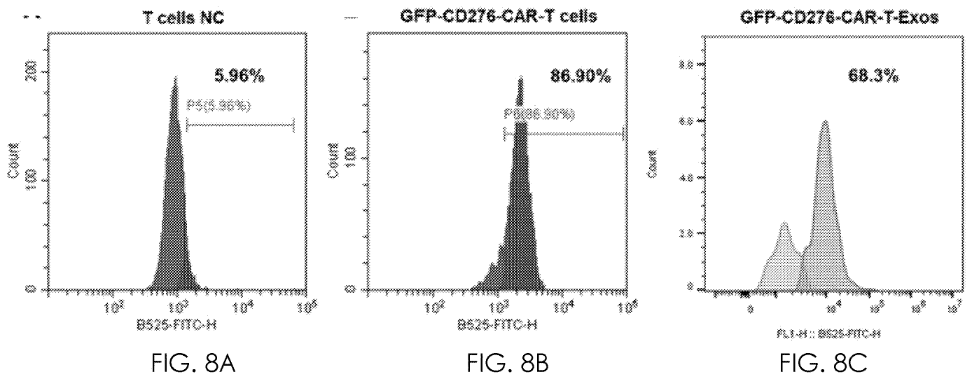

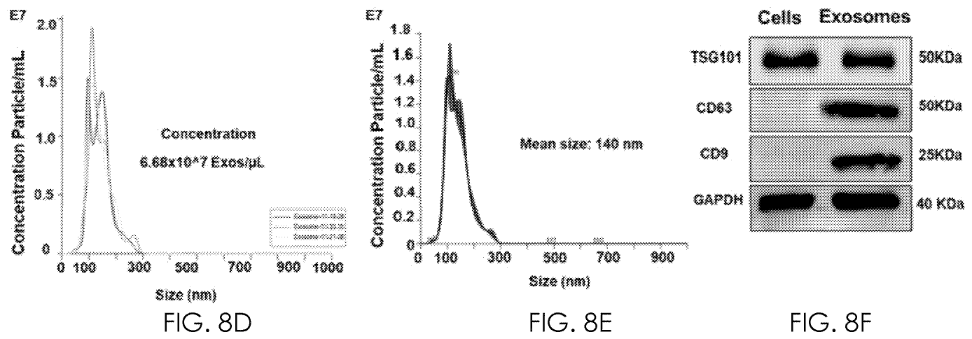

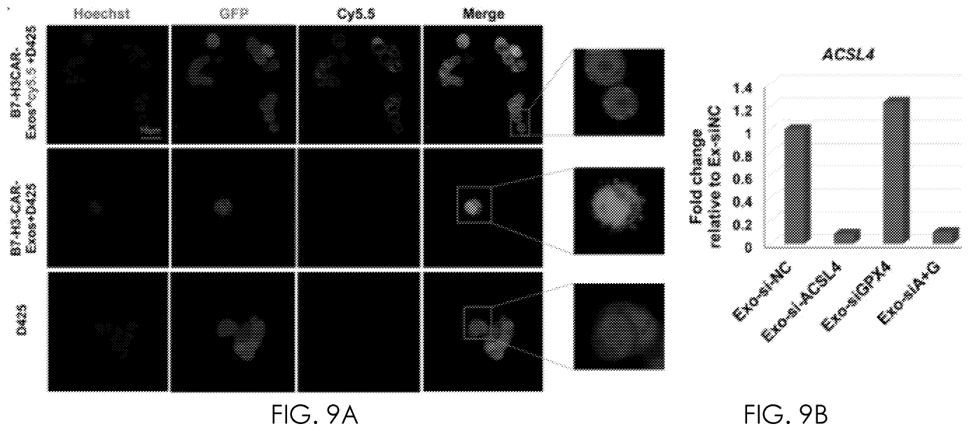

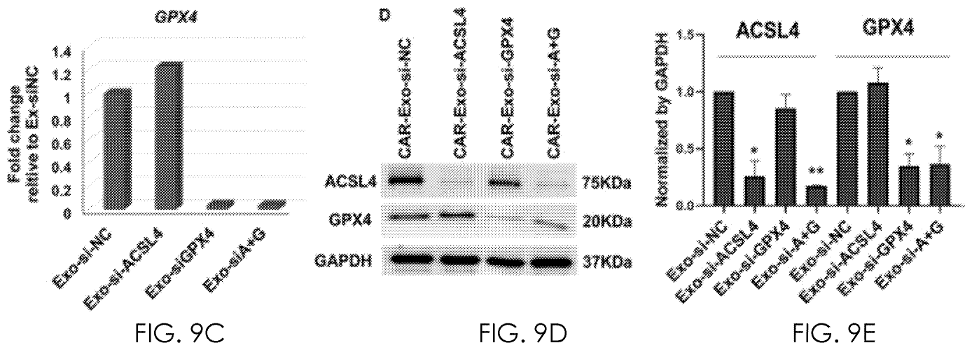

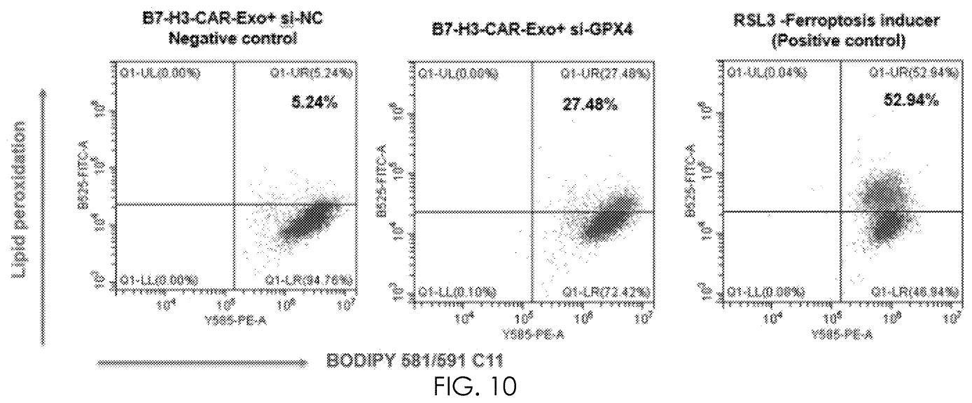

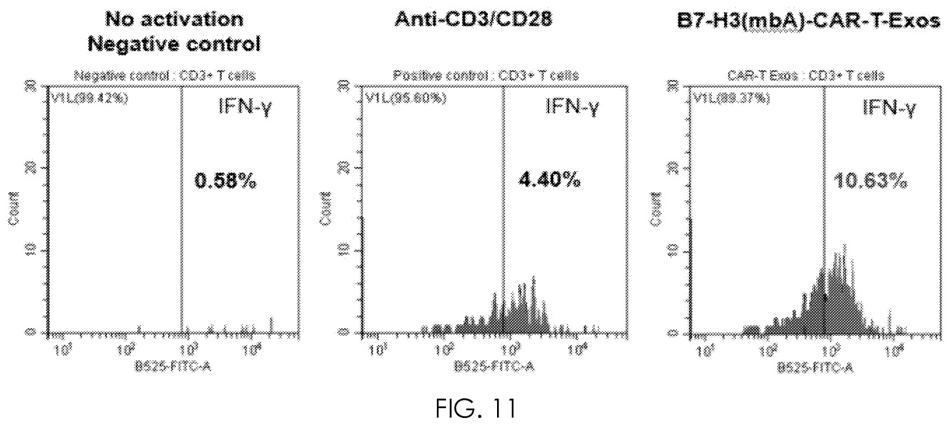

MB has a poor response to conventional chemotherapy and immunotherapy. CAR-T cell-derived exosomes (CAR-Exo) drive significant anti-tumor effects with low immunotoxicity, can be targeted to specific tumor cells, and can be loaded with therapeutic cargoes⁴⁵. Our screening of clinical data from patients with medulloblastoma and MB cell lines revealed that the CD276 (B7-H3) immunoregulatory oncogene is significantly overexpressed in pediatric MB, particularly in G3-MB, compared with normal cerebellum (FIG. 6). CD276 (B7-H3) has previously been used as a target for CAR-T cells in several pediatric and adult brain tumors like MB, neuroblastoma, ependymoma, and glioblastoma⁸¹. GPX4 inhibits ferroptosis and induces tumor growth⁸², and its overexpression has been reported in several cancers, especially glioma⁸³. We found GPX4 is highly expressed in G3-MB compared with other MBs and normal cerebellum (FIG. 7). We transduced anti-B7-H3(mAb)-CAR-GFP into sorted human T cells and detected expression of the anti-B7-H3-CAR (mAb) by flow cytometry (FIG. 8). These anti-B7-H3-CAR T cells were used to isolate anti-B7-H3-CAR-T exosomes, which expressed anti-B7-H3-CAR (mAb) on their surface as validated by flow cytometry. CAR-Exos were confirmed using nanoparticle tracking analysis (NTA) and western blotting for exosome-specific protein markers. In vitro, CAR-Exos efficiently targeted D425 cells expressing B7-H3 (FIG. 9). Furthermore, anti-B7-H3-CAR-T-Exo were loaded with si-GPX4, which knocked down GPX4 at mRNA and protein levels. CAR-Exos can stimulate tumor cell death due to IFN-7 and perforin, which induce tumor cell apoptosis and ferroptosis⁸⁴. However, loading si-GPX4 into anti-B7-H3-CAR-Exos amplified tumor cell death due to the induction of ferroptosis (FIG. 10). Furthermore, the addition of anti-B7-H3-CAR-Exos to naïve T cell cultures induced T cell activation and proliferation (FIG. 11) together with the release of IFN-γ, indicating significant T cell activation compared with negative control. Therefore, we hypothesize that loading si-GPX4 into anti-B7-H3-CAR-Exos will provide a powerful, synergistic immunotherapy that can cross the BBB to specifically target MBs.

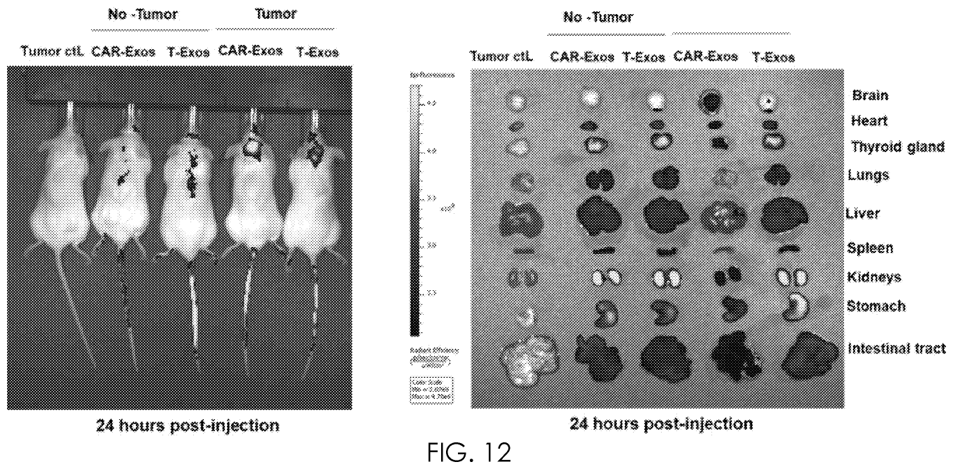

Tracing the delivery of B7-H3-CAR-Exos into NOD/SCID bearing MBs using CAR-Exos labeled with Cy5.5 showed accumulation of B7-H3-CAR-Exos in brain tumor tissue in the mouse cerebellum (FIG. 12). After tail vein injection of CAR-Exos, there was significant accumulation of CAR-Exos in brain tumors, suggesting that CAR-Exos efficiently cross the BBB and specifically accumulate in tumor tissue. The efficacy of B7-H3 (mbA)-CAR-Exo-si-GPX4 in treating MBs by inducing tumor ferroptosis and anti-tumor immune responses without immunotoxic side effects will be investigated.

### Methodology

B7-H3(mbA)-CAR-T-Exo generation: Construction of the B7-H3-CAR lentiviral vector: Briefly, the pCDCAR1 cloning vector is used to construct the lentivirus vector that sequentially contains a mouse anti-human B7-H3(mbA) single-chain variable fragment sequence (scFv), CD8 hinge and transmembrane domains, and the intracellular signaling domains of 4-1BB and CD3ζ intracellular domain. B7-H3-CAR will be connected to GFP by a p2A peptide to generate membrane-bound GFP (mGFP). B7-H3(mbA)-CAR-GFP will be inserted into a plasmid pCDCAR1 to produce a lentiviral vector by transducing 293T cells. The particles will be aliquoted and stored at −80 °C.

Generating anti-B7-H3(mbA)-GFP-CAR-T cells and harvesting ant-B7-H3(mbA)-CAR-T exosomes: Human CD3⁺ T cells will be sorted from PBMCs obtained from healthy donors using negative selection kits from Stemcell. Then, CD3⁺ cells will be tested by flow cytometry to confirm purity. CD3⁺ T cells will be cultured and expanded in ImmunoCult-XF T cell Expansion Medium from Stemcell supplemented with soluble anti-CD3/anti-CD28 activator and IL-2⁸⁵. After 3 days of CD3⁺ T cell activation, cells will be transduced with B7-H3(mbA)-CAR lentivirus. The expression of B7-H3(mbA)-CAR will be assessed by flow cytometry. Then, B7-H3(mbA)-CAR exosomes will be harvested from the medium of CAR-T cells using differential ultracentrifugation (SW32Ti Rotor, 5000 rpm for 10 min; 10,000 rpm for 10 min; 25,000 rpm for 120 min) and size exclusion chromatography for purification. The isolated B7-H3(mbA)-CAR exosomes will be quantified and characterized using NTA, TEM, flow cytometry, and western blotting. B7-H3(mbA)-CAR exosomes will be stored at −80 °C for further experiments.

Investigating anti-tumor immune responses to CAR-T Exosomes therapy: Testing the capability of B7-H3 (mbA)-CAR-T cell-derived exosomes to induce tumor toxicity and cell death with without si-GPX4: In our preliminary experiments, we used pCDCAR1-Anti-B7-H3 (mAb 1) h(CD28-OX40-CD3ζ) CAR lentivirus vector to transduce sorted CD3⁺ T cells isolated from the PBMC fraction of healthy human donors (ethical approval IRB00175372) and then generated anti-B7-H3-CAR T cells. CAR-Exo were isolated from these cells using ultracentrifugation and purification⁸⁶. MB cell lines (D425, MED-211, CHLA01, DAOY) will be used in this experiment. (a) 1×10⁴ tumor cells expressing GFP will be cultured in 48-well plates for 24 hours. Then, 100-150 μg of B7-H3(mbA)-CAR-Exos+si-GPX4, B7-H3(mbA)-CAR-Exos+si-NC, and T-Exos will be injected into tumor cell cultures and tumor cell death rates visualized at 12, 24, 36, 48, 60, and 72 h. (b) The same experiment will be used to check GFP positivity 24, 48, and 72 h after treatment by flow cytometry to assess relative cell viability. (c) Other than the ferroptosis endpoints listed in SA1, cell cycle analysis will be performed at the same time points using a Cell Cycle Analysis Kit.

Testing potential CD8⁺ T cell differentiation and proliferation in response to B7-H3(mbA)-CAR-Exos+/−si-GPX4: Human primary T cells will be isolated from healthy donor PBMCs to test T cell differentiation and proliferation responses to CAR-T-Exos. T cells will be stained with CFSE following a published protocol⁸⁷. Next, 1×10⁵ stained T cells will be plated into 96-well plates and 100-150 μg B7-H3(mbA)-CAR-Exos±si-GPX4 added, with soluble anti-CD3/CD28 activator+IL-2 as a positive control. After incubating for 5-6 days, T cells will be collected, washed, and stained with a viability dye and anti-CD4, anti-CD8, and anti-IFN-7 antibodies. T cell subsets will be analyzed by flow cytometry, and their differentiation and proliferation in response to B7-H3(mbA)-CAR-Exos±si-GPX4 treatment will be quantified.

Testing potential activation and exhaustion biomarkers in response to treatment with B7-H3(mbA)-CAR-Exos+/−si-GPX4: T cell activation and exhaustion in response to CAR-Exos will be assessed. 1-2×10⁵ PBMCs from healthy donors will be cultured in 48 wells for 24 hours, after which 100-150 μg B7-H3(mbA)-CAR-Exos±si-GPX4 will be added. Cells will be incubated for 3-5 days and then stained with viability dye, anti-CD3, anti-CD4, anti-CD8, anti-PD1, anti-TIM3, anti-LAG3, and anti-CTLA4 antibodies, with intracellular staining for IFN-γ, IL-17, and FOXP3 performed according to protocol. Finally, immune cells will phenotyped by flow cytometry.

Identify tumor cell responses to CAR-T exosome therapy: The effect of B7-H3 (mbA)-CAR-T-Exo with/without si-GPX4 on tumor cell growth and proliferation will be examined in 1×10⁴ tumor cells (D425) plated per well. 100 μg CAR-T-Exos will be injected into tumor cell cultures and incubated for 24, 48, 72, and 96 h and cell viability assessed using the MTS assay⁸⁸.

Cell cycle analysis of D425 tumor cells treated with B7-H3 (mbA)-CAR-T-Exo with/without si-GPX4 will be performed by plating cells into 24-well plates (1×10⁵ cells/well). Then, B7-H3 (mbA)-CAR-T-Exo with/without si-GPX4 will be added to the tumor cell culture. The tumor cell cycle will be tested at 24, 48, and 72 h using a cell cycle kit and flow cytometry.

Investigating anti-tumor effects of B7-H3(mbA)-CAR-T Exosome therapy in vivo: Humanized 4-6-week HuCD34-NCG mice will be used to establish an orthotopic MB model. Tumor growth will be monitored by IVIS⁸⁸. Once the tumor signal becomes detectable, tail vein injections of B7-H3(mbA)-CAR-Exos+si-GPX4 or B7-H3(mbA)-CAR-Exos+si-NC will be performed once a week for 8 weeks. Control groups will be untreated tumors, B7-H3(mbA)-CAR-Exos only, naked si-GPX4, and naked si-NC. (1) Tumor growth will be monitored once a week using the IVIS system until the control group reaches the endpoint. MRI imaging will be performed to confirm tumor size in all groups treated with CAR-Exo+si-GPX4 compared with controls. (2) During treatment, mouse behavior and movement or any possible disabilities will be recorded. (3) Mice in all groups will euthanized and brains collected, with tumor samples excised from adjacent tissues. Tumor size and volume will be automatically measured in all groups using a Peira tumor scanner. (4) Tumor samples will be divided as follows: (a) total RNA and protein will be collected to check the expression of target genes using qRT-PCR and western blotting; (b) part of tumor samples will be frozen to assess single cell populations and signals activated by CAR-Exos-siRNA therapy; (c) part of frozen tumors will be subjected to immunofluorescence staining for visualizing immune cell populations in the tumor microenvironment to explore immune responses to therapy; (d) Histopathological studies will be performed to score response to treatment in mice based on differentiation markers like GFAP, synaptophysin, and IMSM1 as well as proliferation (Ki67). In addition, we will measure iron, ROS, and lipid peroxidation levels observed using BODIPY-C11 probe and flow cytometry. (e) Immune cells will be isolated from tumor samples using a CD45⁺ selection kit and then CD8⁺/CD4⁺ selection kits to validate transcriptomics data. (4) Blood samples will be collected from all mice, and the levels of pro-inflammatory cytokines associated with CRS⁸⁹ like IL-6, soluble IL-6R, IFN-γ, IL-2, soluble IL-2Rα, IL-6, granulocyte-macrophage colony-stimulating factor (GM-CSF), and IL-32 will be measured by ELISA.

Expected outcomes and alternative strategies: Our hypothesis and supporting preliminary data expect promising tumor elimination and anti-tumor immune responses by B7-H3(mbA)-CAR-Exos enriched with si-GPX4. GPX4 is a well-known ferroptosis suppressor, and targeting this gene initiates tumor cell death by ferroptosis in many tumor types. Our preliminary data supports the same effect in MB. Furthermore, this type of biological delivery is specific only for tumor cells overexpressing the B7-H3 oncogene on the surface, which will reduce any potential side effects of siRNA therapy in normal tissue. Moreover, our data show that B7-H3(mbA)-CAR-Exos crosses the BBB and specifically targets tumor tissue. These encouraging results suggest the feasibility of this technique and its promising novelty. Alternative strategies include: (1) as seen in our preliminary data, CAR-Exos can efficiently be loaded with more than one si-RNA, so loading CAR-Exos with multiple siRNAs (such as si-ACLS4, as well as IL-2 to promote immune responses) is a possibility to enhance efficacy; (2) searching of clinical databases of pediatric MB showed interesting overexpression of the EPHA2 oncogene in group 3 MB, so generating EPHA2 (mbA)-CAR-T-Exo is an alternative strategy to the B7-H3 target; (3) recent reports discuss the capability of exosomes to deliver small chemotherapeutic drugs, which is another possibility for enhancing efficacy dose twice a week. SHH mouse model 136; 129T2-Ptch1^{tm1Bjw}/WreyJ will be used as a backup to develop proof-of-principle studies.

All animal experiments will be performed on equal numbers of female and male animals (see the overall statistical plan for mouse numbers). One-way ANOVA will be used to determine significant differences between groups regarding treatment outcomes. The Holm-Sidak method will be used for multiple pairwise comparisons. Ten mice per group are necessary to detect a 25% difference in the means by ANOVA with a standard deviation of 10% based on previous studies with a power of 0.8, alpha of 0.05. Significance will be accepted at a P<0.05 using nonparametric tests, and confidence intervals will be reported. Male and female mice will be used in equal numbers, with enough in each category for statistical significance. Kaplan-Meier graphs with log-rank analysis will determine if there is a significant impact on survival in the experimental versus test groups.

### Conclusion

Our preliminary and published results support the hypothesis that GPX4 is an excellent therapeutic target in MB. Our proposed studies will determine (i) the mechanism by which the miR-211-GPX4-ACSL4 axis promotes MB tumorigenesis; (ii) an in-depth analysis of miR-211-driven molecular and cellular changes to pave the way for therapeutic targeting; (iii) the potential efficiency of miR-211-associated genes to enhance the response of MB to specific immunotherapy; (iv) in vivo preclinical testing of the efficacy and safety of a cutting-edge RNA therapeutic agent alone and in combination with standard-of-care; and (v) contextualization of miR-211-GPX4-ACSL4 axis within wider signaling networks to provide alternative targets and opportunities to overcome emergent resistance.

### Example 3: Develop a Novel Immunotherapeutic Approach Based on Cd276 (B7-H3)-Car-T Cell-Derived Exosomes Harboring MIR-211

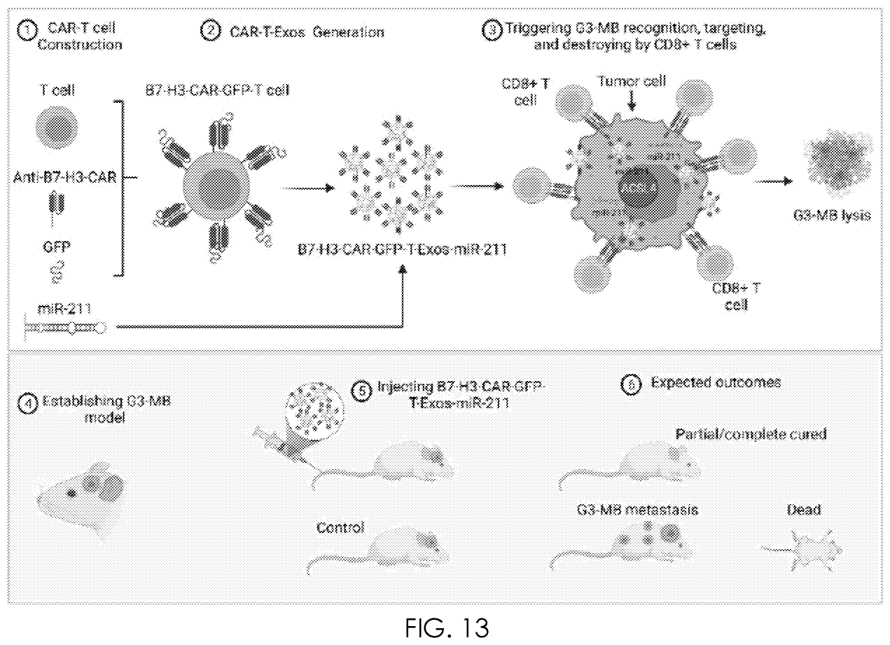

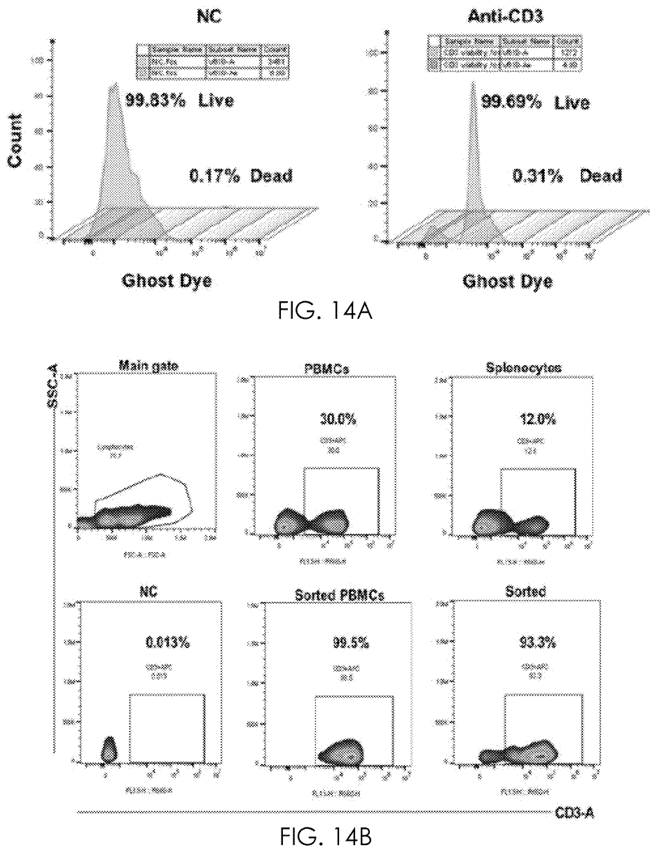

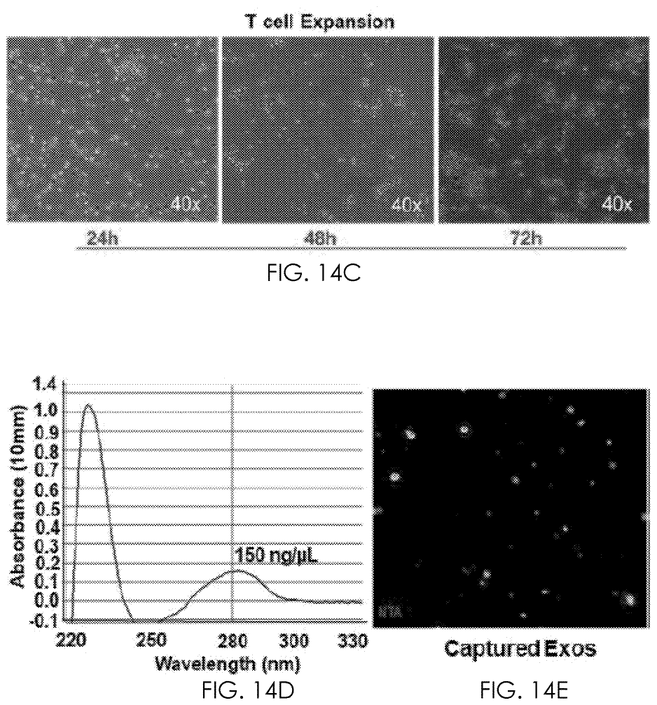

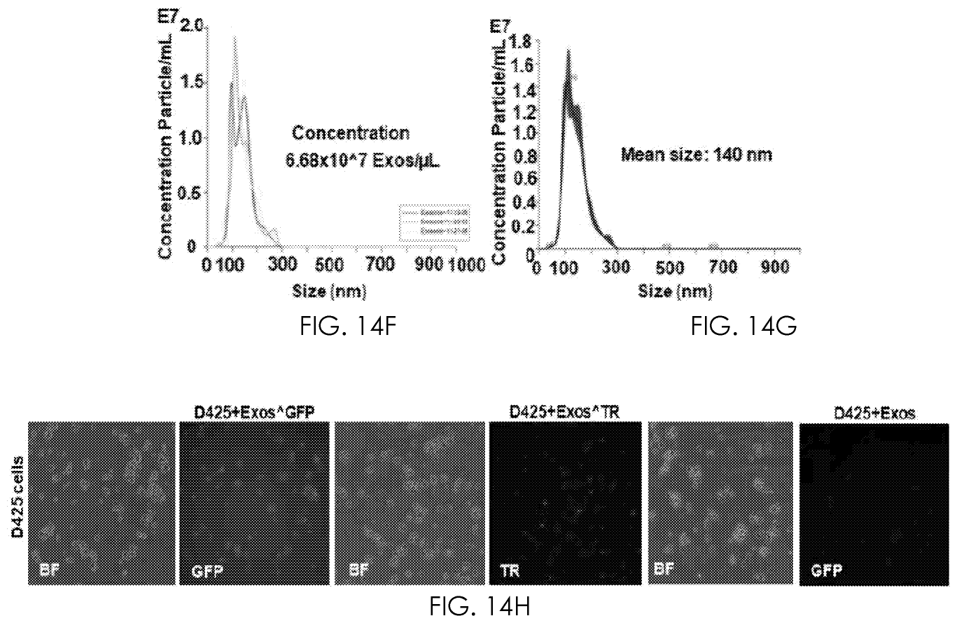

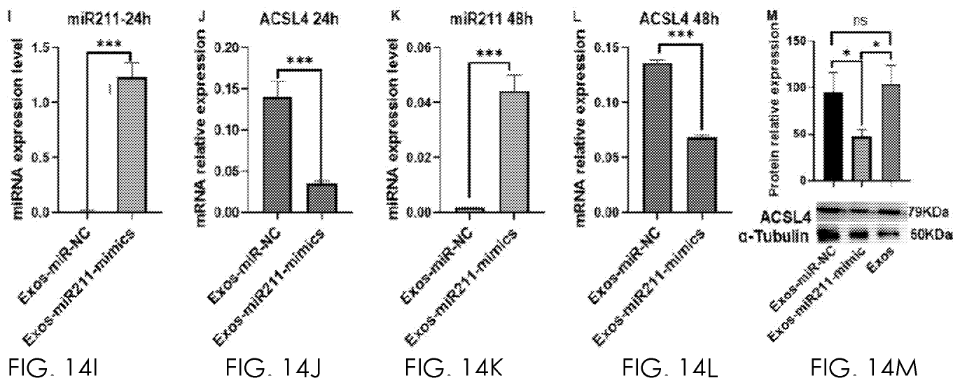

MB has a poor response to conventional chemotherapy and immunotherapy. CAR-T cell-derived exosomes (CAR-Exo) show significant anti-tumor immune responses with low immunotoxicity. We hypothesize that combining miR-211 and B7-H3-CAR-Exos will provide a powerful, synergistic targeted immunotherapy against MBs that can downregulate ACSL4 and enhance specific antitumor immune responses, as presented in FIG. 13. Unlike CD19 and CD22, lineage-derived antigens are highly expressed on cancer cells⁶⁴. Exosomes have the advantage of being able to penetrate tumor cells to release cargo. Since B7-H3-CAR T cell therapy has shown promise against glioma and MB^{65, 66}, we postulate that a combined B7-H3-CAR-Exo-miR-211 system would synergize to induce anti-tumor immune responses without immunotoxicity. B7-H3 is a 316 amino acid-long type I transmembrane protein encoded by a gene on chromosome 9 in mice and at 15q24 in humans. It exists in two isoforms determined by its extracellular domain. In mice, the extracellular domain comprises a single pair of immunoglobulin variable (IgV)-like and immunoglobulin constant (IgC)-like domains, whereas in humans, it comprises one or two identical pairs due to exon duplication^{67, 68}. In this Aim, we will investigate the efficacy of B7-H3-CAR-Exo-miR-211 in inhibiting MB growth by intracellular targeting of the ACSL4 oncogene, inducing ferroptosis, inducing CD8⁺ T cell responses by B7-H3-CAR, guiding Exo to the target by B7-H3-CAR, and avoiding T cell exhaustion and immunotoxicity adverse events. In our preliminary studies, T cell-Exo delivery efficiency was assessed by transfecting T cell-Exos with EGFP and Texas red in D425 cells. Transfected cells expressed fluorescent protein in 48 hours. T-cell-Exos-miR-211 successfully delivered miR-211 to D425 cell lines and downregulated the expression of ACSL4 significantly (FIG. 14). This study will use a humanized mouse model (HuCD34-NCG) and the medulloblastoma SHH mouse model developed by one of our Co-Is.

### Methodology

The process of loading miR-211 and TR-siRNAs into exosomes. Exo-Fect exosome transfection kit from System Biosciences will be used to load T-cell-Exos with miR-211, miR-NC, EGFP, and Texas Red siRNA. Briefly, 20 nmol of every agent will be mixed with 5 ug of exosome (approximately 1×10⁷) and 10 ul Exo-Fect reagent will be added. The reaction will be incubated at 37 °C with shaking for 10 min and then immediately transferred on ice. 30 ul of Exo-Quick-TC reagent will be added for every reaction to stop transfection and then incubate on ice for 30 min. Next, all tubes will be centrifuged at 15000 rpm/5 min. The supernatant will be discarded, and pellets will be suspended in 300 ul of sterile PBS. Centrifugation will be repeated to remove any residual buffers. After that, the transfected Exos will be suspended into 150 ul of RPMI medium. Finally, Exos loaded miR-211, miR-NC, EGFP, and TR-siRNA will be injected into the D425 cell line culture and incubate for 24 hs, 48 hs, and 72 hs. Total RNA will be extracted from 24 h and 48 h and the expression of miR-211 levels and ACSL4 will be tested by qPCR. The protein expression level will be tested by western blotting from cells that incubated 72 hrs. The fluorescent of GFP and TR-siRNA will be visualized by fluorescent microscopy after 48 hs and 72 hrs.

Construction of anti-B7-H3-CAR lentiviral vector. The anti-B7-H3-CAR construct sequentially contains a mouse anti-human B7-H3 single-chain variable fragment sequence (scFv), CD8 hinge and transmembrane domains, and the intracellular signaling domains of 4-1BB and CD3ζ intracellular domain. B7-H3-CAR will be connected to GFP by a p2A peptide to generate membrane-bound GFP (mGFP). B7-H3-CAR-GFP will be inserted into a plasmid pRRL-B7-H3-CAR-GFP to produce a lentiviral vector by transducing 293T cells. The particles will be aliquoted and stored at −80 °C.

Isolating CD8+ T cells, exosomes, and characterizing B7-H3-CAR-T-Exos.

We will isolate CD8⁺ T cells from PBMCs from healthy donors using a positive selection kit and CytoFLEX SRT sorter. CD8⁺ T cells will be cultured for 72 hours in RPMI-1640 supplemented with FBS-free exosomes and Dynabeads™ labeled with CD3/CD28. CD8⁺ T cells will be transduced with GFP-B7-H3-CAR lentivirus to generate GFP-B7-H3-CAR-T cells, with flow cytometry used to assess CAR expression. GFP-B7-H3-CAR-T cells will be cultured for 72 hours in RPMI-1640 supplemented with FBS-free exosomes and Dynabeads™ labeled with CD3/CD28. Exosomes will be collected by differentialultra-centrifugation; (5000 rpm/5 min; 10,000 rpm/10 min; 25,000 rpm/120 min) using an SW 32 Ti rotor; purified by size exclusion chromatography; and characterized by flow cytometry, nanoparticle tracking analysis (NTA), transmission electron microscopy (TEM), and western blotting using CD9, CD63, and CD81 antibodies.

Co-culturing MB cell lines with GFP-B7-H3-CAR-T-Exo-miR-211. We will examine: (a) the delivery of GFP-B7-H3-CAR-T-Exo-miR-211 into tumor cells using immunofluorescence microscopy and confocal microscopy; (b) the expression of ACSL4 in tumor cells using qRT-PCR and western blotting; (c) the expression of miR-211 in transfected tumor cells; (d) changes in tumor cell phenotype (ferroptosis, colony formation, proliferation, cell cycle, and apoptosis); (e) identify the cell population entering ferroptosis.

Next, CD8+ T cells from healthy donors will be co-cultured with GFP-B7-H3-CAR-T-Exos-miR-211 for 72 hours, and the differentiation and proliferation of cytotoxic CD8+ T cells will be examined by flow cytometry for surface markers and ELISA for released cytokines. Activated cytotoxic CD8+ T cells will be selectively isolated and co-cultured with MB cell lines for 24 hours, with the activity of differentiated CD8+ T cells examined using fluorescence imaging. The efficacy of CD8+ T cells against MB cells in the presence and absence of GFP-CAR-T-Exo-miR-211 will be examined with apoptosis and cell cycle of tumor cells measured as endpoints by confocal microscopy, Annexin-V/PI staining, PI, and flow cytometry. We will also quantify tumor cell proliferation and cytokine release into the supernatant using ELISA, the cell trace proliferation kit, flow cytometry, qRT-PCR, and immunofluorescence microscopy. MB cell lines (SHH, G3, and G4) will be co-cultured with CD8+ T cells and GFP-B7-H3-CAR-T-Exos-miR-211 in a 3D culture system, and the performance of activated CD8+ T cells in targeting tumor cells and preventing colony formation will be examined.

In vivo studies. We will establish a humanized MB mouse model (HuCD34-NCG mice, 4-5 weeks) and divide mice into six groups: PBS, CAR-T cell, B7-H3-CAR-T cell, B7-H3-CAR-T-Exo, B7-H3-CAR-T-Exo-miR-211, and miR-211 treatments. miR-211 will be labeled with Cy3. When the tumor reaches an average of 100 mm³, multiple doses weekly will be injected intravenously (twice/week) for one month and two months. Tumor size/volume will be monitored during treatment and drug distribution via Cy3-labeled miR-211. All mice will be sacrificed, and then tumors, blood, spleen, lymph nodes, and other organs including lungs, kidneys, and bones will be observed. The tumor samples will be divided into two, with one embedded in paraffin for IHC and another frozen for immunofluorescence analyses. Blood, spleen, and lymph nodes will be used to evaluate the ratios of effector CD8+ T cell populations and active markers together with the populations of other activated immune cells. Plasma samples will be used to investigate cytokine levels and the presence of cytokine release syndrome.

Expected outcomes and alternative strategies. In this Aim, miR-211-enriched B7-H3-CAR-Exos will guide and stimulate immune responses to the tumortissue and at the same time downregulate ACSL4 expression in tumor cells leading to induce cell death. When Treating MBs with engineered T cells, truly MB-specific surface targets remain a major barrier. Targeting molecules expressed in both MBs and normal cells can cause life-threating toxicity as observed in clinical trials with CAR-T cells targeting epidermal growth factor receptor 2. Therefore, using CAR-T exosomes is the alternative safe strategy as reported with no signs of toxicity, even at the highest dose²³. Hence, we expect that exosomes will pass the blood brain barriers and modulate immune cell populations at the tumor tissue leading to enhance CD8+ T cell differentiation and killing. On the other hand, we will carefully monitor the immunotoxicity events such as cytokine release syndrome and neurological disorders. We will measure the high level of immune activation by assaying circulating cytokines including but not limited to IL-6, IL-12, IL-18, IL-17, TNF-α and IFN-γ. In case of signs of immunotoxicity observed, one dose of exosomes loaded miR-211 will be injected at tumor position to deliver miR-211 to the tumor tissue and testing brain anti-tumor immune responses.

We will use unbiased approaches to investigate the role of miR-211 in medulloblastoma growth and development and perform independent experiments multiple times to guarantee that we pursue only robust observations. Throughout the proposal, we discuss potential problems, alternative strategies, and our plans to address biological variables rigorously. All animal experiments will be performed on equal numbers of female and male animals (see the overall statistical plan for mouse numbers). One-way ANOVA will be used to determine significant differences between groups regarding treatment outcomes. We will use the Holm-Sidak method for multiple pairwise comparisons. Ten mice per group are necessary to detect a 25% difference in the means by ANOVA with a standard deviation of 10% based on our previous studies with a power of 0.8, alpha of 0.05. Significance will be accepted at a P<0.05 using nonparametric tests, and we will report confidence intervals. We will use male and female mice in equal numbers, with enough in each category for statistical significance. Kaplan-Meier graphs with log-rank analysis will determine if there is a significant impact on survival on the experimental versus test groups.

Conclusion. Our preliminary and published results support the hypothesis that miR-211 is an excellent therapeutic agent that offers promising opportunities for diagnosis and therapy of MBs. Our proposed studies will determine (i) the mechanism by which miR-211 promotes MB tumorigenesis; (ii) an in-depth analysis of miR-211-driven molecular and cellular changes to pave the way for therapeutic targeting; (iii) the potential efficiency of miR-211 to enhance the response of MB to specific immunotherapy; (iv) in vivo preclinical testing of the efficacy and safety of cutting-edge RNA therapeutic agent alone and in combination with standard of care; and (v) contextualization of miR-211 within wider signaling networks to provide alternative targets and opportunities to overcome emergent resistance.

### Example 4: Develop a Novel Immunotherapeutic Approach Based on Cd276 (B7-H3)-Car-T Cell-Derived Exosomes Harboring SI-HLX

Medulloblastoma (MB) is the most common malignant brain tumor in children. Treatment often involves aggressive multimodal therapy, resulting in significant long-term side effects. Group 3 medulloblastomas (G3 MBs), the most aggressive molecular subgroup, are associated with treatment-resistant recurrences, high morbidity, and mortality. Despite advances in molecular characterization of other MB subgroups (e.g., Wnt- and Shh-activated MBs), the genetic and molecular drivers of G3 MBs remain poorly understood, and effective targeted therapies are lacking. Our preliminary studies have identified the H2.0-like homeobox transcription factor (HLX) as a promising therapeutic target for G3 MBs. HLX is significantly upregulated in G3 MB patient samples and cell lines, and its depletion reduces tumor growth in vitro and in vivo. Furthermore, HLX regulates key oncogenic pathways, including the MYC-IGF2BP3 axis, and modulates tumor metabolism through the regulation of oxidative phosphorylation and mitochondrial dysfunction-related genes. Importantly, our preliminary studies show that lipid nanoparticle (LNP)-coated siHLX (LNP/si-HLX), which crosses the blood-brain barrier (BBB), reduces G3 MB xenograft tumor growth. We hypothesize that the HLX-MYC-IGF2BP3 axis is a key driver of G3 MB progression and a targetable therapeutic pathway. Additionally, HLX-mediated metabolic reprogramming contributes to tumor growth and plasticity, offering novel opportunities for therapeutic intervention.

Identify HLX-dependent metabolic dependencies in G3 MB that can be exploited for combinatorial approaches. HLX modulates tumor metabolism, acting as a metabolic switch to regulate oxidative phosphorylation and mitochondrial dysfunction. We hypothesize that uncovering HLX-dependent metabolic reprogramming could expose new therapeutic vulnerabilities and potential drug targets in G3 MB. Functional analyses of HLX-regulated metabolic pathways (e.g., oxidative phosphorylation, glycolysis, and fatty acid metabolism) are performed using CRISPR knockdown and metabolic inhibitors. In addition, how HLX-mediated downregulation of electron transport chain (ETC) genes contributes to mitochondrial dysfunction, the tumor microenvironment, and tumor progression is identified. The present inventors expect to identify metabolic pathways regulated by HLX and establish their roles in tumor growth, heterogeneity, and plasticity, providing additional therapeutic targets for G3 MB.

Develop combination strategies for G3 MB by targeting and inhibiting the HLX pathway. HLX is overexpressed in G3 MBs and promotes tumor growth. We hypothesize that delivering siHLX using Car-T cell derived exosomes that cross the BBB represents a promising therapeutic approach for G3 MBs. Car-T cell derived exosomes harboring si-HLX are formulated and optimized for BBB penetration and targeting tumors. The efficacy of Car-T cell derived exosomes loaded with si-HLX alone or in combination with cisplatin in immunocompromised mice bearing intracranial G3 MB xenografts is evaluated. To assess efficacy and safety, a syngeneic, immune-competent G3 MB mouse model generated by transplanting Myc/dominant-negative Trp53-transduced normal stem cells with or without HLX into immune-competent cerebella is used. The present inventors expect to establish preclinical safety, efficacy, and combinatorial potential of car-T cell derived exosomes loaded with si-HLX to support future clinical translation.

Identify the therapeutic significance and molecular mechanisms of the MYC-IGF2BP3 axis in G3 MB development. HLX regulates the oncogenic MYC-IGF2BP3 axis, contributing to G3 MB heterogeneity, tumor growth, and therapy resistance. We hypothesize that dual targeting of MYC and IGF2BP3 may provide synergistic anti-tumor effects.

Car-T cell derived exosomes harboring siRNAs targeting MYC and IGF2BP3 in xenograft and syngeneic models are developed and evaluated. CRISPR-Cas9 knock-down of MYC and IGF2BP3 in G3 MB cell lines are performed, followed by phenotypic assays (proliferation, apoptosis, and colony formation) and molecular profiling (ScRNA sequencing and Spatial transcriptomics). MYC-IGF2BP3 interactions are investigated using co-knockdown and rescue experiments to delineate their functional interplay. The present inventors expect to characterize the MYC-IGF2BP3 axis as a critical oncogenic driver and validate it as a therapeutic target for G3 MB.

By integrating molecular, therapeutic, and metabolic analyses, the present invention addresses the urgent need for targeted and personalized therapies medulloblastoma, in particular, for children with G3 MBs. These results will provide the foundation for clinical translation and improve outcomes for patients with this aggressive tumor subtype.

### Results

Interestingly, our data showed that the HLX gene a transcription factor that regulates cell growth is highly expressed in G3-MB and associated with poor prognosis. The siRNA-HLX inhibited tumor growth in vitro and in vivo and promisingly showed long survival in the immunocompetent mouse model. We developed CAR-T-Exosomes that can pass brain-blood barriers (BBB) and deliver therapeutic siRNA to the brain tumor. Further analysis revealed that CAR-T-Exosomes contain T cell cytotoxicity materials that can destroy tumor cells and induce anti-tumor immune response. Promisingly, the expression of HLX was significantly inhibited in the tumor cells and the anti-tumor immune response was enhanced. CAR-T-Exosomes have high safety and can induce anti-tumor response without stimulation of immunotoxicity side effects like cytokine release syndrome (CRS).

### Example 5: Develop a Novel Immunotherapeutic Approach Based on Cd276 (B7-H3)-Car-T Cell-Derived Exosomes Harboring SI-EPHA2

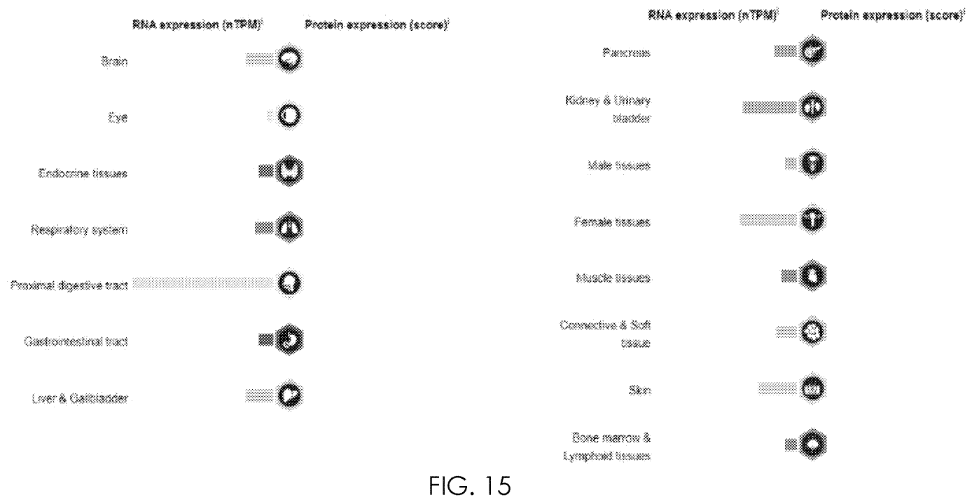

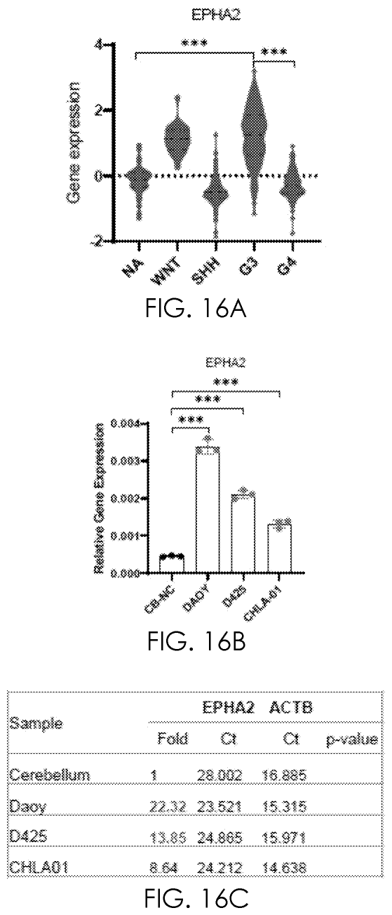

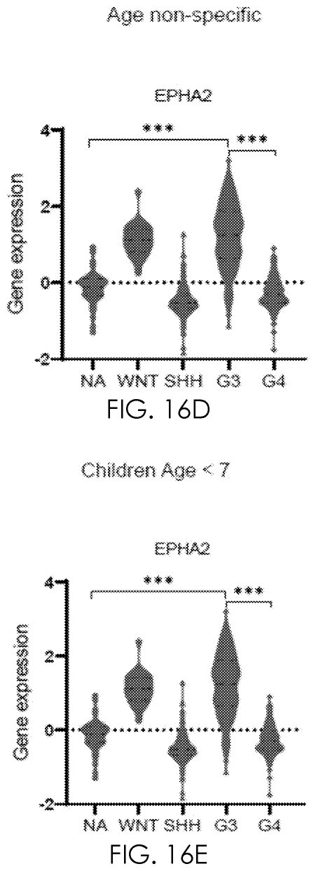

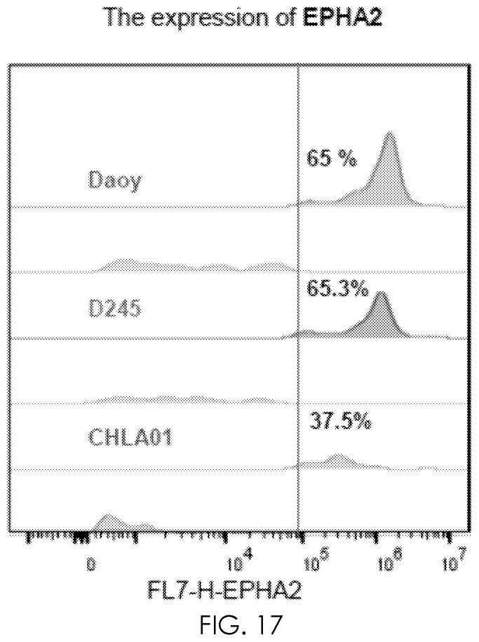

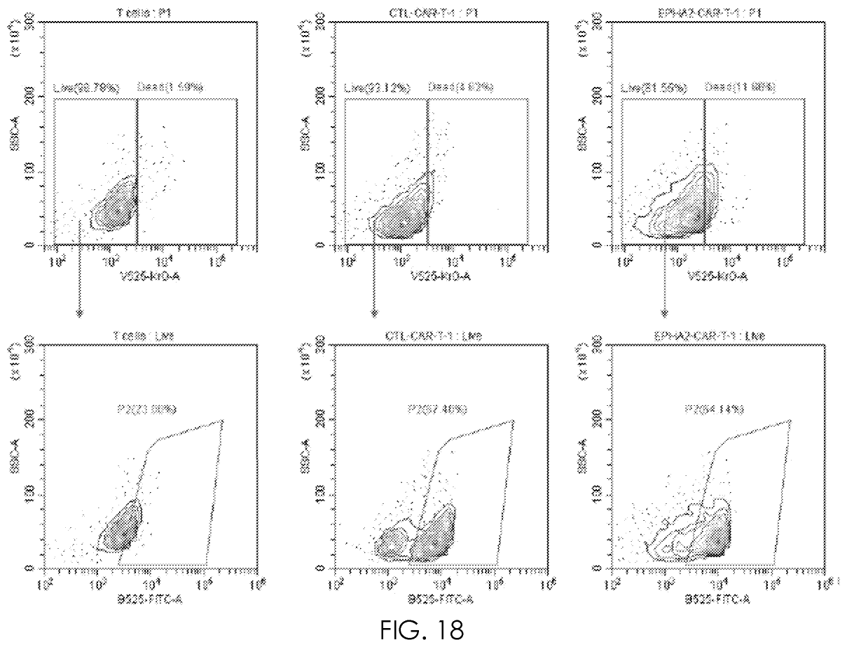

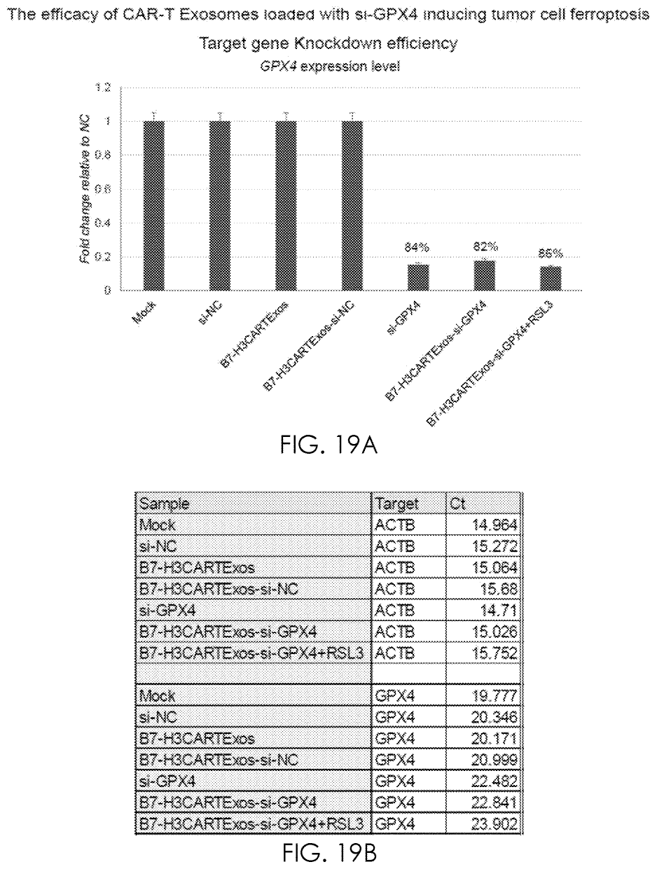

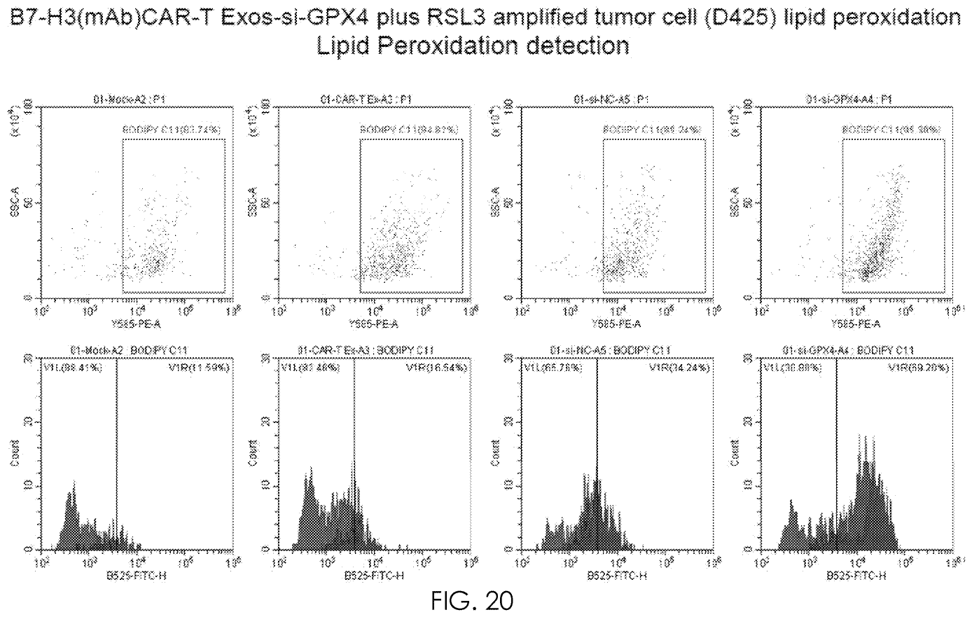

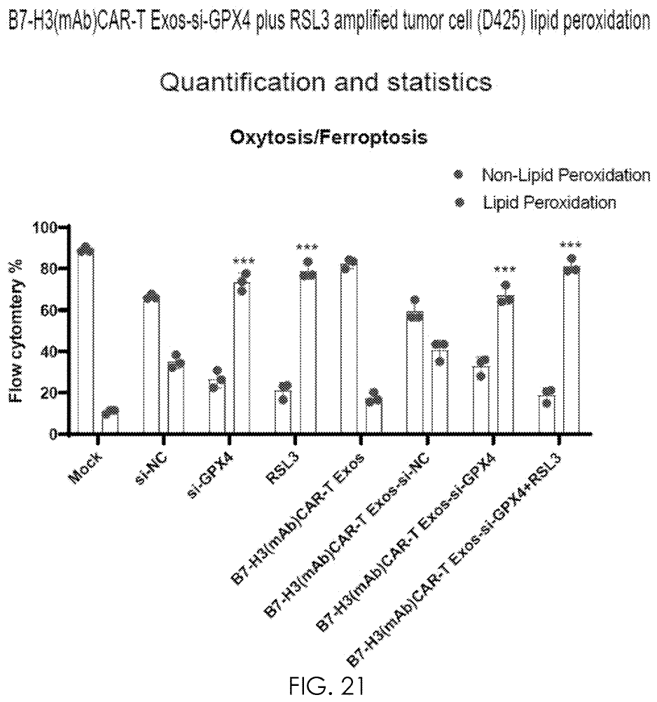

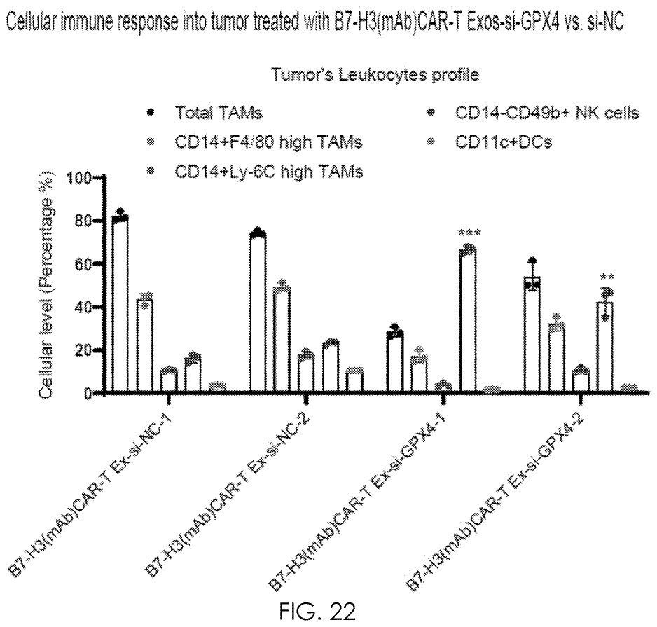

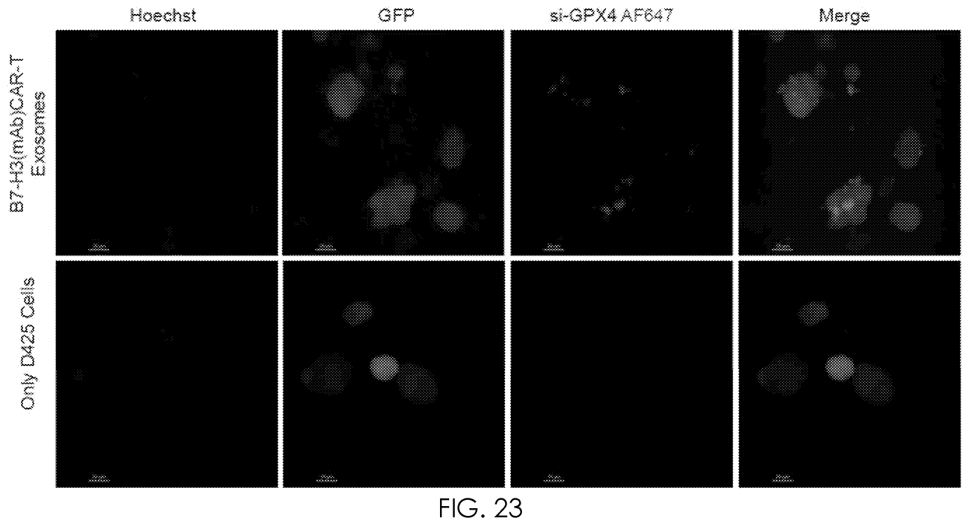

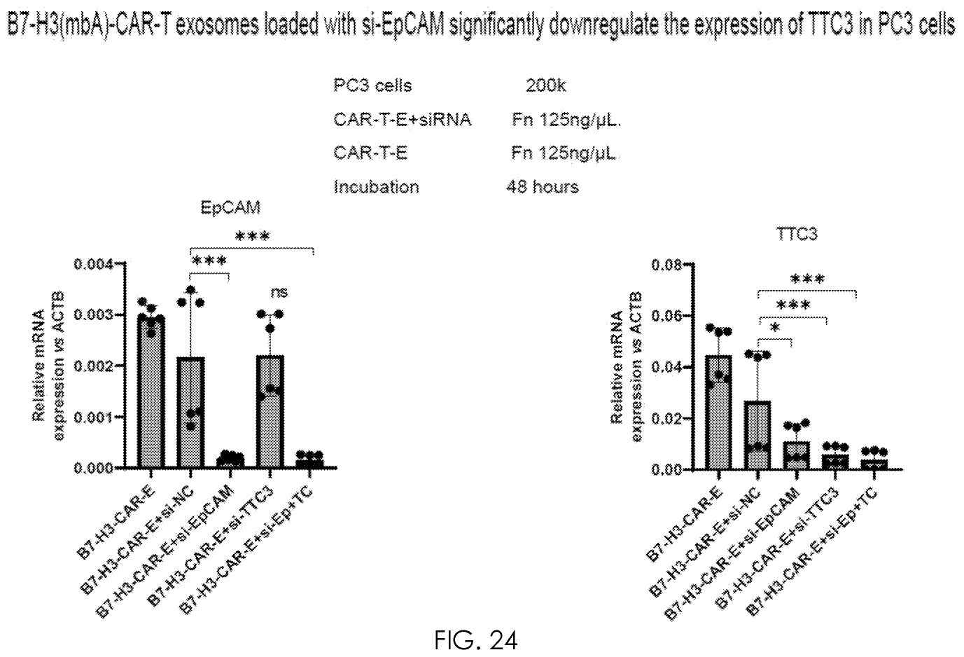

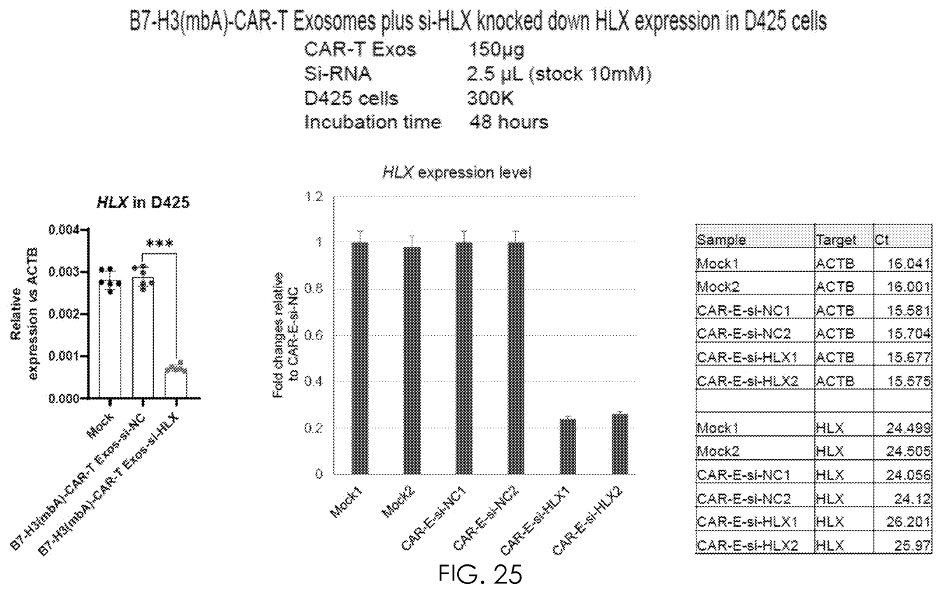

In this study, we are highlighting anti-EPHA2-CAR-Exosomes as a highly specific type of tumor CAR-T-Exos immunotherapy due to a very low expression of EPHA2 in normal cells and overexpression in medulloblastoma, particularly G3-MB. See FIGS. 15-18. Therefore, we speculate significant inhibition of tumor growth and activation of CTL effector cells in the tumor microenvironment. This study provides a new biological technique for delivering tumor suppressor RNAs and developing specific anti-tumor immune responses. Indeed, the promising safety of anti-EPHA2-CAR-T cell exosomes compared to cell-based immunotherapy.

That which is claimed:

1. A composition comprising an exosome isolated from a B7H3 chimeric antigen receptor (CAR) T-cell loaded with small interfering ribonucleic acids (siRNAs) to tetratricopeptide 3 (TTC3) and/or epithelial cell adhesion molecule (EpCAM).

2. The composition of claim 1, wherein the exosome is further loaded with siRNA to prostate cancer antigen 3 (PCA3).

3. The composition of claim 1, wherein the exosome is further loaded with a small molecule cancer therapeutic.

4. The composition of claim 1, further comprising a small molecular cancer therapeutic.

5. The composition of claim 3, wherein the small molecule cancer therapeutic comprises a polyadenosine diphosphate-ribose polymerase (PARP) inhibitor.

6. The composition of claim 5, wherein the PARP inhibitor comprises rucaparib, olaparib, talazoparib or niraparib.

7. A method for treating prostate cancer in a subject comprising the step of administering the composition of claim 1.

8. A composition comprising an extracellular vesicle isolated from a CAR T-cell loaded with a ribonucleic acid interference (RNAi) molecule.

9. The composition of claim 8, wherein the CAR comprises B7H3.

10. The composition of claim 8, wherein the extracellular vesicle comprises an exosome.

11. The composition of claim 8, wherein the RNAi molecule comprises siRNA.

12. The composition of claim 11, wherein the siRNA targets TTC3 and/or EpCAM.

13. The composition of claim 12, wherein the siRNA further targets PCA3.

14. The composition of claim 12, wherein the extracellular vesicle is further loaded with a small molecule cancer therapeutic.

15. The composition of claim 12, further comprising a small molecule cancer therapeutic.

16. The composition of claim 14, wherein the small molecule cancer therapeutic comprises a PARP inhibitor.

17. The composition of claim 16, wherein the PARP inhibitor comprises rucaparib, olaparib, talazoparib or niraparib.

18. A method for treating prostate cancer in a subject comprising the step of administering the composition of claim 8.

19. A composition comprising an exosome isolated from a B7H3 chimeric antigen receptor (CAR) T-cell loaded with small interfering ribonucleic acids (siRNAs) to glutathione peroxidase 4 (GPX4) and/or acyl-CoA synthetase long-chain family member 4 (ACSL4).

20. The composition of claim 19, wherein the exosome is further loaded with microRNA-211 (miR-211).

21. The composition of claim 19, wherein the exosome is further loaded with siRNAs to H2.0-like homeobox transcription factor (HLX), MYC, and/or insulin-like growth factor 2 mRNA-binding protein 3 (IGF2BP3).

22. The composition of claim 19, wherein the exosome is further loaded with a small molecule cancer therapeutic.

23. The composition of claim 19, further comprising a small molecular cancer therapeutic.

24. A method for treating prostate cancer in a subject comprising the step of administering the composition of claim 19.

25. The composition of claim 11, wherein the siRNA targets GPX4, ACSL4, HLX, MYC and/or IGF2BP3.

26. The composition of claim 25, wherein the extracellular vesicle is further loaded with miR-211.

27. The composition of claim 25, wherein the extracellular vesicle is further loaded with a small molecule cancer therapeutic.

28. The composition of claim 25, further comprising a small molecule cancer therapeutic.

29. The composition of claim 27, wherein the small molecule therapeutic comprises pemetrexed, gemcitabine, vismodegib, sonidegib, panobinostat, romidepsin, belinostat, CI-994 (tacedinaline), silmitasertib, palbociclib, sapanisertib, or a combination of palbociclib and sapanisertib.

30. A method for treating medullobastoma in a subject comprising the step of administering the composition of claim 19.


SEQUENCE LISTING (48 sequences)

SEQ ID NO: 1 - RNA, 21 nt, synthetic construct
  source 1..21: mol_type = other RNA
caatgccagtgtacttcagtt

SEQ ID NO: 2 - RNA, 21 nt, synthetic construct
  source 1..21: mol_type = other RNA
aactgaagtacactggcattg

SEQ ID NO: 3 - RNA, 19 nt, synthetic construct
  source 1..19: mol_type = other RNA
tgccagtgtacttcagttg

SEQ ID NO: 4 - RNA, 1547 nt, Homo sapiens
  source 1..1547: mol_type = mRNA
  CDS 196..1140: protein_id = 5; translation = MAPPQVLAFGLLLAAATATFAAAQEECVCENYKLAVNCFVNNNR QCQCTSVGAQNTVICSKLAAKCLVMKAEMNGSKLGRRAKPEGALQNNDGLYDPDCDES GLFKAKQCNGTSMCWCVNTAGVRRTDKDTEITCSERVRTYWIIIELKHKAREKPYDSK SLRTALQKEITTRYQLDPKFITSILYENNVITIDLVQNSSQKTQNDVDIADVAYYFEK DVKGESLFHSKKMDLTVNGEQLDLDPGQTLIYYVDEKAPEFSMQGLKAGVIAVIVVVV IAVVAGIVVLVISRKKRMAKYEKAEIKEMGEMHRELNA
acagagcgctagtccttcggcgagcgagcaccttcgacgcggtccggggaccccctcgtcgctgtcctcccgacgcggacccgcgtgccccaggcctcgcgctgcccggccggctcctcgtgtcccactcccggcgcacgccctcccgcgagtcccgggcccctcccgcgcccctcttctcggcgcgcgcgcagcatggcgcccccgcaggtcctcgcgttcgggcttctgcttgccgcggcgacggcgacttttgccgcagctcaggaagaatgtgtctgtgaaaactacaagctggccgtaaactgctttgtgaataataatcgtcaatgccagtgtacttcagttggtgcacaaaatactgtcatttgctcaaagctggctgccaaatgtttggtgatgaaggcagaaatgaatggctcaaaacttgggagaagagcaaaacctgaaggggccctccagaacaatgatgggctttatgatcctgactgcgatgagagcgggctctttaaggccaagcagtgcaacggcacctccatgtgctggtgtgtgaacactgctggggtcagaagaacagacaaggacactgaaataacctgctctgagcgagtgagaacctactggatcatcattgaactaaaacacaaagcaagagaaaaaccttatgatagtaaaagtttgcggactgcacttcagaaggagatcacaacgcgttatcaactggatccaaaatttatcacgagtattttgtatgagaataatgttatcactattgatctggttcaaaattcttctcaaaaaactcagaatgatgtggacatagctgatgtggcttattattttgaaaaagatgttaaaggtgaatccttgtttcattctaagaaaatggacctgacagtaaatggggaacaactggatctggatcctggtcaaactttaatttattatgttgatgaaaaagcacctgaattctcaatgcagggtctaaaagctggtgttattgctgttattgtggttgtggtgatagcagttgttgctggaattgttgtgctggttatttccagaaagaagagaatggcaaagtatgagaaggctgagataaaggagatgggtgagatgcatagggaactcaatgcataactatataatttgaagattatagaagaagggaaatagcaaatggacacaaattacaaatgtgtgtgcgtgggacgaagacatctttgaaggtcatgagtttgttagtttaacatcatatatttgtaatagtgaaacctgtactcaaaatataagcagcttgaaactggctttaccaatcttgaaatttgaccacaagtgtcttatatatgcagatctaatgtaaaatccagaacttggactccatcgttaaaattatttatgtgtaacattcaaatgtgtgcattaaatatgcttccacagtaaaatctgaaaaactgatttgtgattgaaagctgcctttctatttacttgagtcttgtacatacatacttttttatgagctatgaaataaaacattttaaactgaa

SEQ ID NO: 5 - protein, 314 aa, Homo sapiens
MAPPQVLAFGLLLAAATATFAAAQEECVCENYKLAVNCFVNNNRQCQCTSVGAQNTVICSKLAAKCLVMKAEMNGSKLGRRAKPEGALQNNDGLYDPDCDESGLFKAKQCNGTSMCWCVNTAGVRRTDKDTEITCSERVRTYWIIIELKHKAREKPYDSKSLRTALQKEITTRYQLDPKFITSILYENNVITIDLVQNSSQKTQNDVDIADVAYYFEKDVKGESLFHSKKMDLTVNGEQLDLDPGQTLIYYVDEKAPEFSMQGLKAGVIAVIVVVVIAVVAGIVVLVISRKKRMAKYEKAEIKEMGEMHRELNA

SEQ ID NO: 6 - RNA, 25 nt, synthetic construct
  source 1..25: mol_type = other RNA
gcagaagccagaatttgaattccct

SEQ ID NO: 7 - RNA, 25 nt, synthetic construct
  source 1..25: mol_type = other RNA
ctagcacacagcatgatcattacgg

SEQ ID NO: 8 - RNA, 25 nt, synthetic construct
  source 1..25: mol_type = other RNA
ccacaatatgcataaatctaactcc

SEQ ID NO: 9 - RNA, 3920 nt, Homo sapiens
  source 1..3920: mol_type = transcribed RNA
agaagaaatagcaagtgccgagaagctggcatcagaaaaacagaggggagatttgtgtggctgcagccgagggagaccaggaagatctgcatggtgggaaggacctgatgatacagaggaattacaacacatatacttagtgtttcaatgaacaccaagataaataagtgaagagctagtccgctgtgagtctcctcagtgacacagggctggatcaccatcgacggcactttctgagtactcagtgcagcaaagaaagactacagacatctcaatggcaggggtgagaaataagaaaggctgctgactttaccatctgaggccacacatctgctgaaatggagataattaacatcactagaaacagcaagatgacaatataatgtctaagtagtgacatgtttttgcacatttccagcccctttaaatatccacacacacaggaagcacaaaaggaagcacagagatccctgggagaaatgcccggccgccatcttgggtcatcgatgagcctcgccctgtgcctggtcccgcttgtgagggaaggacattagaaaatgaattgatgtgttccttaaaggatgggcaggaaaacagatcctgttgtggatatttatttgaacgggattacagatttgaaatgaagtcacaaagtgagcattaccaatgagaggaaaacagacgagaaaatcttgatggcttcacaagacatgcaacaaacaaaatggaatactgtgatgacatgaggcagccaagctggggaggagataaccacggggcagagggtcaggattctggccctgctgcctaaactgtgcgttcataaccaaatcatttcatatttctaaccctcaaaacaaagctgttgtaatatctgatctctacggttccttctgggcccaacattctccatatatccagccacactcatttttaatatttagttcccagatctgtactgtgacctttctacactgtagaataacattactcattttgttcaaagacccttcgtgttgctgcctaatatgtagctgactgtttttcctaaggagtgttctggcccaggggatctgtgaacaggctgggaagcatctcaagatctttccagggttatacttactagcacacagcatgatcattacggagtgaattatctaatcaacatcatcctcagtgtctttgcccatactgaaattcatttcccacttttgtgcccattctcaagacctcaaaatgtcattccattaatatcacaggattaacttttttttttaacctggaagaattcaatgttacatgcagctatgggaatttaattacatattttgttttccagtgcaaagatgactaagtcctttatccctcccctttgtttgattttttttccagtataaagttaaaatgcttagccttgtactgaggctgtatacagccacagcctctccccatccctccagccttatctgtcatcaccatcaacccctcccatgcacctaaacaaaatctaacttgtaattccttgaacatgtcaggcatacattattccttctgcctgagaagctcttccttgtctcttaaatctagaatgatgtaaagttttgaataagttgactatcttacttcatgcaaagaagggacacatatgagattcatcatcacatgagacagcaaatactaaaagtgtaatttgattataagagtttagataaatatatgaaatgcaagagccacagagggaatgtttatggggcacgtttgtaagcctgggatgtgaagcaaaggcagggaacctcatagtatcttatataatatacttcatttctctatctctatcacaatatccaacaagcttttcacagaattcatgcagtgcaaatccccaaaggtaacctttatccatttcatggtgagtgcgctttagaattttggcaaatcatactggtcacttatctcaactttgagatgtgtttgtccttgtagttaattgaaagaaatagggcactcttgtgagccactttagggttcactcctggcaataaagaatttacaaagagctactcaggaccagttgttaagagctctgtgtgtgtgtgtgtgtgtgtgagtgtacatgccaaagtgtgcctctctctctttgacccattatttcagacttaaaaacaagcatgttttcaaatggcactatgagctgccaatgatgtatcaccaccatatctcattattctccagtaaatgtgataataatgtcatctgttaacataaaaaaagtttgacttcacaaaagcagctggaaatggacaaccacaatatgcataaatctaactcctaccatcagctacacactgcttgacatatattgttagaagcacctcgcatttgtgggttctcttaagcaaaatacttgcattaggtctcagctggggctgtgcatcaggcggtttgagaaatattcaattctcagcagaagccagaatttgaattccctcatcttttaggaatcatttaccaggtttggagaggattcagacagctcaggtgctttcactaatgtctctgaacttctgtccctctttgtgttcatggatagtccaataaataatgttatctttgaactgatgctcataggagagaatataagaactctgagtgatatcaacattagggattcaaagaaatattagatttaagctcacactggtcaaaaggaaccaagatacaaagaactctgagctgtcatcgtccccatctctgtgagccacaaccaacagcaggacccaacgcatgtctgagatccttaaatcaaggaaaccagtgtcatgagttgaattctcctattatggatgctagcttctggccatctctggctctcctcttgacacatattagcttctagcctttgcttccacgacttttatcttttctccaacacatcgcttaccaatcctctctctgctctgttgctttggacttccccacaagaatttcaacgactctcaagtcttttcttccatccccaccactaacctgaatgcctagacccttatttttattaatttccaatagatgctgcctatgggctatattgctttagatgaacattagatatttaaagctcaagaggttcaaaatccaactcattatcttctctttctttcacctccctgctcctctccctatattactgattgcactgaacagcatggtccccaatgtagccatgcaaatgagaaacccagtggctccttgtggtacatgcatgcaagactgctgaagccagaaggatgactgattacgcctcatgggtggaggggaccactcctgggccttcgtgattgtcaggagcaagacctgagatgctccctgccttcagtgtcctctgcatctcccctttctaatgaagatccatagaatttgctacatttgagaattccaattaggaactcacatgttttatctgccctatcaattttttaaacttgctgaaaattaagttttttcaaaatctgtccttgtaaattactttttcttacagtgtcttggcatactatatcaactttgattctttgttacaacttttcttactcttttatcaccaaagtggcttttattctctttattattattattttcttttactactatattacgttgttattattttgttctctatagtatcaatttatttgatttagtttcaatttatttttattgctgacttttaaaataagtgattcggggggtgggagaacaggggagggagagcattaggacaaatacctaatgcatgtgggacttaaaacctagatgatgggttgataggtgcagcaaaccactatggcacacgtatacctgtgtaacaaacctacacattctgcacatgtatcccagaacgtaaagtaaaatttaaaaaaaagtga

SEQ ID NO: 10 - RNA, 21 nt, synthetic construct
  source 1..21: mol_type = other RNA
gcacctatatttactactttt

SEQ ID NO: 11 - RNA, 21 nt, synthetic construct
  source 1..21: mol_type = other RNA
aagtagtaaatataggtgctg

SEQ ID NO: 12 - RNA, 7733 nt, Homo sapiens
  source 1..7733: mol_type = mRNA
  CDS 123..6200: protein_id = 13; translation = MDNFAEGDFTVADYALLEDCPHVDDCVFAAEFMSNDYVRVTQLY CDGVGVQYKDYIQSERNLEFDICSIWCSKPISVLQDYCDAIKINIFWPLLFQHQNSSV ISRLHPCVDANNSRASEINLKKLQHLELMEDIVDLAKKVANDSFLIGGLLRIGCKIEN KILAMEEALNWIKYAGDVTILTKLGSIDNCWPMLSIFFTEYKYHITKIVMEDCNLLEE LKTQSCMDCIEEGELMKMKGNEEFSKERFDIAIIYYTRAIEYRPENYLLYGNRALCFL RTGQFRNALGDGKRATILKNTWPKGHYRYCDALSMLGEYDWALQANIKAQKLCKNDPE GIKDLIQQHVKLQKQIEDLQGRTANKDPIKAFYENRAYTPRSLSAPIFTTSLNFVEKE RDFRKINHEMANGGNQNLKVADEALKVDDCDCHPEFSPPSSQPPKHKGKQKSRNNESE KFSSSSPLTLPADLKNILEKQFSKSSRAAHQDFANIMKMLRSLIQDGYMALLEQRCRS AAQAFTELLNGLDPQKIKQLNLAMINYVLVVYGLAISLLGIGQPEELSEAENQFKRII EHYPSEGLDCLAYCGIGKVYLKKNRFLEALNHFEKARTLIYRLPGVLTWPTSNVIIEE SQPQKIKMLLEKFVEECKFPPVPDAICCYQKCHGYSKIQIYITDPDFKGFIRISCCQY CKIEFHMNCWKKLKTTTFNDKIDKDFLQGICLTPDCEGVISKIIIFSSGGEVKCEFEH KVIKEKVPPRPILKQKCSSLEKLRLKEDKKLKRKIQKKEAKKLAQERMEEDLRESNPP KNEEQKETVDNVQRCQFLDDRILQCIKQYADKIKSGIQNTAMLLKELLSWKVLSTEDY TTCFSSRNFLNEAVDYVIRHLIQENNRVKTRIFLHVLSELKEVEPKLAAWIQKLNSFG LDATGTFFSRYGASLKLLDFSIMTFLWNEKYGHKLDSIEGKQLDYFSEPASLKEARCL IWLLEEHRDKFPALHSALDEFFDIMDSRCTVLRKQDSGEAPFSSTKVKNKSKKKKPKD SKPMLVGSGTTSVTSNNEIITSSEDHSNRNSDSAGPFAVPDHLRQDVEEFEALYDQHS NEYVVRNKKLWDMNPKQKCSTLYDYFSQFLEEHGPLDMSNKMFSAEYEFFPEETRQIL EKAGGLKPFLLGCPRFVVIDNCIALKKVASRLKKKRKKKNIKTKVEEISKAGEYVRVK LQLNPAAREFKPDVKSKPVSDSSSAPAFENVKPKPVSANSPKPACEDVKAKPVSDNSS RQVSEDGQPKGVSSNSPKPGSEDANYKRVSCNSPKPVLEDVKPTYWAQSHLVTGYCTY LPFQRFDITQTPPAYINVLPGLPQYTSIYTPLASLSPEYQLPRSVPVVPSFVANDRAD KNAAAYFEGHHLNAENVAGHQIASETQILEGSLGISVKSHCSTGDAHTVLSESNRNDE HCGNSNNKCEVIPESTSAVTNIPHVQMVAIQVSWNIIHQEVNTEPYNPFEERQGEISR IEKEHQVLQDQLQEVYENYEQIKLKGLEETRDLEEKLKRHLEENKISKTELDWFLQDL EREIKKWQQEKKEIQERLKSLKKKIKKVSNASEMYTQKNDGKEKEHELHLDQSLEISN TLTNEKMKIEEYIKKGKEDYEESHQRAVAAEVSVLENWKESEVYKLQIMESQAEAFLK KLGLISRDPAAYPDMESDIRSWELFLSNVTKEIEKAKSQFEEQIKAIKNGSRLSELSK VQISELSFPACNTVHPELLPESSGDDGQGLVTSASDVTGNHAALHRDPSVFSAGDSPG EAPSALLPGPPPGQPEATQLTGPKRAGQAALSERSPVADRKQPVPPGRAARSSQSPKK PFNSIIEHLSVVFPCYNSTELAGFIKKVRSKNKNSLSGLSIDEIVQRVTEHILDEQKK KKPNPGKDKRTYEPSSATPVTRSSQGSPSVVVAPSPKTKGQKAEDVPVRIALGASSCE ICHEVFKSKNVRVLKCGHKYHKGCFKQWLKGQSACPACQGRDLLTEESPSGRGWPSQN QELPSCSSR
acccgctccctcccgccgtcggctgacgtggagggccggaggtggcggcggcggcggcggcggctgctgctgctgctgcccgcgtccgaggctcgcgggcggcgggcccggacttgtgcaccatggacaattttgctgagggagatttcactgtggcggattatgccttgttagaagattgccctcacgtggatgattgtgtctttgctgctgaatttatgagcaatgattatgttcgtgtgactcagctttactgtgatggggtgggtgtgcaatataaagattatatccaaagtgagaggaatttggaatttgacatctgcagtatatggtgtagtaaaccaatttctgtcctgcaagattattgcgatgccattaaaataaacatcttctggccacttctgtttcaacatcaaaacagttccgtaatatcacgattgcatccctgtgtggacgccaacaattcacgtgcttctgagataaatttgaagaaactacaacatcttgagttgatggaagatattgtggatttggcaaagaaagttgctaatgattcattccttattggaggcttattgagaattggttgtaaaatagaaaataaaatcttggcaatggaagaagctctgaattggataaaatatgcaggcgatgtaacaattctaactaaattaggatcaattgacaattgttggcctatgttaagtattttctttactgaatacaagtaccacataactaaaattgtaatggaagactgcaatttgcttgaagaacttaaaactcaaagttgtatggattgtatagaggaaggagaactaatgaaaatgaaaggaaatgaagagttttccaaagaaagatttgatatagctattatctattacaccagagccattgaatatagacctgaaaactaccttctttatggtaaccgagctctttgttttcttcgtactggacagtttagaaatgcactcggtgatggaaagagagccactattctgaagaacacttggccaaagggtcattatcgttattgtgatgctctttctatgctgggggaatatgactgggccctgcaagcaaacataaaagctcaaaaactctgtaaaaatgaccctgagggaatcaaggatctaattcagcagcatgtaaagttacaaaaacaaatagaagacctacaaggtcgaacagcaaataaggatccaattaaagccttttatgaaaacagggcctacacacctaggagtttatcagcacctatatttactacttcacttaactttgtggagaaggaaagagatttcagaaaaattaatcacgaaatggccaacggtggtaatcagaatctaaaggtggcggatgaggcgttgaaggtagatgattgtgactgtcatcctgaattttcaccaccatcaagtcagcctccaaaacataaaggaaaacaaaaatctcgaaacaatgaatcagaaaagttcagttctagttcaccattgactttaccagcagatttgaagaacatcttggagaaacagttttctaaatcttccagagctgcacaccaggattttgctaatataatgaaaatgctgagaagcttaattcaagatggctatatggccttattggagcagcgttgccgcagcgctgcacaggcctttacagagttgctgaacggtttagatcctcaaaaaataaagcaattgaacctggccatgattaactatgttttggtcgtctatggacttgccatttctctccttggaataggacagcctgaggaattatctgaagccgaaaaccagtttaagaggattattgaacactaccccagtgagggccttgattgcttggcctactgtggaattggaaaagtatatttgaaaaaaaacagatttctagaagctctcaatcactttgagaaagcaagaaccttgatttatcgtcttcctggagtgttaacttggcccacgagtaatgtgattattgaagagtctcagccacaaaaaataaagatgctgttagagaaatttgttgaagaatgcaagttccctccagtgccagatgccatttgttgctatcagaagtgccatggatattctaagatccagatatacataactgatccagactttaagggttttatacgcatcagctgttgccagtactgtaaaatagaatttcacatgaattgctggaagaagttaaaaactacaacctttaatgataaaattgacaaggattttctacaaggaatatgtcttacccctgactgtgaaggtgtcatttctaagattatcatcttcagcagtggtggtgaagttaaatgtgaatttgaacacaaggtcataaaagaaaaggttcctccaagacctattctgaaacagaaatgttctagcctagagaaactaagactgaaagaagacaaaaaattgaagagaaagatccaaaaaaaagaagcaaaaaagttagcacaagaaagaatggaggaggacttaagagaaagtaatccacccaaaaatgaagagcagaaagaaactgtagacaatgttcagcgttgtcagttccttgatgacagaattctacagtgtataaagcagtatgctgacaagattaaatccggcatacagaatacagccatgcttctcaaagaattgctttcttggaaagttttgagcacagaagactatacaacctgtttttctagcagaaattttctaaatgaagcagtggactatgttattcgccacttgattcaagaaaataacagagtaaagacaagaatatttctgcatgttttgagtgagcttaaagaagtggagcccaaattagccgcctggatccaaaaacttaatagctttggcttagatgccacaggaactttcttttctcgttatggagcatctcttaaactgcttgattttagtatcatgactttcctctggaatgagaaatatggtcacaaactagactctatagaaggaaagcaacttgattatttctctgagccagcatcattgaaggaagcccgttgtttaatatggctgctagaagaacacagagacaagttcccagcattgcatagtgctttagatgaattctttgatataatggacagccgctgtactgtgttaaggaaacaagatagtggtgaagcaccgtttagttcaaccaaggtgaaaaacaaaagcaagaaaaagaagccaaaggattcaaagcctatgttagttgggtctggaacaacttcagtaacttcaaataatgagatcatcacttcaagtgaagaccatagcaatcgaaattcagattctgcaggcccatttgcagtgcctgaccatcttcggcaagatgtagaagaattcgaagctctctatgaccaacacagtaacgaatatgttgtccgcaataagaagctatgggacatgaacccaaaacaaaaatgttcaactctatatgattacttctctcagtttttggaggaacatggtcccttggacatgagtaacaagatgttctctgcagaatatgagtttttcccagaagaaactcgacagatactagaaaaagcaggaggtttaaaaccttttctcttgggatgccctcgttttgttgtgattgacaactgtattgcactgaagaaggttgcatcacggctcaagaaaaaaaggaagaagaaaaacattaaaacaaaagtagaagaaatttcaaaagcaggggagtatgtacgagttaaactacaactgaatccagctgctagggaatttaaaccagatgtaaagtctaaaccagtgtcagattcatcttcagcaccagcttttgaaaatgtgaaacccaaacctgtgtctgcaaattctcccaagccagcttgtgaagatgtgaaggccaaaccagtatccgacaattcttctagacaagtttctgaggatgggcaacccaaaggggtctcttctaattctcctaaaccaggctctgaggatgcaaattacaagcgagtctcctgtaattcccccaaaccggttcttgaggatgtgaaaccaacttattgggctcaatcccatttggtcacaggatactgtacgtatcttcctttccagagatttgatatcacccagacaccgccagcatacataaacgtgttaccaggtttgccccagtacaccagcatatatacacccttggccagcctttctcctgaatatcagctaccaagatcagtaccagtggtgccgtcttttgtagccaatgacagagcagataaaaatgctgctgcctattttgagggtcatcatttgaatgctgagaatgttgctggtcaccagattgcctctgaaacacagatccttgagggctctttgggaatatctgtaaagtcacactgcagcacaggtgatgctcatacagtcctgagtgagtctaacagaaatgatgagcactgtggaaattctaacaacaaatgtgaagtaattccagaaagcaccagtgcagtaacaaacattccacacgtgcagatggttgccatacaggtatcttggaacataatacaccaagaagtcaatactgagccatataatccttttgaggaacgacaaggggaaatttcacggattgaaaaggagcaccaagtattacaagaccaacttcaagaagtgtatgaaaattatgagcagataaaacttaagggcttagaagagaccagggacctggaagagaagttgaaaaggcacttagaagaaaacaagatctcaaagacggaattagattggttccttcaagatttggaaagagaaattaaaaaatggcaacaggaaaaaaaagaaatccaagaaagactaaaatcactgaagaagaaaattaaaaaggtttcaaatgccagtgaaatgtatacccagaaaaatgatggaaaggaaaaggaacatgaattacatctggatcagtcccttgaaatcagcaacacacttacaaatgagaaaatgaaaatagaagagtatataaagaaagggaaagaggattatgaagagagtcatcagagagctgtggctgcagaggtatccgtacttgaaaactggaaggagagtgaagtgtataagctacagatcatggagtcacaagcagaagcctttctgaagaagctggggctgattagccgtgatcctgcagcatatcctgacatggagtctgatatacgttcatgggaattgtttctttctaatgttacaaaagaaattgagaaagcaaagtctcagtttgaagaacaaattaaggcaattaaaaatggttctcggctcagtgaactttctaaagtgcagatttctgagctttcatttcctgcctgtaacacggttcatcccgagttactccctgagtcttcaggcgacgatggccaagggcttgtgacttctgcaagcgacgtgactggaaaccacgcagcacttcacagggatcctagtgtgttctctgctggtgattccccaggggaggctccttctgcgctgttgccagggccaccccctggtcagcctgaagccactcagctgacagggccaaaacgggctggccaggcagctctgtcagaacgaagccctgtggctgatcggaagcagcctgttcctccaggacgtgctgcgcgttcaagccagtctccaaaaaagccgttcaatagtattattgagcacctgtcagtggtattcccatgttacaacagcactgagcttgctggttttattaaaaaagtgcgaagcaaaaacaagaactcactctcaggattgagtattgatgaaattgtccaaagagtgacagaacacattctagatgaacagaaaaagaaaaagccaaacccaggaaaggacaagaggacttatgagcccagctctgccacccccgtgaccaggtcctcccagggctcaccctcggtggttgttgcaccatcacccaaaaccaaggggcagaaagcagaagatgtccctgtgaggattgcactgggtgcaagttcctgtgaaatatgccacgaggtgttcaaatcaaaaaacgtgcgtgtgctcaaatgtgggcacaagtatcacaaagggtgctttaagcagtggcttaaagggcagagcgcttgcccggcctgccagggtcgtgatctcctgacagaagagtcaccttctggaagaggctggcccagtcagaatcaggagctgccttcctgctcttctaggtagtcacacttcactaaagtgtcatccaccagtgtgttgaatccgaagaatgacaattttctaccactggtgtaaaaaacaaacatttgaagacccttgtgcattgtgtgtcacaaagctaaatacatggaaatcgttaatatcgctgatattaagtaatttccccactctgagtgaatactttgatgattgccaacagtggctaataaaatgacggctaccacactcatgggtcactggggctgcgcagggctctttgaggtgggtggcttcttttggaaagtactatgaacgtctcgaagcagtattctagtgataagaattcttaacatagccaagcgccccacgtttgttccccacgtttgttccccttttctgtttgaaaaacctgttctggtagctccacaagagagatgatactgactttttaaattttttacaagagtctgtattcctgatatgcctatatttttcctcaaagattctgcattttaaggatgggcataagcaaactatattttaataatttatagttaatgttaaaatattggctgatttagaccaaaagattcaaatctcctctttgtgaaatcccatctgcatttgattttttattattttatgttcccccgttagattgttttaagtgtttgcttttcatcttttatagatgtaatctgattttcaaaaatcattaacactttttaattagtatcgactaagactttttccccctggaatcgaggctgtgtgtccgtcatcccagcccccggttggagcctgctctttgaactccgctgccttccttagcagcttctgtcctcttctgtgagtcagtcagcgagtgcttgggatccgcatccagccgtgctgagcacacaacaggctgtgtgtggaaatggccaccaccattctccttccccaccccaccacaaaaagagaagctgtgtctttagacaaccctgaggtatctgtgttacaatcgttctgtgtttgatatttgtgtaaagtatgcatgcagtcttgtactgtgacctaagaacaaaactgtaactgcattagaaaccatgaaaaaattagatattgttttgtgacttttagacagtggtaaatatagaaccatgaattctggtcacattccatttctctccaacatgaaggatcaaaaaatgtttttcaatgtgttctttgttccactggaaacttagagtcatgagtttatgagctgatttggtcaccttcctctgcctttgttcactgtgagttctgatgtcttagtgacttagttcttagaagctcacgccttagtttgaaacagattctccacggtggtccccaaaacactgtctgcatatccataagaattgagcgctatgggtgttaacgtgcatgaggatcagtttgcagcagcaagtacaaaaggagaagaggaacatccgttgaatgagtgtgttttgtacataacttcagatacttgtgaacatgccttatatttgtccaacaactgtcagaataaagaacattctaaaatgagaa

SEQ ID NO: 13 - protein, 2025 aa, Homo sapiens
MDNFAEGDFTVADYALLEDCPHVDDCVFAAEFMSNDYVRVTQLYCDGVGVQYKDYIQSERNLEFDICSIWCSKPISVLQDYCDAIKINIFWPLLFQHQNSSVISRLHPCVDANNSRASEINLKKLQHLELMEDIVDLAKKVANDSFLIGGLLRIGCKIENKILAMEEALNWIKYAGDVTILTKLGSIDNCWPMLSIFFTEYKYHITKIVMEDCNLLEELKTQSCMDCIEEGELMKMKGNEEFSKERFDIAIIYYTRAIEYRPENYLLYGNRALCFLRTGQFRNALGDGKRATILKNTWPKGHYRYCDALSMLGEYDWALQANIKAQKLCKNDPEGIKDLIQQHVKLQKQIEDLQGRTANKDPIKAFYENRAYTPRSLSAPIFTTSLNFVEKERDFRKINHEMANGGNQNLKVADEALKVDDCDCHPEFSPPSSQPPKHKGKQKSRNNESEKFSSSSPLTLPADLKNILEKQFSKSSRAAHQDFANIMKMLRSLIQDGYMALLEQRCRSAAQAFTELLNGLDPQKIKQLNLAMINYVLVVYGLAISLLGIGQPEELSEAENQFKRIIEHYPSEGLDCLAYCGIGKVYLKKNRFLEALNHFEKARTLIYRLPGVLTWPTSNVIIEESQPQKIKMLLEKFVEECKFPPVPDAICCYQKCHGYSKIQIYITDPDFKGFIRISCCQYCKIEFHMNCWKKLKTTTFNDKIDKDFLQGICLTPDCEGVISKIIIFSSGGEVKCEFEHKVIKEKVPPRPILKQKCSSLEKLRLKEDKKLKRKIQKKEAKKLAQERMEEDLRESNPPKNEEQKETVDNVQRCQFLDDRILQCIKQYADKIKSGIQNTAMLLKELLSWKVLSTEDYTTCFSSRNFLNEAVDYVIRHLIQENNRVKTRIFLHVLSELKEVEPKLAAWIQKLNSFGLDATGTFFSRYGASLKLLDFSIMTFLWNEKYGHKLDSIEGKQLDYFSEPASLKEARCLIWLLEEHRDKFPALHSALDEFFDIMDSRCTVLRKQDSGEAPFSSTKVKNKSKKKKPKDSKPMLVGSGTTSVTSNNEIITSSEDHSNRNSDSAGPFAVPDHLRQDVEEFEALYDQHSNEYVVRNKKLWDMNPKQKCSTLYDYFSQFLEEHGPLDMSNKMFSAEYEFFPEETRQILEKAGGLKPFLLGCPRFVVIDNCIALKKVASRLKKKRKKKNIKTKVEEISKAGEYVRVKLQLNPAAREFKPDVKSKPVSDSSSAPAFENVKPKPVSANSPKPACEDVKAKPVSDNSSRQVSEDGQPKGVSSNSPKPGSEDANYKRVSCNSPKPVLEDVKPTYWAQSHLVTGYCTYLPFQRFDITQTPPAYINVLPGLPQYTSIYTPLASLSPEYQLPRSVPVVPSFVANDRADKNAAAYFEGHHLNAENVAGHQIASETQILEGSLGISVKSHCSTGDAHTVLSESNRNDEHCGNSNNKCEVIPESTSAVTNIPHVQMVAIQVSWNIIHQEVNTEPYNPFEERQGEISRIEKEHQVLQDQLQEVYENYEQIKLKGLEETRDLEEKLKRHLEENKISKTELDWFLQDLEREIKKWQQEKKEIQERLKSLKKKIKKVSNASEMYTQKNDGKEKEHELHLDQSLEISNTLTNEKMKIEEYIKKGKEDYEESHQRAVAAEVSVLENWKESEVYKLQIMESQAEAFLKKLGLISRDPAAYPDMESDIRSWELFLSNVTKEIEKAKSQFEEQIKAIKNGSRLSELSKVQISELSFPACNTVHPELLPESSGDDGQGLVTSASDVTGNHAALHRDPSVFSAGDSPGEAPSALLPGPPPGQPEATQLTGPKRAGQAALSERSPVADRKQPVPPGRAARSSQSPKKPFNSIIEHLSVVFPCYNSTELAGFIKKVRSKNKNSLSGLSIDEIVQRVTEHILDEQKKKKPNPGKDKRTYEPSSATPVTRSSQGSPSVVVAPSPKTKGQKAEDVPVRIALGASSCEICHEVFKSKNVRVLKCGHKYHKGCFKQWLKGQSACPACQGRDLLTEESPSGRGWPSQNQELPSCSSR

SEQ ID NO: 14 - RNA, 21 nt, synthetic construct
  source 1..21: mol_type = other RNA
ggatattcttctccgcttatt

SEQ ID NO: 15 - RNA, 21 nt, synthetic construct
  source 1..21: mol_type = other RNA
taagcggagaagaatatccaa

SEQ ID NO: 16 - RNA, 5043 nt, Homo sapiens
  source 1..5043: mol_type = mRNA
  CDS 318..2330: protein_id = 17; translation = MAKRIKAKPTSDKPGSPYRSVTHFDSLAVIDIPGADTLDKLFDH AVSKFGKKDSLGTREILSEENEMQPNGKVFKKLILGNYKWMNYLEVNRRVNNFGSGLT ALGLKPKNTIAIFCETRAEWMIAAQTCFKYNFPLVTLYATLGKEAVVHGLNESEASYL ITSVELLESKLKTALLDISCVKHIIYVDNKAINKAEYPEGFEIHSMQSVEELGSNPEN LGIPPSRPTPSDMAIVMYTSGSTGRPKGVMMHHSNLIAGMTGQCERIPGLGPKDTYIG YLPLAHVLELTAEISCFTYGCRIGYSSPLTLSDQSSKIKKGSKGDCTVLKPTLMAAVP EIMDRIYKNVMSKVQEMNYIQKTLFKIGYDYKLEQIKKGYDAPLCNLLLFKKVKALLG GNVRMMLSGGAPLSPQTHRFMNVCFCCPIGQGYGLTESCGAGTVTEVTDYTTGRVGAP LICCEIKLKDWQEGGYTINDKPNPRGEIVIGGQNISMGYFKNEEKTAEDYSVDENGQR WFCTGDIGEFHPDGCLQIIDRKKDLVKLQAGEYVSLGKVEAALKNCPLIDNICAFAKS DQSYVISFVVPNQKRLTLLAQQKGVEGTWVDICNNPAMEAEILKEIREAANAMKLERF EIPIKVRLSPEPWTPETGLVTDAFKLKRKELRNHYLKDIERMYGGK
cgggattcggctggctctgccacaccaccgcgcgcccccgctccgcccgcccctccgggcgcgtcttttccgggctcgcgctgagtcccgcctccgccggctgtccgggtgcgcgcgcgccgctgcggctttttctctggcctccgccgcgcgctcctcctcgtcccagcgctagcgggcacgcggttcctttttgcgagctttccgagtgccaggcgccggccggctgcgaagacgcggtgggccgcccctccgattgaaatcacagaagatattcgtgttcttcttaagagaaaaagaggacatttaaaaacgctatggcaaagagaataaaagctaagcccacttcagacaaacctggaagtccatatcgctctgtcacacacttcgactcactagctgtaatagacatccctggagcagatactctggataaattatttgaccatgctgtatccaagtttgggaagaaggacagccttgggaccagggaaatcctaagtgaagaaaatgaaatgcagccaaatggaaaagtttttaagaagttaattcttgggaattataaatggatgaactatcttgaagtgaatcgcagagtgaataactttggtagtggactcactgcactgggactaaaaccaaagaacaccattgccatcttctgtgagaccagggccgaatggatgattgcagcacagacctgctttaagtacaactttcctcttgtgactttatatgccacacttggcaaagaagcagtagttcatgggctaaatgaatctgaggcttcctatctgattaccagtgttgaacttctggaaagtaaacttaagactgcattgttagatatcagttgtgttaaacatatcatttatgtggacaataaggctatcaataaagcagagtaccctgaaggatttgagattcacagcatgcaatcagtagaagagttgggatctaacccagaaaacttgggcattcctccaagtagaccaacgccttcagacatggccattgttatgtatactagtggttctactggccgacctaagggagtgatgatgcatcatagcaatttgatagctggaatgacaggccagtgtgaaagaatacctggactgggaccgaaggacacatatattggctacttgcctttggctcatgtgctagaactgacagcagagatatcttgctttacctatggctgcaggattggatattcttctccgcttacactctctgaccagtccagcaaaattaaaaaaggaagcaaaggagactgtactgtactgaagcccacacttatggctgctgttccggaaatcatggatagaatttataagaatgttatgagcaaagtccaagagatgaattatattcagaaaactctgttcaagatagggtatgattacaaattggaacagatcaaaaagggatatgatgcacctctttgcaatctgttactgtttaaaaaggtcaaggccctgctgggagggaatgtccgcatgatgctgtctggaggggccccgctatctcctcagacacaccgattcatgaatgtctgcttctgctgcccaattggccagggttatggactgacagaatcatgtggtgctgggacagttactgaagtaactgactatactactggcagagttggagcacctcttatttgctgtgaaattaagctaaaagactggcaagaaggcggttatacaattaatgacaagccaaaccccagaggtgaaatcgtaattggtggacagaacatctccatgggatattttaaaaatgaagagaaaacagcagaagattattctgtggatgaaaatggacaaaggtggttttgcactggtgatattggagaattccatcccgatggatgtttacagattatagatcgtaagaaagatctagtgaagttacaagcaggagagtatgtatctcttgggaaagtagaagctgcactgaagaattgtccacttattgacaacatctgtgcttttgccaaaagtgatcagtcctatgtgatcagttttgtggttcctaaccagaaaaggttgacacttttggcacaacagaaaggggtagaaggaacttgggttgatatctgcaataatcctgctatggaagctgaaatactgaaagaaattcgagaagctgcaaatgccatgaaattggagcgatttgaaattccaatcaaggttcgattaagcccagagccatggacccctgaaactggtttggtaactgatgctttcaaactgaaaaggaaggagctgaggaaccattacctcaaagacattgaacgaatgtatgggggcaaataaaatgttgttgtcttattgacagttgtgcaggaggtagcctggtggttttcaacctctagaattttaagcctttgttgaactgttagaatgtaaggtatatcattctaaagatagagtaaaaagaaaacaaaaccaaaagttattaaaattgttgtccggtttactttaacttagttttgcatagttctagtgcagctgaaattgaaaagttatttccctttagctgtgttattatagagcagaaattctgtttttaaaaattagcctaagatatacttgtttttgtaaagaaaaatatttaatgttgaacaaaataaattggagttggagtagaatgtagtttgaggaaatttgcagcttccaatgcctcttgtcttcctatttcagaagtttaaatattaagcatgacagaaaatatgtattaacactactcaaagcaaaagtgctgcagggctttaaaattctcttccaaccatttatcttgaaggaaaaattcaatagtaatataatacacaaaatcaaataataccttagaaggtattaagattataattgttgcataggttagatatagagtcattgtaatgttgtgaataattacagtgcctaaaataagaatagaacaacatatacaacaccaaaaaatatctagtaatatatttaaagggaaattgagctgctttttttgaaactttgagatctaaaaataactgtaattatttgaatgactaagaggaaagtacattttttgaaatgctgaaaattgcctttctgtgtttattcaaactgaaaagctgagaccaagagcaaggaaggtaaaaagttaacaggcaaacattttctcttagaaaaggtgataaaatcataagtatttggaattagaacccttgcacagcactgaacctgggaaagagatttaaactctgaatttatctttgataacagggattgattttaaaatgtacatgtattaaattacatttgtaatttaaggtctgtttgctgttgctgattttattcttgatcagtagtttgcatttcagaaagcctttcattttgctttaagtttagcaaagcggggttataatgaatgacttccccaatatcttgcttgaacttacagtgattaacttggatgagttttgggaagttaaagggaagaaaacactgttatcattttttcctgtttgggaagagcttagaaactggaaatactagatttgggagaagggcagagttacttgataagggacttgatgtttgtgcagtaacttgggagtgtggtttctttttgaatctttaattaaaacctgggattatatatccctgataaatattcacacttgaaccatagttactgtaaaatgcaaaaaatcttaatactgttattctttgcactttttcttaatcattttttatatatatgcatatatatatgtgtgtgtgtgtgttgcttatgttgttttgtacagatgtgggccaccattgcaacaaaatacattctttttgctctaaaatatttatgaagaaaatacttaaatgttatgtatatggtggtaataagggaaaaatcaagtattataaacaagaatgaaggtttttgtaaagatttctgttcagcgttttgcaaggtaaaattttaggcaagttttccctgaagttatgtgtatgtgagtattctcattcttcccaacttgcctttgaagagtgaaataccattattatcaagtagactactgttcagcttttattccttccctggttgtttatcccttaggaatgagtttcttagactttcccaatatgtgattttttttcccatttagaatggtgattttaaatgtgtgagtgcatgtactatcttatctcagatatttgcacccccaatctgcccccaactcccaaaagctagaacactgccaactgatctgttataggtcctttagaaacacataattaacacttaaggttgggtgctgctaattctttgcaaaaatccaaatattgttaagggaccagggagatgccactaccccttgattttccatctaaaaatatacatgtttatgtaaacaaatctttccatatccatagtgacttttcaagtatttaagcctaaagattttgatctcacatttttatacctgtttaaattgctcacagttattacatacacatcagccatcaactaaagttgtactttaaaaatttactacaatatgtacatttctaagtcaaacacttgtgacttttgctttaattccatgaatgttcctgcctccttgatatttgtatttattctttttttctctagagtagaggtataattgtgtgatatttcagaaatacagataaatgattcaaaaagtcacagttaaggagaatcatgtttctttgatcatgaataactgattagtaagtcttgcctatattttcctgatagcatatgacaaatgtttctaaggtaacaagatgagaacagataaagattgtgtggtgttttggatttggagagaaatattttaatttttaaatgcagttacaaattataatgtattcatatttgtactttctgttaaaatgcatgattgcagaattgtttagattttgtgtttattcttgatgaaaagctttgtttgttcttgtttttaagtttgcactcaaatcttaagaaataaatccacccatgttatcaaaaaaaaaaaaaaaaaaa

SEQ ID NO: 17 - protein, 670 aa, Homo sapiens
MAKRIKAKPTSDKPGSPYRSVTHFDSLAVIDIPGADTLDKLFDHAVSKFGKKDSLGTREILSEENEMQPNGKVFKKLILGNYKWMNYLEVNRRVNNFGSGLTALGLKPKNTIAIFCETRAEWMIAAQTCFKYNFPLVTLYATLGKEAVVHGLNESEASYLITSVELLESKLKTALLDISCVKHIIYVDNKAINKAEYPEGFEIHSMQSVEELGSNPENLGIPPSRPTPSDMAIVMYTSGSTGRPKGVMMHHSNLIAGMTGQCERIPGLGPKDTYIGYLPLAHVLELTAEISCFTYGCRIGYSSPLTLSDQSSKIKKGSKGDCTVLKPTLMAAVPEIMDRIYKNVMSKVQEMNYIQKTLFKIGYDYKLEQIKKGYDAPLCNLLLFKKVKALLGGNVRMMLSGGAPLSPQTHRFMNVCFCCPIGQGYGLTESCGAGTVTEVTDYTTGRVGAPLICCEIKLKDWQEGGYTINDKPNPRGEIVIGGQNISMGYFKNEEKTAEDYSVDENGQRWFCTGDIGEFHPDGCLQIIDRKKDLVKLQAGEYVSLGKVEAALKNCPLIDNICAFAKSDQSYVISFVVPNQKRLTLLAQQKGVEGTWVDICNNPAMEAEILKEIREAANAMKLERFEIPIKVRLSPEPWTPETGLVTDAFKLKRKELRNHYLKDIERMYGGK

SEQ ID NO: 18 - RNA, 21 nt, synthetic construct
  source 1..21: mol_type = other RNA
aattcgatatgttcagcaatt

SEQ ID NO: 19 - RNA, 21 nt, synthetic construct
  source 1..21: mol_type = other RNA
ttgctgaacatatcgaatttg

SEQ ID NO: 20 - RNA, 19 nt, synthetic construct
  source 1..19: mol_type = other RNA
ggtgcttcgtctgaatcat

SEQ ID NO: 21 - RNA, 19 nt, synthetic construct
  source 1..19: mol_type = other RNA
atgattcagacgaagcacc

SEQ ID NO: 22 - RNA, 19 nt, synthetic construct
  source 1..19: mol_type = other RNA
gcgcagatcaaagcctttg

SEQ ID NO: 23 - RNA, 19 nt, synthetic construct
  source 1..19: mol_type = other RNA
caaaggctttgatctgcgc

SEQ ID NO: 24 - RNA, 888 nt, Homo sapiens
  source 1..888: mol_type = mRNA
ccagccccgccgcgatgagcctcggccgcctttgccgcctactgaagccggcgctgctctgtggggctctggccgcgcctggcctggccgggaccatgtgcgcgtcccgggacgactggcgctgtgcgcgctccatgcacgagttttccgccaaggacatcgacgggcacatggttaacctggacaagtaccggggcttcgtgtgcatcgtcaccaacgtggcctcccagtgaggcaagaccgaagtaaactacactcagctcgtcgacctgcacgtccgatacgctgagtgtggtttgcggatcctggccttcccgtgtaaccagttcgggaagcaggagccagggagtaacgaagagatcaaagagttcgccgcgggctacaacgtcaaattcgatatgttcagcaagatctgcgtgaacggggacgacgcccacccgctgtggaagtggatgaagatccaacccaagggcaagggcatcctgggaaatgccatcaagtggaacttcaccaagttcctcatcgacaagaacggctgcgtggtgaagcgctacggacccatggaggagcccctggtgatagagaaggacctgccccactatttctagctccacaagtgtgtggccccgcccgagcccctgcccacgcccttggagccttccaccggcactcatgacggcctgcctgcaaacctgctggtggggcagacccgaaaatccagcgtgcaccccgccggaggaaggtcccatggcctgctgggcttggctcggcgcccccacccctggctaccttgtgggaataaacagacaaattagcaaaaaaaaaaaaaaaaaaaaaaaaaaaaaaaaaaaaaaaaaaaaaaaaaaaaaaaaaaaaaaaaaaaaaaaa

SEQ ID NO: 25 - protein, 197 aa, Homo sapiens
MSLGRLCRLLKPALLCGALAAPGLAGTMCASRDDWRCARSMHEFSAKDIDGHMVNLDKYRGFVCIVTNVASQXGKTEVNYTQLVDLHVRYAECGLRILAFPCNQFGKQEPGSNEEIKEFAAGYNVKFDMFSKICVNGDDAHPLWKWMKIQPKGKGILGNAIKWNFTKFLIDKNGCVVKRYGPMEEPLVIEKDLPHYF

SEQ ID NO: 26 - RNA, 21 nt, synthetic construct
  source 1..21: mol_type = other RNA
cccttaaactcgaacccaatt

SEQ ID NO: 27 - RNA, 21 nt, synthetic construct
  source 1..21: mol_type = other RNA
ttgggttcgagtttaagggac

SEQ ID NO: 28 - RNA, 21 nt, synthetic construct
  source 1..21: mol_type = other RNA
tgaatttcttgggttcgagtt

SEQ ID NO: 29 - RNA, 21 nt, synthetic construct
  source 1..21: mol_type = other RNA
aaaccttttctccaggccttt

SEQ ID NO: 30 - RNA, 21 nt, synthetic construct
  source 1..21: mol_type = other RNA
tgaatttcttgggttcgagtt

SEQ ID NO: 31 - RNA, 2237 nt, Homo sapiens
  source 1..2237: mol_type = mRNA
attacttttccccccccctaactaacggactattattgttgttgttttaaatttagctcttagggcttagctatttgggttttcttgcggtgtccggctcccgtctccctggctcccccgcccgccctgcggccccagcgcccctcgctctcatccagcccgcgaggagtgcgggcgccgcgccgcctttaaagcgaggccagggagcgaggcggtgaccggccgagatccggccctcgcctcctccctcggtggcgctagggctcccggcctctcttcctcagtgcgggcggagaagcgaaagcggatcgtcctcggctgccgccgccttctccgggactcgcgcgcccctccccgcgcgcccacccacccagtccggctggactgcggcagccgcgcggctcaccccggcaggatgttcgcagccgggctggctcccttctacgcctccaacttcagcctctggtcggccgcttactgctcctcggccggcccaggcggctgctccttccccttggaccccgccgccgtcaaaaagccctccttctgcatcgcagacattctgcacgccggcgtgggggatctgggggcggccccggagggcctggcaggggcctcggccgccgccctcaccgcgcacttgggctcggttcacccgcacgcctctttccaagcggcggccagatccccgcttcgacccaccccagtggtggcgccctccgaagtcccggctggcttcccgcagcggctgtctccgctctcagccgcctaccaccaccatcacccgcaacaacaacagcagcagcaacagccgcagcagcaacagcctccgcctccgccccgggctggcgccctgcagcccccggcctcggggacgcgagtggttccgaacccccaccacagtggctctgccccggccccctccagcaaagacctcaaatttggaattgaccgcattttatctgcagaatttgacccaaaagtcaaagaaggcaacacgctgagagatctcacttccctgctaaccggtgggcggcccgccggggtgcacctctcaggcctgcagccctcggccggccagttcttcgcatctctagatcccattaacgaggcttctgcaatcctgagtcccttaaactcgaacccaagaaattcagttcagcatcagttccaagacacgtttccaggtccctatgctgtgctcacgaaggacaccatgccgcagacgtacaaaaggaagcgttcatggtcgcgcgctgtgttctccaacctgcagaggaaaggcctggagaaaaggtttgagattcagaagtacgtgaccaagccggaccgaaagcagctggcggcgatgctgggcctcacggacgcacaggtgaaggtgtggttccagaaccggcggatgaagtggcggcactccaaggaggcccaggcccaaaaggacaaggacaaggaggctggcgagaagccatcaggtggagccccggctgcggatggcgagcaggacgagaggagccccagccgttctgaaggcgaggctgagagcgagagcagcgactccgagtccctggacatggcccccagcgacacggagcggactgaggggagtgagcgttctctgcaccaaacaacagttattaaggccccggtcactggcgccctcattaccgccagcagtgctgggagtggtgggagcagcggcggcggcggcaatagtttcagcttcagcagcgccagcagtcttagtagcagcagcaccagtgcgggttgcgccagcagccttggcggcggcggcgcctcggagcttctccctgcaacacagcccacagccagcagcgctcccaaaagccccgagccagcccaaggcgcgcttggctgcttatagactgtactagggcggaggggatccgggccttgcgtgcagcctcccaaccatgggctgggttttgtgcttactgtatgttggcgacttggtagggcaggagacgcagcgtggagcctacctcccgacattcacgcttcgccccacgctgctccgactggctgcagcggacactgcccaaagcagaggggagtctcagtgtcctgctagccagccgaacacttctctccggaagcaggctggttcgactgtgaggtgtttgactaaactgtttctctgactcgccccagaggtcgtggctcaaaggcacttaggacgccttaaatttgtaaataaaatgtttactacggtttgtaaa

SEQ ID NO: 32 - protein, 488 aa, Homo sapiens
MFAAGLAPFYASNFSLWSAAYCSSAGPGGCSFPLDPAAVKKPSFCIADILHAGVGDLGAAPEGLAGASAAALTAHLGSVHPHASFQAAARSPLRPTPVVAPSEVPAGFPQRLSPLSAAYHHHHPQQQQQQQQPQQQQPPPPPRAGALQPPASGTRVVPNPHHSGSAPAPSSKDLKFGIDRILSAEFDPKVKEGNTLRDLTSLLTGGRPAGVHLSGLQPSAGQFFASLDPINEASAILSPLNSNPRNSVQHQFQDTFPGPYAVLTKDTMPQTYKRKRSWSRAVFSNLQRKGLEKRFEIQKYVTKPDRKQLAAMLGLTDAQVKVWFQNRRMKWRHSKEAQAQKDKDKEAGEKPSGGAPAADGEQDERSPSRSEGEAESESSDSESLDMAPSDTERTEGSERSLHQTTVIKAPVTGALITASSAGSGGSSGGGGNSFSFSSASSLSSSSTSAGCASSLGGGGASELLPATQPTASSAPKSPEPAQGALGCL

SEQ ID NO: 33 - RNA, 19 nt, synthetic construct
  source 1..19: mol_type = other RNA
gcttgtacctgcaggatct

SEQ ID NO: 34 - RNA, 19 nt, synthetic construct
  source 1..19: mol_type = other RNA
cgtccaagcagaggagcaa

SEQ ID NO: 35 - RNA, 21 nt, synthetic construct
  source 1..21: mol_type = other RNA
gccacagcatacatcctgttt

SEQ ID NO: 36 - RNA, 21 nt, synthetic construct
  source 1..21: mol_type = other RNA
acaggatgtatgctgtggctt

SEQ ID NO: 37 - RNA, 3720 nt, Homo sapiens
  source 1..3720: mol_type = mRNA
aactcgctgtagtaattccagcgagaggcagagggagcgagcgggcggccggctagggtggaagagccgggcgagcagagctgcgctgcgggcgtcctgggaagggagatccggagcgaatagggggcttcgcctctggcccagccctcccgctgatcccccagccagcggtccgcaacccttgccgcatccacgaaactttgcccatagcagcgggcgggcactttgcactggaacttacaacacccgagcaaggacgcgactctcccgacgcggggaggctattctgcccatttggggacacttccccgccgctgccaggacccgcttctctgaaaggctctccttgcagctgcttagacgctggatttttttcgggtagtggaaaaccagcagcctcccgcgacgatgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcgtaaggaaaagtaaggaaaacgattccttctaacagaaatgtcctgagcaatcacctatgaacttgtttcaaatgcatgatcaaatgcaacctcacaaccttggctgagtcttgagactgaaagatttagccataatgtaaactgcctcaaattggactttgggcataaaagaacttttttatgcttaccatcttttttttttctttaacagatttgtatttaagaattgtttttaaaaaattttaagatttacacaatgtttctctgtaaatattgccattaaatgtaaataactttaataaaacgtttatagcagttacacagaatttcaatcctagtatatagtacctagtattataggtactataaaccctaattttttttatttaagtacattttgctttttaaagttgatttttttctattgtttttagaaaaaataaaataactggcaaatatatcattgagccaaatcttaagttgtgaatgttttgtttcgtttcttccccctcccaaccaccaccatccctgtttgttttcatcaattgccccttcagagggtggtcttaagaaaggcaagagttttcctctgttgaaatgggtctgggggccttaaggtctttaagttcttggaggttctaagatgcttcctggagactatgataacagccagagttgacagttagaaggaatggcagaaggcaggtgagaaggtgagaggtaggcaaaggagatacaagaggtcaaaggtagcagttaagtacacaaagaggcataaggactggggagttgggaggaaggtgaggaagaaactcctgttactttagttaaccagtgccagtcccctgctcactccaaacccaggaattctgcccagttgatggggacacggtgggaaccagcttctgctgccttcacaaccaggcgccagtcctgtccatgggttatctcgcaaaccccagaggatctctgggaggaatgctactattaaccctatttcacaaacaaggaaatagaagagctcaaagaggttatgtaacttatctgtagccacgcagataatacaaagcagcaatctggacccattctgttcaaaacacttaacccttcgctatcatgccttggttcatctgggtctaatgtgctgagatcaagaaggtttaggacctaatggacagactcaagtcataacaatgctaagctctatttgtgtcccaagcactcctaagcattttatccctaactctacatcaaccccatgaaggagatactgttgatttccccatattagaagtagagagggaagctgaggcacacaaagactcatccacatgcccaagattcactgatagggaaaagtggaagcgagatttgaacccaggctgtttactcctaacctgtccaagccacctctcagacgacggtaggaatcagctggctgcttgtgagtacaggagttacagtccagtgggttatgttttttaagtctcaacatctaagcctggtcaggcatcagttcccctttttttgtgatttattttgtttttattttgttgttcattgtttaatttttccttttacaatgagaaggtcaccatcttgactcctaccttagccatttgttgaatcagactcatgacggctcctgggaagaagccagttcagatcataaaataaaacatatttattctttgtcatgggagtcattattttagaaactacaaactctccttgcttccatccttttttacatactcatgacacatgctcatcctgagtccttgaaaaggtatttttgaacatgtgtattaattataagcctctgaaaacctatggcccaaaccagaaatgatgttgattatataggtaaatgaaggatgctattgctgttctaattacctcattgtctcagtctcaaagtaggtcttcagctccctgtactttgggattttaatctaccaccacccataaatcaataaataattactttctttg

SEQ ID NO: 38 - protein, 454 aa, Homo sapiens
MDFFRVVENQQPPATMPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCA

SEQ ID NO: 39 - RNA, 21 nt, synthetic construct
  source 1..21: mol_type = other RNA
cggtgaatgaacttcagaatt

SEQ ID NO: 40 - RNA, 21 nt, synthetic construct
  source 1..21: mol_type = other RNA
gctgctgagaagtcgattatt

SEQ ID NO: 41 - RNA, 19 nt, synthetic construct
  source 1..19: mol_type = other RNA
gcaaaggattcggaaactt

SEQ ID NO: 42 - RNA, 19 nt, synthetic construct
  source 1..19: mol_type = other RNA
ggtgaaacttgaagctcat

SEQ ID NO: 43 - RNA, 19 nt, synthetic construct
  source 1..19: mol_type = other RNA
ccagacacctgatgagaat

SEQ ID NO: 44 - RNA, 21 nt, synthetic construct
  source 1..21: mol_type = other RNA
gcaggaattgacgctgtataa

SEQ ID NO: 45 - RNA, 21 nt, synthetic construct
  source 1..21: mol_type = other RNA
cggtgaatgaacttcagaatt

SEQ ID NO: 46 - RNA, 21 nt, synthetic construct
  source 1..21: mol_type = other RNA
tgttgtagtctcacagtataa

SEQ ID NO: 47 - RNA, 4274 nt, Homo sapiens
  source 1..4274: mol_type = mRNA
agtgtgatattttcacagtccgctggccgaagccaaaggggttgggaaggaagagacaaaaagtagttttttcccctcctcctcctcctttctcttgctaatcccgcctcctcctaagacttaggaagactggtggatgcgtttgggttgtagctaggctttttcttttctttctcttttaaaacacatctagacaaggaaaaaacaagcctcggatctgatttttcactcctcgttcttgtgcttggttcttactgtgtttgtgtattttaaaggcgagaagacgaggggaacaaaaccagctggatccatccatcaccgtgggtggttttaatttttcgttttttctcgttatttttttttaaacaaccactcttcacaatgaacaaactgtatatcggaaacctcagcgagaacgccgccccctcggacctagaaagtatcttcaaggacgccaagatcccggtgtcgggacccttcctggtgaagactggctacgcgttcgtggactgcccggacgagagctgggccctcaaggccatcgaggcgctttcaggtaaaatagaactgcacgggaaacccatagaagttgagcactcggtcccaaaaaggcaaaggattcggaaacttcagatacgaaatatcccgcctcatttacagtgggaggtgctggatagtttactagtccagtatggagtggtggagagctgtgagcaagtgaacactgactcggaaactgcagttgtaaatgtaacctattccagtaaggaccaagctagacaagcactagacaaactgaatggatttcagttagagaatttcaccttgaaagtagcctatatccctgatgaaatggccgcccagcaaaaccccttgcagcagccccgaggtcgccgggggcttgggcagaggggctcctcaaggcaggggtctccaggatccgtatccaagcagaaaccatgtgatttgcctctgcgcctgctggttcccacccaatttgttggagccatcataggaaaagaaggtgccaccattcggaacatcaccaaacagacccagtctaaaatcgatgtccaccgtaaagaaaatgcgggggctgctgagaagtcgattactatcctctctactcctgaaggcacctctgcggcttgtaagtctattctggagattatgcataaggaagctcaagatataaaattcacagaagagatccccttgaagattttagctcataataactttgttggacgtcttattggtaaagaaggaagaaatcttaaaaaaattgagcaagacacagacactaaaatcacgatatctccattgcaggaattgacgctgtataatccagaacgcactattacagttaaaggcaatgttgagacatgtgccaaagctgaggaggagatcatgaagaaaatcagggagtcttatgaaaatgatattgcttctatgaatcttcaagcacatttaattcctggattaaatctgaacgccttgggtctgttcccacccacttcagggatgccacctcccacctcagggcccccttcagccatgactcctccctacccgcagtttgagcaatcagaaacggagactgttcatctgtttatcccagctctatcagtcggtgccatcatcggcaagcagggccagcacatcaagcagctttctcgctttgctggagcttcaattaagattgctccagcggaagcaccagatgctaaagtgaggatggtgattatcactggaccaccagaggctcagttcaaggctcagggaagaatttatggaaaaattaaagaagaaaactttgttagtcctaaagaagaggtgaaacttgaagctcatatcagagtgccatcctttgctgctggcagagttattggaaaaggaggcaaaacggtgaatgaacttcagaatttgtcaagtgcagaagttgttgtccctcgtgaccagacacctgatgagaatgaccaagtggttgtcaaaataactggtcacttctatgcttgccaggttgcccagagaaaaattcaggaaattctgactcaggtaaagcagcaccaacaacagaaggctctgcaaagtggaccacctcagtcaagacggaagtaaaggctcaggaaacagcccaccacagaggcagatgccaaaccaaagacagattgcttaaccaacagatgggcgctgaccccctatccagaatcacatgcacaagtttttacctagccagttgtttctgaggaccaggcaacttttgaactcctgtctctgtgagaatgtatactttatgctctctgaaatgtatgacacccagctttaaaacaaacaaacaaacaaacaaaaaaagggtgggggagggagggaaagagaagagctctgcacttccctttgttgtagtctcacagtataacagatattctaattcttcttaatattcccccataatgccagaaattggcttaatgatgctttcactaaattcatcaaatagattgctcctaaatccaattgttaaaattggatcagaataattatcacaggaacttaaatgttaagccattagcatagaaaaactgttctcagttttatttttacctaacactaacatgagtaacctaagggaagtgctgaatggtgttggcaggggtattaaacgtgcatttttactcaactacctcaggtattcagtaatacaatgaaaagcaaaattgttccttttttttgaaaattttatatactttataatgatagaagtccaaccgttttttaaaaaataaatttaaaatttaacagcaatcagctaacaggcaaattaagatttttacttctggctggtgacagtaaagctggaaaattaatttcagggttttttgaggcttttgacacagttattagttaaatcaaatgttcaaaaatacggagcagtgcctagtatctggagagcagcactaccatttattctttcatttatagttgggaaagtttttgacggtactaacaaagtggtcgcaggagattttggaacggctggtttaaatggcttcaggagacttcagttttttgtttagctacatgattgaatgcataataaatgctttgtgcttctgactatcaatacctaaagaaagtgcatcagtgaagagatgcaagactttcaactgactggcaaaaagcaagctttagcttgtcttataggatgcttagtttgccactacacttcagaccaatgggacagtcatagatggtgtgacagtgtttaaacgcaacaaaaggctacatttccatggggccagcactgtcatgagcctcactaagctattttgaagatttttaagcactgataaattaaaaaaaaaaaattagactccaccttaagtagtaaagtataacaggatttctgtatactgtgcaatcagttctttgaaaaaaaagtcaaaagatagagaatacaagaaaagtttttgggatataatttgaatgactgtgaaaacatatgacctttgataacgaactcatttgctcactccttgacagcaaagcccagtacgtacaattgtgttgggtgtgggtggtctccaaggccacgctgctctctgaattgattttttgagttttgtttgtaagatgatcacagtcatgttacactgatctaaaggacatatatataaccctttaaaaaaaaaatcactgcctcattcttatttcaagatgaatttctatacagactagatgtttttctgaagatcaattagacattttgaaaatgatttaaagtgttttccttaatgttctctgaaaacaagtttcttttgtagttttaaccaaaaaagtgccctttttgtcactggattctcctagcattcatgatttttttttcatacaatgaattaaaattgctaaaatcatggactggctttctggttggatttcaggtaagatgtgtttaaggccagagcttttctcagtatttgatttttttccccaatatttgattttttaaaaatatacacataggtgctgcatttatatctgctggtttaaattctgtcatatttcacttctagccttttagtatggcaaatcatattttacttttacttaagcatttgtaatttggagtatctggtactagctaagaaataattctataattgagttttgtactcaccatatatggatcattcctcatgtataatgtgccccaaatgcagcttcattttccagataccttgacgcagaataaattttttcatcatttaggtgca

SEQ ID NO: 48 - protein, 579 aa, Homo sapiens
MNKLYIGNLSENAAPSDLESIFKDAKIPVSGPFLVKTGYAFVDCPDESWALKAIEALSGKIELHGKPIEVEHSVPKRQRIRKLQIRNIPPHLQWEVLDSLLVQYGVVESCEQVNTDSETAVVNVTYSSKDQARQALDKLNGFQLENFTLKVAYIPDEMAAQQNPLQQPRGRRGLGQRGSSRQGSPGSVSKQKPCDLPLRLLVPTQFVGAIIGKEGATIRNITKQTQSKIDVHRKENAGAAEKSITILSTPEGTSAACKSILEIMHKEAQDIKFTEEIPLKILAHNNFVGRLIGKEGRNLKKIEQDTDTKITISPLQELTLYNPERTITVKGNVETCAKAEEEIMKKIRESYENDIASMNLQAHLIPGLNLNALGLFPPTSGMPPPTSGPPSAMTPPYPQFEQSETETVHLFIPALSVGAIIGKQGQHIKQLSRFAGASIKIAPAEAPDAKVRMVIITGPPEAQFKAQGRIYGKIKEENFVSPKEEVKLEAHIRVPSFAAGRVIGKGGKTVNELQNLSSAEVVVPRDQTPDENDQVVVKITGHFYACQVAQRKIQEILTQVKQHQQQKALQSGPPQSRRK